SEQUENCE LISTING (95 sequences)

SEQ ID NO: 1 - RNA, 2692 nt, unidentified
  source 1..2692: mol_type = viral cRNA; note = Citrus yellow vein-associated virus [CYVaV]
gggtaaatatggatccttcatctttgccccgtgcctgttggcatcatgccagacaggtgtttcgagcatcaactagcttctcaagagaggtggttcgcgctgctcgtagatgggttaccatgcccaccagtcgccatgcatatgacttttcaacgagtctaggcattgtgattgctgagcctgcagctcgtttacgacgccgtctgccctctgtacgaaagtgcgcagagaagttagtagtccacaagcaagtcgacactttggtggacgaatggtgctctggaattcccaaccctgatatcgtagaagttggttgggcactccgtctgagggaccgtttcggtcttcctcccgcttctgagcctacccggctcagtggtgagagatgggtgctcaaacaactcaatggggtagatcctgagtcatggaatgctgatcttggtaggtcagttcatatccaaggagactacgccccagggaggaatgcccatatcgctcaggtcgcggcgaccttgtggttaactaggaccttgcatgacaaggccttggctcgccaccagggttttcgcgatttgcagtgattggggtcgacgggctagaggcaaaagcagtgcctctagcttctggactccgactgcttccggttccgcgacccggacaaagtcgacgactgtctcagaccttgttacttccaacacctcgtgctcaattcgtgaatcacgcgtgctcggctaacaaccttggacgtgtgatgaccacacgtgtgttgcagtacaagggccgagatccgatccttccctcttctgaagcccttcaccgacttaaccttcggatagctgagctatataggtctagaccttctaccgtctatccattaagttatgaagggtttctcaattgctatgaaggccgacagcgtactcgttacgcccaagccgtcgagcagttgatgcggtccactcttgagccgaaagatgcgcgagttgaaacgttcattaagaacgagaaatttgactgggcgttgaaaggggaggaggctgatcctcgagcaatccaaccaaggaagccgaaatatttggctgaggttggacggtggttcaaacctttggagcgaatcatctacaaggatctcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgtaagtctcccactctccagcgctatctcaaatggacactccgcaaccatggcgtcgcctcctgcaaagaattgtcatatgagtatgaggttgttggccggagaatgagtggtgacatggacactgcattgggcaactgcgtcattatgtcgatacttacatggtttatgcttagtgaacttggcattaagcatgaattattcgataatggtgacgattgtttgttcatttgcgagtctcacgacgtccccagccccgaggtaattacaaactggttttcggactttgggtttgtggttaggttggaaggcgtcacgtccgtgtttgagcgtattgagttttgccaaacttccccagtatggactgagaggggttggctgatgtgtaggaatattaagtcattgagtaaagaccttacgaatgttaattcgtgcacgggctccacgattgaatatacccactggttgaaagcagtgggaaagtgcgggtcaatactcaatgctggtgtacctatatttcagtcctttcacaacatgctggaaaggcttggcactaactctcgtattgatcgaggggttttcttcaaatcagggctagttaatctcattcgtgggatggacaggcagcctgacgttgacatcactacttccgctcggctttctttcgaagtggcattcgggataacacccgggatgcaattggctattgaacggtactatgactctgtcatgggctcgctgagtaaaatagaaacaactaagtggccaattgaactaagaaaggaatacgaacacggaagtgagtggtacgaggacttaggcgtcctaggatgaatagggtcattggtttaccgatgatacctgttcagaataggattgctcgagcttcgttggttagggtaactcacataccttcttccataactggaaaaggtcgtgtgagcaacctaaccagttaatgtaggtgtctttccgtatctagtcacgatggtaagcaacccgtttatctgtacggcgctcacccgtgggtaggaaggtgaaggttttgtgtcctttaggtcttggacagtctgcgggcttgggaacgacgccccgctagcaacgtactgctctcctaccggactggtagcttaattgtcatcttggagcgatagcactgtgggcctcacccttcgcgcgttggacgtgttgcgtgccccccacagatttgtgaaactctatggagcagttccgcgagccagaagggaggatggccgcctggcgtaatccaggagctctggggggcttgtactcagagtagcattctgctttagactgttaactttatgaaccacgcgtgtcacgtggggagagttaacagcgccc

SEQ ID NO: 2 - protein, 190 aa, synthetic construct
MDPSSLPRACWHHARQVFRASTSFSREVVRAARRWVTMPTSRHAYDFSTSLGIVIAEPAARLRRRLPSVRKCAEKLVVHKQVDTLVDEWCSGIPNPDIVEVGWALRLRDRFGLPPASEPTRLSGERWVLKQLNGVDPESWNADLGRSVHIQGDYAPGRNAHIAQVAATLWLTRTLHDKALARHQGFRDLQ

SEQ ID NO: 3 - protein, 469 aa, synthetic construct
MTTRVLQYKGRDPILPSSEALHRLNLRIAELYRSRPSTVYPLSYEGFLNCYEGRQRTRYAQAVEQLMRSTLEPKDARVETFIKNEKFDWALKGEEADPRAIQPRKPKYLAEVGRWFKPLERIIYKDLSKRLYGEGAEPCIAKGLNALESGATLRRKWEKFSSPVCVSLDASRFDLHVSVGMLKFTHKLYDYYCKSPTLQRYLKWTLRNHGVASCKELSYEYEVVGRRMSGDMDTALGNCVIMSILTWFMLSELGIKHELFDNGDDCLFICESHDVPSPEVITNWFSDFGFVVRLEGVTSVFERIEFCQTSPVWTERGWLMCRNIKSLSKDLTNVNSCTGSTIEYTHWLKAVGKCGSILNAGVPIFQSFHNMLERLGTNSRIDRGVFFKSGLVNLIRGMDRQPDVDITTSARLSFEVAFGITPGMQLAIERYYDSVMGSLSKIETTKWPIELRKEYEHGSEWYEDLGVLG

SEQ ID NO: 4 - RNA, 166 nt, unidentified
  source 1..166: mol_type = viral cRNA; note = CY1
tcgctcaggtcgcggcgaccttgtggttaactaggaccttgcatgacaaggccttggctcgccaccagggttttcgcgatttgcagtgattggggtcgacgggctagaggcaaaagcagtgcctctagcttctggactccgactgcttccggttccgcgacccgga

SEQ ID NO: 5 - RNA, 25 nt, unidentified
  source 1..25: mol_type = viral cRNA; note = CY1
caaagtcgacgactgtctcagacct

SEQ ID NO: 6 - RNA, 34 nt, unidentified
  source 1..34: mol_type = viral cRNA; note = CY1
aggtcttggacagtctgcgggcttgggaacgacg

SEQ ID NO: 7 - RNA, 2983 nt, unidentified
  source 1..2983: mol_type = viral cRNA; note = Citrus yellow vein-associated virus
gggtaaatatggaatcttcatctttgccccgtgcctgctggcaaaaggccaggcaggtgttgcgagcaccagctaccttctcgagagaggtggtcaaagctgcccgcaaatgggtcaccatgcccagtaaccaccgtgtctacgactactccactagtctgggcattgtgattgctgagcctgtggctcgtctgcgacgcagtctaccctccgtacgaaagtgcgcagagaagatagtagtccaccagggaatcgacaccttggtggacgattggtgtacaggacttcctaaccctgatatagtggaagttggttgggcactccgtctgagggaccgtttcggtcttcctcccgcctctgagcctactaggcttagtggtgagagatgggtgctcaaacaactcaatggggtagatcccgagtcatggaatgctgatctgggtaaaccagttcacgtccagggagactacgccccagggaggaatgcccacatcgcgcaggtcgcggcgaccttgtggctaactaagaccttgtctgacaaggccttggcccgccaccagggttttcgcgatttgcagtgattggggtcgacgggctagaggctaaagcagtgcctctagctgcaggactccgactgcttccggttccgcggcccggacaaagccgacggctgtctcagaccttgttactaccaacccctcgtgcacaatttgtgaatcacgcgaactcggctaataaccttgggcgcgtgatgaccacacgagtgatgcagtacaaaggccgagacccgatccaaccctctcaagaagccctaaccaaacttaaccttcggatagccgagctatataagtcgagaccatccaccgtctatccgttgagttatgaagggtttctcaactgctacgaaggccgacagcgtactcgttacgctcaagccgtcgagcagctgttgaggtccactctagaacctaaagatgcgagagttgaaacgttcattaagaacgagaagtttgattgggcgttgaaaggggaggaggctgatcctcgtgcaatccaaccaaggaagccgaaatatctggctgaggttggacggtggtttaaacctttggagcgaatcatctacaaggatcttagtaagcggttgtatggcgagggtggtgaaccttgtattgccaaaggcttaaatgcactagaatctggggcgactttgaggcgcaaatgggagaagttttcttctcctgtttgcgtctctcttgacgcttccaggttcgacctgcatgtaagtgttggcatgcttaagtttacacacaagctgtacgactactactgcaagtctcccaccctccaacgttatctcaaatggacactccgcaatcacggcactgcctcctgtaaagaattgtcatatggttatgaggtagaaggccgaagaatgagtggtgacatggacaccgcattgggcaactgcgtcatcatgtcgatactaacatggtttatgcttagtgagcttggcattaagcatgaattattcgacaatggtgacgactgcttgttcatttgcgagtcaaaagacgtccccagccccgaggtgatcacgaactggttttcggactttgggtttgtggtcaggttggaaggcgtcacgtccgtgtttgagcgtatagagttttgccaaacttccccagtatggactgagaggggttggctgatgtgtaggaacattaagtcgttgagtaaggaccttacgaatgtcaattcgtgcacgggctccacaattgaatatactcactggttaaaagccgtggggaagtgcgggtcaatactcaatgctggtgtgcctatatttcagtcctttcacaacatgctggaaaggcttggtactaactctcgtatagatcgcggggtgttctttaaatctgggctagtcaatctcattcgtgggatggacagacaacctgacgttaagatcaccacttccgctcgtctttctttcgaagtggcattcgggattacacctggaatgcaattggctattgaacggtactatgactcagtcatgggcccgctgggtaaaatagaaacaactaaatggccaatagaactaagaaaggaatacgagtacggaagcgagtggtacgaagacttaggcgtcctaggatgagcagggacatcggtttaccgatggcccccgttcagaataggatggctagggcttcgttggttagggaaacctatataccttcttccaccacaactggtaaggaggctgtgtgggcaacctggccaattaatgtaggtgtctttccgcaactagccacgatagtaagcaacccgttatactggcgtgtgaccagtgtgcaagtggcaatggagccagcaacgtcaacctctacccaactctgtggtgtaggtctcagtacatctggggcttacaactcaggggaagggttcgggaacacgtttaagctcttgcgctcttgtaactatacgagacgctcacccgtgggagggaacgtgatggtcaagtggcccatcagcatgccctacattcttaatgatgatgcgcacaagaccacaggtctaacagcttgtgtggtcattgctgtcaccaatccaggtgccatcactggccaatcttgggcggagattcaattgaatgtagagtacgtagtgggtacttagacagtctgcgggcttgggaacgacgccccgctagcaacgtactgctctcctaccggactggtagccgagctgttatcttggagcgatagcactgtgagcctcaccctgcgcgcgatggacgtgttgcgtgaccctcacagatttgtgaaactctatagagtagttccgcgagccagaagggaggatggcgacctggcgaaatccaggagctctggggggcttgtactcagagtagcaatactgctttagactgttagtttgatgaaccacgcgtgtcacgtggggagaactaacagcgccc

SEQ ID NO: 8 - RNA, 167 nt, synthetic construct
  source 1..167: mol_type = other RNA; note = GFPmmck59 Inserted at CYVaV Position 2304
aaaaggtcgtgtgagcaacctaaccagttaatgtaggtgtctttccgtatgaagcggcacgacttcttcaagagcgataactcgccttgacagaagtccaacgcttcatctagtcaggatggtaagcaacccgtttatctgtacggcgctcacccgtgggtaggaag

SEQ ID NO: 9 - RNA, 61 nt, synthetic construct
  source 1..61: mol_type = other RNA; note = Mmck6.1
ggaagtgatggacgaaattaatgattccataactggaacattacatttcgtcaacacttcc

SEQ ID NO: 10 - RNA, 61 nt, synthetic construct
  source 1..61: mol_type = other RNA; note = CS7-V2.2
ggtatcaatgggcagacgaagatttggcataactgccaatcttccgtctgcaaatgatacc

SEQ ID NO: 11 - RNA, 155 nt, synthetic construct
  source 1..155: mol_type = other RNA; note = F3-C3#3
actagaggaagtgttgacgaaatgtaatgttccagttatggaatcattaatttcgtccatcacttcctacggaaagacacctacattaactggtatcatttgcagacggaagattggcagttatgccaaatcttcgtctgcccattgataccaac

SEQ ID NO: 12 - RNA, 83 nt, synthetic construct
  source 1..83: mol_type = other RNA; note = Polynucleotide Insert into CYVaV
ttggttagggtaactcacataccttcttccatatgaagcggcacgactatctagtcannntggtaagcannncgtttatctgt

SEQ ID NO: 13 - RNA, 240 nt, synthetic construct
  source 1..240: mol_type = other RNA; note = Polynucleotide Sequence of CBC460, CBC461 and CBC462
taaccagttaatgtaggtgtctttccgtatctagtcacgatggtaagcaacccgtgcgtgttacgtcagtgacaatcgccatggaaccggcaatgtctacctctacccaacactgtggggtaggactcgctactagcggggcctactcttcaggtgagggttttggtaacttgttcaagtacaagcgcgcttgtaactttatctgtacggcgctcacccgtgggtaggaaggtgaaggtt

SEQ ID NO: 14 - RNA, 144 nt, synthetic construct
  source 1..144: mol_type = other RNA; note = Polynucleotide Sequence of OULV-insert
tgcgtgttacgtcagtgacaatcgccatggaaccggcaatgtctacctctacccaacactgtggggtaggactcgctactagcggggcctactcttcaggtgagggttttggtaacttgttcaagtacaagcgcgcttgtaact

SEQ ID NO: 15 - RNA, 240 nt, synthetic construct
  source 1..240: mol_type = other RNA; note = Polynucleotide Sequence of CY2220 (1132-1329)2280-1 and CY2220 (1132-1329)2280-2
attgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgt

SEQ ID NO: 16 - RNA, 198 nt, synthetic construct
  source 1..198: mol_type = other RNA; note = Wt:29/44
cagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactg

SEQ ID NO: 17 - RNA, 300 nt, synthetic construct
  source 1..300: mol_type = other RNA; note = Polynucleotide Sequence CBC135
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 18 - RNA, 300 nt, synthetic construct
  source 1..300: mol_type = other RNA; note = Polynucleotide Sequence CBC139
ggtcattggtttaccgatgatacctgttcagaataggattgctcgagcttcgttgcagtaaaaggttgtatggtgatggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 19 - RNA, 300 nt, synthetic construct; identical to SEQ ID NO: 18
  source 1..300: mol_type = other RNA; note = Polynucleotide Sequence CBC140

SEQ ID NO: 20 - RNA, 300 nt, synthetic construct
  source 1..300: mol_type = other RNA; note = Polynucleotide Sequence CDE967
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgttcctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 21 - RNA, 300 nt, synthetic construct
  source 1..300: mol_type = other RNA; note = Polynucleotide Sequence CDE974
ggtcattggtttaccgatgatacctgttcagaataggattgctcgagcttcgttgcagtaaaaggttgtatggtgagggtgctgagccgtgtatcgccaaaggcctaaatgcattacaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 22 - RNA, 300 nt, synthetic construct
  source 1..300: mol_type = other RNA; note = Polynucleotide Sequence CDE977
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagcttgcgtttccctcgacgcttccaggttcgacctgcatgtaagcgttggcatgctaaagctcacacacaagctatatgactattactgcagttaatgtaggtgtctttccgtatctagtcacgatggtaagcaac

SEQ ID NO: 23 - RNA, 300 nt, synthetic construct
  source 1..300: mol_type = other RNA; note = Polynucleotide Sequence CDE978
ggtcattggtttaccgatgatacctgttcagaataggattgctcgagcttcgttgcagtaaaaggttgtatggtgagggtgctgagccgcgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 24 - RNA, 300 nt, synthetic construct
  source 1..300: mol_type = other RNA; note = Polynucleotide Sequence CDE980
ggtcattggtttaccgatgatacctgttcagaataggattgctcgagcttcgttgcagtaaaaggttgtatggtgagggtgctgagccgtgtatcgccaaaggtctaaatgcattagaatctggagcgactttgaggcgcaaatgggagaagttttcttctccagcttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 25 - RNA, 300 nt, synthetic construct
  source 1..300: mol_type = other RNA; note = Polynucleotide Sequence CDF015
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcatacacaagctatatgactattactgcagttaatgtaggtgtctttccgtatctagtcacgatggtaagcaac

SEQ ID NO: 26 - RNA, 300 nt, synthetic construct
  source 1..300: mol_type = other RNA; note = Polynucleotide Sequence CDF016
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcaacgcttccaggttcgacctgcatgtaagcgttggcatgctaaagttcacacacaagctatatgactattactgcagttaatgtaggtgtctttccgtatctagtcacgatggtaagcaac

SEQ ID NO: 27 - RNA, 300 nt, synthetic construct
  source 1..300: mol_type = other RNA; note = Polynucleotide Sequence CDF017
ggtcattggtttaccgatgatacctgttcagaataggattgctcgagcttcgttgcagtaaaaggttgtatggtgagggtgctgagccgtgtatcgccaaaggcctaaatgtattagaatctggagcgactttgaggcgcaaatgggagaagttttcttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 28 - RNA, 300 nt, synthetic construct
  source 1..300: mol_type = other RNA; note = Polynucleotide Sequence CDF020
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcacgtaagcgttggcatgctaaagttcacacacaagctatatgactattactgcagttaatgtaggtgtctttccgtatctagtcacgatggtaagcaac

SEQ ID NO: 29 - RNA, 300 nt, synthetic construct; identical to SEQ ID NO: 20
  source 1..300: mol_type = other RNA; note = Polynucleotide Sequence CDF037

SEQ ID NO: 30 - RNA, 300 nt, synthetic construct
  source 1..300: mol_type = other RNA; note = Polynucleotide Sequence CDF041
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatacgactattactgcagttaatgtaggtgtctttccgtatctagtcacgatggtaagcaac

SEQ ID NO: 31 - RNA, 300 nt, synthetic construct
  source 1..300: mol_type = other RNA; note = Polynucleotide Sequence CDF047
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagttttcttctccagttcgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 32 - RNA, 300 nt, synthetic construct
  source 1..300: mol_type = other RNA; note = Polynucleotide Sequence CDF049
ggtcattggtttaccgatgatacctgttcagaataggattgctcgagcttcgttgcagtaaaaggttgtatggtgagggtgctgagccgtgtatcgccaaaggcctaaatgcattagaatctggagcgactttgaggcgcaaatgggagaagtttccttctccagtttgcgtttctctcgacgcttccaggttcgacctgcatgtaagcgttggcatgctaaagttcacacacaagctatatgactattactgcagttaatgtaggtgtctttccgtatctagtcacgatggtaagcaac

SEQ ID NO: 33 - RNA, 61 nt, synthetic construct
  source 1..61: mol_type = other RNA; note = Mmck6.2
ggtttcagtatatttcgtcatcaccttcctaacggaagtgatggacgaaattaatgaaacc

SEQ ID NO: 34 - RNA, 58 nt, synthetic construct
  source 1..58: mol_type = other RNA; note = Insertion of Mmck6.2 into CYVaV1
gtttcagtatttcgtcatcaccttcctaacggaagtgatggacgaaattaatgaaacc

SEQ ID NO: 35 - RNA, 60 nt, synthetic construct
  source 1..60: mol_type = other RNA; note = WT Insert Structure
ggttagggtaactcacataccttcttccataactggaaaaggtcgtgtgacaacctaacc

SEQ ID NO: 36 - RNA, 61 nt, synthetic construct
  source 1..61: mol_type = other RNA; note = Mmck15 Insert
gtcgcaatcaaagacgaagaaatcgtccataactggacatttccttcgtctcaattgcgac

SEQ ID NO: 37 - RNA, 61 nt, synthetic construct
  source 1..61: mol_type = other RNA
ggttgcttggaacccatacgaatgttgcttaacagcaaattcgctatgggtgatagcaacc

SEQ ID NO: 38 - RNA, 61 nt, synthetic construct
  source 1..61: mol_type = other RNA; note = MmckpsvD Insert
gggtgaagtcatgaaagaagctgccttcttaacagaagcagctctctttcacaattcaccc

SEQ ID NO: 39 - RNA, 61 nt, synthetic construct
  source 1..61: mol_type = other RNA; note = MmckpsvE Insert
ccgtaggtaagacaccattgacaagttcttaacagaactgtcacatggtgtgaccctacgg

SEQ ID NO: 40 - RNA, 61 nt, synthetic construct
  source 1..61: mol_type = other RNA; note = PRSVmmck1 Insert
atggtctgaatgataatgaaatgcaagtgtaaccacttcatttccattatccaaagaccat

SEQ ID NO: 41 - RNA, 61 nt, synthetic construct
  source 1..61: mol_type = other RNA; note = PRSVmmck2 Insert
gcagaagcatatattgcaaagagaaatgctaacgcattctcttctgcaatacaacttctgc

SEQ ID NO: 42 - RNA, 199 nt, unidentified
  source 1..199: mol_type = viral cRNA; note = Citrus yellow vein-associated virus [CYVaV]; note = Wild Type Structure 1 (1132-1329) of CYVaV
cagtaaaaggttgtatggtgagggtgcttgagccgtgtatcgccaaaggcctaaatgcattagaatctggagcgactttgaggcgcaaatgggagaagttttcttctccagtttgcgtttctctcgacgcttccaggttcgacctgcatgtaagcgttggcatgctaaagttcacacacaagctatatgactattactg

SEQ ID NO: 43 - RNA, 198 nt, synthetic construct
  source 1..198: mol_type = other RNA; note = PDS-198mmck-1 Insert
cacgaaacagaagtacttggcttcaatggaaggtgctgtcttatcaggaaagctttgtgcacaagctattgtacaggattacgagttacttcttggcttttgccaagatgtaactcgtaaacaaattacaaatagcttgtcaaacaaaactgtcggataaacagtaaacttccttgcctacaagtaagtctattcgtg

SEQ ID NO: 44 - RNA, 198 nt, synthetic construct
  source 1..198: mol_type = other RNA; note = PDS-198mmck-2 Insert
gttgctcagtgtgtacgctgacatgtctgttacatgtaaggaatattacaaccccaatcagtctatgttggaattggtatttgcacccgcagaagagttttctcttctacgggtgcaaataagtgtttcacaacatagaaaatattggagtcgtctattcttacccaataacaacaacaaagcgtacacacaagcaac

SEQ ID NO: 45 - RNA, 198 nt, synthetic construct
  source 1..198: mol_type = other RNA; note = LcrGyrA-Mmck5 Insert
acctggtactgttcggcgaaataaattatctgattttgtgcatgtgaaccgtaatggtaagattgcaatgaaattggaggaaaatgatgagattgttttttaacaatcgcatcattttccccattcgctcatgtgcaatgaccaccatcgttgtaacatgacaactcacagataataccatcgccgaacaccccaggt

SEQ ID NO: 46 - RNA, 198 nt, synthetic construct
  source 1..198: mol_type = other RNA; note = LcrGyrA-Mmck3 Insert
atgatgagattgtttcagtagaaacttgtactgaggaccatgatgttttgctaacaacagaatttggtcagtgtattcgattcccagtttctaatgtttttacattagcaactgtgaatcccacacacctgacccatttaaacgttgtcaaaaaccattaggtcaaagagtacagtggaaactgaaacaacccatcat

SEQ ID NO: 47 - RNA, 198 nt, synthetic construct
  source 1..198: mol_type = other RNA; note = Clas-GyrAmmck-5 prime Insert
gcatggcaatgtacggcgtaataaactttctgattttattcaaatcaatcgtagtggtaagattgcgatgaaattagattcaagagatgagattcttttttaagaatcccatctcttgaaacattccctcatgcgcaataaccaccacccatattatttgataacccacagaaggttcaaatgctgtacaccccatgc

SEQ ID NO: 48 - RNA, 198 nt, synthetic construct
  source 1..198: mol_type = other RNA; note = Clas-GyrAmmck-3 prime insert
gagatgagattctttccgttgaaacctgtacacaagaaaatgatatattgttgactactaaacttggacaatgtgtccgctttccgatttctgctatttttatagcagcaatcggaaagctaacacacttgtcccaagtggccgtagttccaaactatcatttcgccctgtacggtaaacacggaaagaaaacatctc

SEQ ID NO: 49 - RNA, 198 nt, synthetic construct
  source 1..198: mol_type = other RNA; note = CS7 Insert
agaattatggagcggtcaagctcacacatcgtcacactttttatgtggtgggctcagccaaaactgtatgtaggaagaggcttgcatgagggcatgtttttacatgccatcatgcaagccagacgactatcatacagttgaccgctgatccaactcataaaagtcaaaatgattgtacaattgattgctctcaattct

SEQ ID NO: 50 - RNA, 198 nt, synthetic construct
  source 1..198: mol_type = other RNA; note = Erwinia GyrA Insert
ccgaccgcgcagcgccggtattattgccgtcaatctgcgtgatgacgatgaactgatcggcgtgtcgctgacgaacggtagtgatgaagcgatgctgttttcagcatcacttcatcactaaaaagacgtacagtgacacataagatcacttaatatcatccgtatacatgatgcaaaccaattggtgttgtaggttgg

SEQ ID NO: 51 - RNA, 66 nt, synthetic construct
  source 1..66: mol_type = other RNA; note = Hairpin Targeting GFP (CYVaV-GFPhp2301)
tgaagcggcacgacttcttcaagagcgccagaattctggcgctcttgaagaagtcgtgccgcttca

SEQ ID NO: 52 - RNA, 65 nt, synthetic construct
  source 1..65: mol_type = other RNA; note = M2250GFP30ext
ggttagggtaactcactgaagcggcacgacttcttcaagagcgccattcagtgagcaacctaacc

SEQ ID NO: 53 - RNA, 59 nt, synthetic construct
  source 1..59: mol_type = other RNA; note = GFPmmck59
tgaagcggcacgacttcttcaagagcgataactcgccttgacagaagtccaacgcttca

SEQ ID NO: 54 - RNA, 63 nt, synthetic construct
  source 1..63: mol_type = other RNA; note = GFPmmck63
tgaagcggcacgacttcttcaagagcgccataatcggcgccttgacagaagtccaacgcttca

SEQ ID NO: 55 - RNA, 128 nt, Citrus tristeza virus
  source 1..128: mol_type = viral cRNA; note = CTV-insert-natural-V2.2
gggggtttatgtttggcaaagaaagtgttggaactgttagtcaagcgggtggttgaatcgttttctcgtttgaagcggaaaaccgctcgtttaacgtccttcgctaatttgttgcttgcgaggctctc

SEQ ID NO: 56 - RNA, 138 nt, synthetic construct
  source 1..138: mol_type = other RNA; note = CYVaV1 with Insert-Natural-V2.2
tcgttgggggtttatgtttgcaaagaaagtgttggaactgttagtcaagcgggtggttgaatcgtttctcgtttgaagcggaaaaccgctcgtttaacgtccttcgctaatttgttgcttgcgaggctctcagttaat

SEQ ID NO: 57 - RNA, 198 nt, synthetic construct
  source 1..198: mol_type = other RNA; note = BBLv2-1
ctggaaaattcgattactcaaagtgcctaaaatcgtaacaacttgtatgatctataggcctggatccaaacaatacgtggctcaaatacttgagaaatttttttctcatgtatttgagcctaaaccttgatttggatccatctcctatcgaacaccaagtgttaaactttttagcagagggagtaatcgaaattccag

SEQ ID NO: 58 - RNA, 37 nt, synthetic construct
  source 1..37: mol_type = other RNA; note = BBLv2-1
ctggaaaattcgattactcaaagtgtcgaaattccag

SEQ ID NO: 59 - RNA, 743 nt, Erwinia amylovora
  source 1..743: mol_type = transcribed RNA; note = Polynucleotide Encoding Erwinia amylovora MurA
gaactggtgaaaaccatgcgcgcctcgatttgggcattgggcccgctggtggcacgttttggccaggggcaagtatcactgcccggtggttgcgctatcggcgcacggccggttgatcttcatatcaccggccttgagcagctcggcgccgagatcaaactggaagaaggttacgttaaagcctctgtcgcgggtcgcctgaaaggggcgcatatcgttatggataaggtcagcgtgggtgcaaccgtcactatcatgagtgcggcgacgctggcaacgggcaccaccgttatcgagaatgctgcgcgtgagccggaaattgtcgacactgccaacttcctcaacacgcttggggcgaaaatcaccggtgccggcagcgatcgtatcaccatcgaaggtgttgatcgccttggtggcggtgtttatcgcgtacttcctgaccgcatcgaaaccggtactttcctggtggcgggagcgatttccggcggtaaggttacctgccgtgcggcgcagcccgatacgctggatgctgtactggctaagctacgcgaagccggtgcggacatcgagatgggagaagactggataagcctggacatgcacggtaagcggcctaaagcggtcaatttacgcacagcgccgcatcccggtttcccaaccgatatgcaggcgcagttcagtttgttgaacctggtggctgaaggcacgggggtgattactgaaaccatcttcgaaaaccgctt

SEQ ID NO: 60 - RNA, 607 nt, Erwinia amylovora
  source 1..607: mol_type = transcribed RNA; note = Polynucleotide Encoding Erwinia amylovora GyrA
atcgtcaacctgctgccgctggaagccaacgagcgtattactgcaattctgccggtgcgtgaatatgccgaaggctggaatatctttatggctaccgccagcggaacggtgaagaaaaccgcgctgactgacttcagccgaccgcgcagcgccggtattattgccgtcaatctgcgtgatgacgatgaactgatcggcgtgtcgctgacgaacggtagtgatgaagcgatgctgttttctgccgccggtaaagtggtgcgattcgcggagagcgcggtgcgtacgatgggccgtaccgcctccggcgtacgcggcatcaagctggctgaaggcgaccgcgtggtgtcgctgatcgtgccgcgtgatgatggcgctatcatgaccgtgacgcaaaacggctacggtaaacgaacggctaacgtcgaatatcccaccaagtcgcgtgcgactcagggggttatctcgatcaaggtaaccgagcgtaacgggccggttatcggtgcggtgcaggtggtcgatggcgatcagatcatgatgatcaccgatgccggcacgctggtacgtacccgcgtgtccgaggtcagcgtggtagggcgtaacacccagg

SEQ ID NO: 61 - RNA, 2808 nt, Pseudomonas syringae
  source 1..2808: mol_type = transcribed RNA; note = P. syringae tabaci_Gyrase subunit A (Pst_GY)
atgggcgaactggccaaagaaatcctcccggtcaatatcgaagacgagctgaagcagtcctacctcgactacgcgatgagcgtaatcgtcggtcgagcactgcccgatgcgcgcgacggcttgaagcccgtgcaccggcgcgtgttgttcgcaatgagcgagctgggtaacgactggaacaagccgtacaagaaatccgcccgtgtggttggtgacgtgatcggtaagtatcacccgcacggcgatacagccgtgtacgacaccatcgttcgtatggctcagccattctcgctacgctacctgctggtagacggtcagggcaacttcggttcggtcgatggcgacaacgctgcggccatgcgatacaccgaagtgcgcatgaccaagctggcgcacgagctgctggccgacctgcacaaggaaaccgtggactgggtgccgaactacgacggcaccgaaatgatccccgcggtcatgccgacccgtattcccaacctgctggtcaacggttccagcggtatcgccgtgggcatggcgaccaacattccgccgcacaaccttggtgaagtcatcgacggttgcctggcactgatcgacaaccccgagttgacgatcgatgagctgatgcagtacatccccggcccggatttcccgacagcggcgatcatcaacggccgtgcgggtatcatcgaagcctatcgtacgggtcgtggacgcatttacatgcgtgcccgctccatcgtcgaagacatcgacaaggtcggcggacgtcagcagatcgtcatcaccgaactgccgtaccagcttaacaaggcacgtctgatcgagaagatcgccgagctggtcaaggaaaagaaactcgaaggcattaccgagctgcgtgacgagtccgacaaggacggtatgcgcgtggtcatcgagctgcgtcgtggcgaagtgccagaggttgttctcaacaacctttatgcccagacccagctgcaaagcgttttcggtatcaacatcgttgccttgattgacggtcgtccacgcatcctgaacctcaaggacctgttggaagcctttgttcgtcaccgtcgcgaagtggttacccgccgtaccgtattcgagctgcgcaaggcgcgtgagcgtggccatatccttgaaggtcaggctgttgcgttgtccaacatcgacccggtcatcgccctcatcaaggcctctccgacacctgcagaagccaaggaggcgttgatcaagacgccttgggaatcaagcatgcgcgtgattctgctaggagcagtggtcgaaatggtcgagcgtgcaggtgccgattcgtgccgccctgagaacctggacccgcaatacggtctgcgtgaaggcaagtatttcctgtcaccggaacaggctcaggccattctggaactgcgtctgcatcgcctgaccggtctggaacacgagaagctgctgggcgagtaccaggaaatcctcaaccagatcggcgagctgatccgcatcctcaacagcgcaacgcgcctgatggaagtgatccgcgaagagctggaagtgatccgctccgagtacggcgatgcccgtcgtaccgagattctcgatgcacgtctggacctgaccctgggtgacctgatcaccgaagaagagcgtgtggtcaccatctcccatggcggctatgccaagacccagccattggcggtctatcaggctcagcgtcgtggcggcaagggcaagtcggccaccggcatcaaggatgaggattacattgctcacctgctggtcgccaacagccacacgacactgctgatgttctccagcaagggcaaggtgtactggctcaagacctatgagatcccggaagcgtcccgcgctgcccgtggtcgtccgttggtcaacctgttgccgctgagcgacggcgaatacatcaccaccatgctgccggttgacctcgaagccatgcgcaagcgtgccgacgaagaaggcgaagccctcgaaggcgagctggacgacgcggaaaacagcagcgagaccgaagaagagcgcaaggcccgtatcaaggccgccgacaagaagaaggctccgttcatcttcatgtccaccgccaacggtaccgtcaagaagaccccgctggttgcattcagccgtcaacgcagttcgggcctcattgcccttgagctggacgagggcgacatcctgatctccgctgccattaccgatggcgaacaggaaatcatgctgttctccgatggcggcaaagtgacccgcttcaaggaatccgatgtgcgcgccatggggcgtaccgctcgcggcgtgcgtggcatgcgtctgccagaagggcaaaagctcatttcgatgctgatcccggaagaaggcagccagatcctcaccgcttccgagcgcggttacggcaagcgtacggccatttccgagttccccgagtacaagcgcggcggtcagggtgtcatcgccatggtcagcaacgagcgtaacggccgtctggttggcgcagttcaggtgcttgatggcgaagaaatcatgctgatttccgatcagggcacgctggtgcgtacccgggtgggcgaagtgtccagtctgggccgtaacactcagggtgtgaccctgatcaagctggccagcgacgagaaactggtcggtctggagcgtgttcaggagccgtcggaagtcgaaggcgaagagcttgaaggcgaagaagttatcgacggcgtgattgtcgatgccgctgaagctgaagtgggcgacgccggtgaagacctgcaagcggacgctgcgccagacgaagacgaaccgcagaactga

SEQ ID NO: 62 - RNA, 663 nt, Pseudomonas syringae
  source 1..663: mol_type = transcribed RNA; note = P. syringae tabaci_Adenylate Kinase (Pst-ADK)
atgcgcgtgattctgctaggagctcccggggccggtaaaggtactcaggcaaaattcatcactgaaaatttcggcatcccgcaggtttcgacaggcgacatgctgcgcgctgcagtcaaggctgaaaccgagcttggcctgaaggccaagagcgtcatggactcgggtggtctggtttccgatgacctgatcattggtctgatcaaggatcgtctggcccagccggattgtgcgaacggcgttctgttcgacggcttcccgcgcaccattcctcaggccgaagccctgttgaaagcaggtctggaaatcgaccacgtgctggaaatcgccgttgatgacgaagaaatcgtcaagcgcatgtcgggccgccgggttcacgaaggctctggtcgcatctatcacaccattttcaacccgccgaaagtcgagggtatcgatgatgtgactggtgaaccgctgttgcagcgcaaggacgacgtcgaagaaaccgtgcgtcatcgcctgtcggtctaccatgcccagaccaagccgctggtcgagttctacagcaagctggaagcaaagaacggcaagcccaagtgcagccatattccaggtgttggctcggttgaagaaatcactgcaaaagtactgaaagcactggagcctgaagcacagaagtaa

SEQ ID NO: 63 - RNA, 717 nt, unidentified
  source 1..717: mol_type = transcribed RNA; note = GFPuv
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatcagttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataa

SEQ ID NO: 64 - RNA, 80 nt, unidentified
  source 1..80: mol_type = viral cRNA; note = Citrus yellow vein-associated virus [CYVaV]; note = CY1132-1329 (structure 1)
cagtaaaaggttgtatggtgagggtgctgagccgtgtatcgccaaaggcctaaatgcattagaatctggagcgactttga

SEQ ID NO: 65 - RNA, 33 nt, synthetic construct
  source 1..33: mol_type = other RNA; note = CY1493-1525 (structure 2)
tttatgcttagtgaacttggcattaagcatgaa

SEQ ID NO: 66 - RNA, 80 nt, synthetic construct
  source 1..80: mol_type = other RNA; note = CY2136-2218 (strucuture 3)
acgaggacttaggcgtcctaggatgaatagggtcattggtttaccgatgatacctgttcagaataggattgctcgagctt

SEQ ID NO: 67 - RNA, 61 nt, synthetic construct
  source 1..61: mol_type = other RNA; note = CY2220-2280 (structure 4)
ggttagggtaactcacataccttcttccataactggaaaaggtcgtgtgagcaacctaacc

SEQ ID NO: 68 - RNA, 51 nt, synthetic construct
  source 1..51: mol_type = other RNA
tgaagcggcacgacttcttcaatcaagagatgaagaagtcgtgccgcttca

SEQ ID NO: 69 - RNA, 61 nt, synthetic construct
  source 1..61: mol_type = other RNA
tgaagcggcacgacttcttcaagagcatcaagagagctcttgaagaagtcgtgccgcttca

SEQ ID NO: 70 - RNA, 32 nt, synthetic construct
  source 1..32: mol_type = other RNA
tgaagcggcacgacttcttcaagagcgccaga

SEQ ID NO: 71 - RNA, 32 nt, synthetic construct
  source 1..32: mol_type = other RNA
tctggcgctcttgaagaagtcgtgccgcttca

SEQ ID NO: 72 - RNA, 61 nt, synthetic construct; identical to SEQ ID NO: 67
  source 1..61: mol_type = other RNA

SEQ ID NO: 73 - RNA, 59 nt, synthetic construct; identical to SEQ ID NO: 53
  source 1..59: mol_type = other RNA

SEQ ID NO: 74 - protein, 21 aa, synthetic construct
GSGEGRGSLLTCGDVEENPGP

SEQ ID NO: 75 - RNA, 247 nt, synthetic construct
  source 1..247: mol_type = other RNA
acgaggacttaggcgtcctaggaggaagcggagagggcagaggaagtctgctaacatgcggtgacgtcgaggagaatcctggaccttttaggctaaagtttcatcaccaccaccatcaccactgaccggtgatagtgttggtgatgaaactttaggataaacgatccgatgttctaaccgaagtcaccgaaggccagcggaccccctctgacctacctgattcctgataggattgctcgagcttcgt

SEQ ID NO: 76 - protein, 33 aa, synthetic construct
GSGEGRGSLLTCGDVEENPGPFRLKFHHHHHHH

SEQ ID NO: 77 - RNA, 198 nt, synthetic construct
  source 1..198: mol_type = other RNA
acctggggatgacctcttttggtttaatcgtcctcgtctcattctttatctcattaattttgtcctctttcagaatgcttttcaattggccttctttttttaaagaagcccaattgaaaacttaaactgtaaagaggactcttattaaggaaattagaatagaccaccgacgataagagtaaagaggtcatgccaggt

SEQ ID NO: 78 - RNA, 198 nt, synthetic construct
  source 1..198: mol_type = other RNA
atgacctcttttggtttaatcgtcctcgtctccttctttatctcattaattttgtcctctttcagaatgcttttcaattggccttctttgcttggacttttgtccaagaaaagaaggccaccacacaaatgcattctgacacaaggaccaacttctgagaaaagctcagagacaggcaacttaaaccaaaacggtcat

SEQ ID NO: 79 - RNA, 198 nt, synthetic construct
  source 1..198: mol_type = other RNA
gttgattacttaaccctaaggcatggatttatcatcgcgcatttggcacctcagagccacgtaaagtttgatttccaaaagtatatcaacaggtcacttttgtgacctattgatatacttcagactaatacaaactttaaaccggctcagacgtacaaatcgcgccaaataaaccaacaattagggttaagcatcaac

SEQ ID NO: 80 - RNA, 202 nt, synthetic construct
  source 1..202: mol_type = other RNA
ggcctcaatttaccagtagaagagaaactaaaattatattcagatgatccttcaaagaccatgagattatcaacaagttttaatgttaggaaggagacatttttgtctccgtcctaacattatgttaatgtatgataatctggcgggtctgtgtagttcatcgaatccccattttgttaaaattctactggtacttgaggcc

SEQ ID NO: 81 - RNA, 198 nt, synthetic construct
  source 1..198: mol_type = other RNA
ctctagagaagtatgctcctgaatggccttctaatccatcatctttcagggaaatcgtgagcagatattgcagggaaattcgtcaactcggatttagttttctaaatcggagttgacgaaccaaaactgccaatatctgaaaaacgatatcacttaagatatggtaagagaagccaaaatggagcatatttactagag

SEQ ID NO: 82 - RNA, 198 nt, synthetic construct
  source 1..198: mol_type = other RNA
gccatagcagaaagcctggggttagataaagagtgtataaaagatgtattgggtgaacaaggacaacatatggctatcaattattatcctccttgtcttttgacaaggtggataataattctccctccattatgttgtcaaaagttcagccgatgatcattataattatcttttctccaaccaggctttctatatggc

SEQ ID NO: 83 - RNA, 199 nt, synthetic construct
  source 1..199: mol_type = other RNA
gacccaaatgcccttaccattttattgcaagatttagaagtggcaggtcttcaagttctcaaagacgacaaatgggttgctgttaatccccttcctatttttaggaagcggattaacagctttgaaattatgttgtcttccttgaattcgatgaatgtcattctttcttcttgaaatctcctggtaagggtaatgggtc

SEQ ID NO: 84 - RNA, 199 nt, synthetic construct
  source 1..199: mol_type = other RNA
cctcaacatggttgaagggagtaaaagatttttcgagctccctttcgaggagagatccaagtacatgtcgactgatatgtatgccccagttaggtatttttatatctatctggggtatacccacaaagtacgatatgtaaaacggatcactactaaaaggagctaattaaaatatttgggacccttcaaccacttgagg

SEQ ID NO: 85 - RNA, 197 nt, synthetic construct
  source 1..197: mol_type = other RNA
cttccacatgaggagagagctaagtacatgtcctctgatatgaaagctaaagtgcgatatggaacaagctttaaccaaactaaagacaaagtcttttttttaaaagacattgtctttagtaacccataaagcttgttcttaaatcgctctataatttcaatcacctcgacattacaatcctctctcctcaatggaag

SEQ ID NO: 86 - RNA, 224 nt, synthetic construct
  source 1..224: mol_type = other RNA
acaatcacagattgttatcataaagcgaattctttggttatctagctgtatgaatcacatcatccagcccgataaccaaagattgagtgtgtttactctaagtacaatttcaacagttatttcaatcagacaattgtatcataatgggtgctcaggtttcatcacagaaagtgggcgcacatgaaaactcaaatagagcgtatggtggttctaccattaattac

SEQ ID NO: 87 - RNA, 152 nt, synthetic construct
  source 1..152: mol_type = other RNA
attggccatccggtgacctctcccaacccttgtaccagtgtatatcactagcacaagcattgggagcgtttataccacttagcttgaaagaggttaaaacattacaattcattgttaagttgaatacagcaaaatgggagctcaagtatcaa

SEQ ID NO: 88 - RNA, 66 nt, synthetic construct; identical to SEQ ID NO: 51
  source 1..66: mol_type = other RNA; note = Hairpin Targeting GFP (CYVaV-GFPhp2301)

SEQ ID NO: 89 - RNA, 21 nt, synthetic construct
  source 1..21: mol_type = other RNA
tccgtggacgtcatgtgtaag

SEQ ID NO: 90 - RNA, 24 nt, synthetic construct
  source 1..24: mol_type = other RNA
ggaagtgatggacgaaattaatga

SEQ ID NO: 91 - RNA, 61 nt, synthetic construct; identical to SEQ ID NO: 33
  source 1..61: mol_type = other RNA; note = Mmck6.2

SEQ ID NO: 92 - RNA, 75 nt, synthetic construct
  source 1..75: mol_type = other RNA; note = Mmck6.2
ttcgttggtttcagtatatttcgtcatcaccttcctaacggaagtgatggacgaaattaatgaaaccagttaatg

SEQ ID NO: 93 - RNA, 80 nt, synthetic construct
  source 1..80: mol_type = other RNA; note = Mmck6.2
agcttcgttggtttcagtatatttcgtcatcaccttcctaacggaagtgatggacgaaattaatgaaaccagttaatgta

SEQ ID NO: 94 - RNA, 83 nt, synthetic construct
  source 1..83: mol_type = other RNA
acgaggacttaggcgtcctaggatgaatagggtcattggtttaccgatgatacctgttcagaataggattgctcgagcttcgt

SEQ ID NO: 95 - RNA, 174 nt, synthetic construct
  source 1..174: mol_type = other RNA
acgaggacttaggcgtcctaggaggaagcggagagggcagaggaagtctgctaacatgcggtgacgtcgaggagaatcctgaccttgacgatccgatgttctaaccgaagtcaccgaaggccagcggaccccctctgacctacctgattcctgataggattgctcgagcttcgt

(19) United States
(12) Patent Application Publication
(10) Pub. No.: US 2026/0275366 A1
(43) Pub. Date: Sep. 17, 2026

(54) RNA VECTORS WITH HAIRPIN-LIKE INSERTS

(71) Applicants: UNIVERSITY OF MARYLAND, COLLEGE PARK, College Park, MD (US); SILVEC BIOLOGICS INC., Gaithersburg, MD (US)

(72) Inventors: Anne Elizabeth SIMON, Upper Marlboro, MD (US); Chanyong JANG, Greenbelt, MD (US); Elizabeth CARINO, Alexandria, VA (US); Feng GAO, Gaithersburg, MD (US); George BELOV, Rockville, MD (US); Anna ZIMINA, College Park, MD (US); Stephen Xiaohua YANG, Cary, NC (US)

(21) Appl. No.: 19/480,296

(22) PCT Filed: May 3, 2024
(86) PCT No.: PCT/US2024/027884

§ 371 Date: Oct. 30, 2025

Related U.S. Application Data
(63) Continuation-in-part of application No. PCT/US2023/066542, filed on May 3, 2023.
(60) Provisional application No. 63/338,290, filed on May 4, 2022.
(60) Provisional application No. 63/343,168, filed on May 18, 2022.

(51) Int. Cl. C12N 15/82; C12N 15/86

(52) U.S. Cl. CPC C12N 15/8203; C12N 15/86; C12N 2770/32634; C12N 2770/32643; C12N 2770/32662

(57) ABSTRACT

The present disclosure relates to a viral vector having an exogenous RNA segment with a hairpin-like structure, for example having two or more base-paired regions separated by one or more non-base-paired regions. The exogenous RNA segment may have a secondary structure, minimum free energy, average positional entropy or other attributes within specified ranges, or with values similar to one or more hairpin-like structures of a reference wild type virus. In some examples, the viral vector is a live attenuated vaccine. In some examples, the viral vectors downregulates a susceptibility gene in a host plant.

### CROSS-REFERENCE TO RELATED APPLICATIONS

This application claims priority to International Application No. PCT/US2023/066542 (filed on May 3, 2023) which application is hereby incorporated by reference herein in their entirety.

### STATEMENT REGARDING FEDERALLY SPONSORED RESEARCH OR DEVELOPMENT

This invention was made with government support under 20207002933198 and 2022-70029-38492 awarded by the United States Department of Agriculture (USDA) and under MCB2034359 awarded by the National Science Foundation (NSF). The United States government has certain rights in this invention.

### REFERENCE TO SEQUENCE LISTING

This application includes one or more Sequence Listings pursuant to 37 C.F.R. 1.821 et seq., which are disclosed in computer-readable media (file name: 30059-P72448PC01_SequenceListing.xml, created on May 3, 2024, and having a size of 120,867 bytes), which file is herein incorporated by reference in its entirety. The sequences in the listing are RNA sequences, but use “t” to denote uracil pursuant to WIPO st.26 standards.

### FIELD OF THE INVENTION

The present disclosure relates to the field of RNA virus based vectors having an exogenous RNA segment. The vector may be suitable for introducing a therapeutic agent, such as a peptide, or a small RNA, into a host.

### BACKGROUND OF THE INVENTION

The following description is not an admission that anything described below or in the referenced publications is common general knowledge or citable as prior art in any country.

Both general and highly targeted anti-microbial agents have been developed for animals (e.g., humans) whose circulatory systems provide a delivery system for widespread application throughout the animal. In contrast, much less research has been conducted to develop general or targeted therapeutic agents for plants. Among other factors, the lack of an animal-like circulatory system complicates delivery throughout the host plant. This is especially problematic in large, long-lived trees (e.g., citrus), where injection of anti-microbial agents may be rapidly diluted. As a result, few solutions exist for treating plant infections or conditions beyond external application of pesticides to control the pathogen's vector, applications to strengthen a plant's health in general, or application of short-duration agents targeting the pathogen, or a vector of the pathogen.

Plant industries are at substantial risk from various pathogens. In the citrus industry, Huanglongbing (HLB), also known as Citrus Greening, is the most serious disease globally. HLB is associated with three species of the bacterium Candidatus Liberibacter spp. (asiaticus, africanus, and americanus) and is transmitted by two psyllid species, Asian citrus psyllid (ACP) (Diaphorina citri, Kuwayama) and African citrus psyllid (Trioza erytreae, Del Guercio). HLB is graft-transmissible and spreads naturally when a bacteria-containing psyllid feeds on a citrus tree and deposits the pathogenic bacteria into the phloem where the bacteria reproduce. The infected tree reacts by producing excessive callose and phloem protein 2 (PP2) in its phloem in order to isolate the bacteria, which restricts the flow of photoassimilates and can ultimately kill the tree. While the diseased fruit pose no health threat to humans, HLB has devastated millions of acres of citrus groves throughout the world. In the United States alone, ACP and CL asiaticus (CLas) have decimated the Florida citrus industry, causing billions of dollars of crop losses within a very short time span. Moreover, HLB has spread into every citrus producing region in the United States. Most infected trees die within a few years after infection, and fruit develops misshapen and off flavored years before tree death and is unsuitable for consumption. According to the United States Department of Agriculture (USDA), the entire citrus industry is at substantial risk.

Consideration of plant physiology aids in the development and implementation of strategies for managing plant diseases and conditions. The vascular system of plants is the key conduit for sugars and amino acids, as well as signaling molecules such as small ribonucleic acids (RNAs), messenger RNAs (mRNAs), proteins, peptides and hormones, which are required for a large number of developmental processes and responses to biotic and abiotic stress (Lee, J. Y. and Frank, M. (2018), Plasmodesmata in phloem: different gateways for different cargoes, Curr Opin Plant Biol 43:119-124; Tugeon, R. and Wolf, S. (2009), Phloem Transport: Cellular Pathways and Molecular Trafficking, Ann Rev Plant Biol 60:207-221). Many mRNAs comprise a portion of these transiting molecules, and thousands of companion cell mRNAs can be isolated from neighboring enucleated sieve elements, where they are transported bidirectionally by osmotically generated hydrostatic pressure from source (sugar generating) tissue to sink (sugar utilizing) tissue such as roots and shoot tips (Folimonova, S. Y. and Tilsner, J. (2018), Hitchhikers, highway tools and roadworks: the interactions of plant viruses with the phloem, Curr Opin Plant Biol 43:82-88; Ham, B. K. and Lucas, W. J. (2017), Phloem-Mobile RNAs as Systemic Signaling Agents, Annual Rev Plant Biol 68:173-195).

Plant viruses, many of which move through the plant as a ribonucleoprotein complex (vRNP), have evolved to use the same pathway as used by mobile endogenous RNAs. Plant viruses can accumulate in substantial amounts, and most initiate infection in epidermal or mesophyll cells and then move cell-to-cell through highly selective intercellular connectors called plasmodesmata, which allow for continuity between the cytoplasm of neighboring cells (FIG. 1; see also Lee, J. Y. and Frank, M. (2018), Plasmodesmata in phloem: different gateways for different cargoes, Curr Opin Plant Biol 43:119-124; Schoelz, J. E. et al. (2011), Intracellular transport of plant viruses: finding the door out of the cell, Mol Plant 4:813-831). Long-distance systemic movement (leaf-to-leaf) requires that the virus, in the form of RNA-protein complexes or RNA-encapsidated inside virions, enters companion cells, where replication takes place, followed by progeny exit into sieve elements by transiting through the specialized, branched plasmodesmata that connect companion cells and sieve elements. Once tubular sieve elements are reached, viruses move passively with the phloem photoassimilate stream and establish systemic infections upon exiting (Folimonova, S. Y. and Tilsner, J. (2018), Hitchhikers, highway tolls and roadworks: the interactions of plant viruses with the phloem, Curr Opin Plant Biol 43:82-88).

Delivering engineered therapeutic agents into plants is an established means of introducing traits such as resistance to pathogens or other desired properties into plants for research purposes. Various methods of providing vectors to plants are known in the art. This is often achieved by delivery of an RNA virus vector (virus engineered to contain exogenous RNA) into a plant cell's nucleus by Agrobacteria tumefactions-mediated “agroinfiltration,” which may result in a modification of that cell's genome, or by delivering the virus vector directly into a cell's cytoplasm, which results in infection without a requirement for plant genomic modification. In the case of agroinfiltration of RNA viruses, the cDNA of the viral genome is incorporated into the T-DNA, which Agrobacteria delivers into the plants. Such T-DNA includes further regulatory DNA components (e.g., promoter for cellular DNA-dependent RNA polymerase), which allow for transcription of the DNA to generate the full-length viral RNA genome within plant cells. The incorporated DNA version of the RNA virus that contains therapeutic DNA inserts is transcribed into the viral RNA within the plant cells, after which the virus behaves like a normal RNA virus (amplification and movement).

To act as an effective vector, a virus should be engineered to accept inserts without disabling its functionality and to ensure that the engineered virus is able to accumulate systemically in the host to a level sufficient to deliver and in some cases express peptides or proteins from the insert(s). These inserts, whether composed of open reading frames (ORFs) that will be translated into proteins, or non-coding RNA that will be used for a beneficial function, should be delivered into the targeted tissue in a manner that is effective and sufficiently non-toxic to the host or to any downstream consumption of the host, or to the environment. In addition, the virus vector must be non-toxic to the host plant and is preferably not capable of being transmitted from plant to plant. However, only a limited number of viral vectors exist that meet at least some of the above criteria and some are available for only certain plants (e.g., citrus tristeza virus for citrus) since most plant viruses have a very limited range of hosts. Moreover, maintaining stability of short sequences inserted into a vector, particularly for long periods (e.g., months or years), has raised numerous challenges. Conventional virus vectors quickly (i.e., within a few days or a few weeks) evolve or mutate, discarding all or part of the inserted sequences given that the native virus without inserts is generally more fit than the virus vector with the inserts.

CTV is a member of the genus Closterovirus. It has a flexuous rod-shaped virion composed of two capsid proteins with dimensions of 2000 nm long and 12 nm in diameter. With a genome of over 19 kb, CTV (and other Closteroviruses) are among the largest known RNA viruses that infect plants. Prior studies have demonstrated that CTV-based vectors can express engineered inserts in plant cells (U.S. Pat. No. 8,389,804; US 20100017911 A1).

International Publication Number WO 2020/102210, Plant Vectors, Compositions and Uses Relating Thereto, published on May 22, 2020, describes an RNA vector derived from citrus yellow vein associated virus (CYVaV) or one of its relatives.

International Publication Number WO 2021/097086, Plant Vectors, Compositions and Uses Relating Thereto, published on May 20, 2021, further describes an RNA vector derived from citrus yellow vein associated virus (CYVaV) or one of its relatives.

International Publication Number WO 2022/246005, Plant Vectors, Compositions and Uses Relating Thereto, published on Nov. 24, 2022, describes an RNA vector having an siRNA insert effective against a plant bacterial pathogen. The vector may be derived from CYVaV, a relative of CYVaV, or another plant virus such as tobacco rattle virus (TRV).

Relatives of CYVaV include other umbravirus-like associated RNAs (ulaRNA) as described in Structural Analysis and Whole Genome Mapping of a New Type of Plant Virus Subviral RNA: Umbravirus-Like Associated RNAs, Liu J. et al., Viruses 2021, 13, 646, which is incorporated herein by reference. The ulaRNAs identified in this paper are referred to as Group 2 umbravirus-like viruses (ULVs) in this specification.

Some relatives of CYVaV are also described in-1 Programmed ribosomal frameshifting in Class 2 umbravirus-like RNAs use multiple long-distance interactions to shift between active and inactive structures and destabilize the frameshift stimulating element, Mikkelsen A. et al., Nucleic Acids Res. 2023 Oct. 27; 51 (19): 10700-10718.

### SUMMARY OF THE INVENTION

This specification describes a ribonucleic acid (RNA) molecule comprising a segment derived from an RNA virus and one or more heterologous segments, wherein each heterologous segment comprises a) two or more base-paired regions, b) one or more non-base-paired regions separating the base-paired regions, and c) an apical loop at the end of one of the based paired regions and wherein said heterologous segment(s) are designed according to one or more parameters described herein, for example a minimum free energy and/or average positional entropy within defined limits. This specification also describes heterologous segments. This specification also methods of modifying an RNA molecule, for example a viral vector, by insertion of one or more heterologous segments.

In some embodiments, the heterologous segment has a minimum free energy in kcal/mol in a range of about −5 to +15, or in a range of about −10 to +10, from −0.44 multiplied by the number of bases of the heterologous RNA segment minus 1.89 or, if the heterologous RNA segment has more than 150 nucleotides, a minimum free energy in kcal/mol in a range of about −20 to +20 from −0.44 multiplied by the number of bases of the heterologous RNA segment minus 1.89.

In some embodiments, the heterologous segment has an average positional entropy (APE) less than 0.75, 0.65, 0.5, 0.4, 0.39, 0.36 or 0.32. In some embodiments, the APE is greater than 0.01, 0.02 0.03, 0.04, 0.05, 0.06 or 0.07. In some embodiments, the APE is from 0.01 to 0.75, 0.01 to 0.65, 0.01 to 0.5, 0.01 to 0.4, 0.01 to 0.39, 0.01 to 0.36 or 0.01 to 0.32. In some embodiments, the APE is from 0.02 to 0.75, 0.02 to 0.65, 0.02 to 0.5, 0.02 to 0.4, 0.02 to 0.39, 0.02 to 0.36 or 0.02 to 0.32. In some embodiments, the APE is from 0.03 to 0.75, 0.03 to 0.65, 0.03 to 0.5, 0.03 to 0.4, 0.03 to 0.39, 0.03 to 0.36 or 0.03 to 0.32. In some embodiments, the APE is from 0.04 to 0.75, 0.04 to 0.65, 0.04 to 0.5, 0.04 to 0.4, 0.04 to 0.39, 0.04 to 0.36 or 0.04 to 0.32. In some embodiments, the APE is from 0.05 to 0.75, 0.05 to 0.65, 0.05 to 0.5, 0.05 to 0.4, 0.05 to 0.39, 0.05 to 0.36 or 0.05 to 0.32. In some embodiments, the APE is from 0.06 to 0.75, 0.06 to 0.65, 0.06 to 0.5, 0.06 to 0.4, 0.06 to 0.39, 0.06 to 0.36 or 0.06 to 0.32. In some embodiments, the APE is from 0.07 to 0.75, 0.07 to 0.65, 0.07 to 0.5, 0.07 to 0.4, 0.07 to 0.39, 0.07 to 0.36 or 0.07 to 0.32.

In some embodiments, the base-paired regions of the heterologous segment have 19 or less base pairs or 17 or less base pairs or 13 or less base pairs or 10 or less base pairs.

In some embodiments, the heterologous segment has no more than 4 consecutive A:U base pairs.

In some embodiments, the heterologous segment has no more than 4, or no more than 3 consecutive G:C base pairs.

In some embodiments, the heterologous segment has no more than 21 or no more than 20 bases collectively on both sides in a non-base paired region of the heterologous segment.

In some embodiments, a) each base-paired region of the heterologous segment has an APE of less than 0.8 or less than 0.6 and/or b) the ΔG of a region including two non-base paired regions and one base-paired region between them is not positive.

In some embodiments, the heterologous segment has between 40 and 300, 40 and 200, 60 and 300 or 60 and 200 bases.

In some embodiments, the heterologous segment has a standard deviation of PE less than 0.5 or less than 0.4.

In some embodiments, less than 20% or 15% or less than 10% of the bases of the heterologous segment have a PE of greater than 1.0

In some embodiments, the largest PE of any base of the heterologous segment is not greater than 2.0 or not greater than 1.5.

In some embodiments, the heterologous segment replaces a hairpin-like region of the RNA virus.

In some embodiments, the heterologous segment has a minimum free energy within 10 kcal/mol, or within 5 kcal/mol of a naturally occurring hairpin-like structure of similar size, i.e. of a size within 10% of the size of the heterologous segment, or of a line of best fit between multiple wild type hairpin-like structures. Optionally, for long heterologous RNA segments, for example segments with 150 nucleotides or more, the heterologous segment has a minimum free energy within 20 kcal/mol of a naturally occurring hairpin-like structure of similar size, i.e. of a size within 10% of the size of the heterologous segment, or of a line of best fit between multiple haipin-like structures.

In some embodiments, the segment derived from the RNA virus has a sequence identity of at least 50%, at least 60%, at least 70%, at least 80%, at least 90% or at least 95% with the RNA virus. In some embodiments, the segment derived from the RNA virus is a viral vector or a live attenuated vaccine known in the art.

In some embodiments, the portion derived from an RNA virus is derived from a plant virus such as a ULV, CTV or TRV. In some embodiments, the portion derived from an RNA virus is derived from a Class 2 ULV. In one embodiment, the portion derived from an RNA virus is derived from CY1.

In some embodiments, the RNA virus is an animal virus. In some embodiments, the RNA virus is a live attenutated virus of a vaccine. In some embodiments, the RNA virus is an enterovirus, for example poliovirus or CVB3.

In some embodiments, the heterologous segment comprises an active portion (alternatively called a targeting portion). In some embodiments, the active portion comprises an RNA bioactive molecule, such as a sequence that generates one or more siRNAs. In some embodiments, the active portion is effective against a plant pathogen or suppresses expression of a gene in a plant. In some embodiments, the active portion targets a susceptibility gene of a host plant. In some embodiments, the active portion is complementary to a host microRNA. In some embodiments, the active portion encodes a peptide.

In some embodiments, the heteriologous segment has an active portion provided on a first side of the heterologous segment. For example the first side may have a coding or non-coding sequence that targets a pathogen or gene of a host. In some embodiments, a sequence on a second side of the heterologous segment is less than 90%, or less than 85%, but more than 65%, or more than 75%, complementary with the sequence of the first side.

In some examples, the RNO molecule, the heterologous segment or the method are not disclosed in U.S. provisional applications 63/338,290 or 63/343,168. In some embodiments, the heterologous segment is not a lock and dock and/or does not have a tertiary interaction with itself or with the RNA virus. In some embodiments, the heterologous segment is not attached to a truncated structure of the RNA virus. In some embodiments, the RNA virus is not a Class 2 ulaRNA.

This specification also describes a ULV, ulaRNA, Class 2 ULV, CY1, CTV or TRV vector comprising a heterologous RNA segment targeted against a host plant suspecptibility gene, wherein the heterologous RNA segment has one or more characteristics, for example an MFE and/or APE, of the heterologous segments described herein.

This specification also describes a live attenuated virus comprising a heterologous segment wherein the heterologous segment is complementary to a microRNA of the animal and has one or more characteristics, for example an MFE and/or APE, of the heterologous segments described herein.

### BRIEF DESCRIPTION OF THE DRAWINGS

The priority application of the present application contains at least one drawing/photograph executed in color. Copies of such color drawing(s) will be provided by the United States Patent & Trademark Office upon request and payment of the required fee.

FIG. 1 CY1 and CY2 are closely related Group 2/Class 2 ULVs. A. Genome organization of ULVs. Group 2/Class 1 members only encode two replication-required proteins and have extensive 3′ UTRs. Group 2/Class 2 members that infect dicots (except CY1) have an additional ORF (ORF5) that overlaps with the end of the RdRp ORF in the −1 frame. Monocot-infecting Class 2 members have an addition embedded ORF of different lengths. Group 2/Class 3 members have at least one additional ORF that are unrelated to each other and ORF5. B. Maximum likelihood phylogenetic tree based on RdRp nucleotide sequences. Branch numbers indicate bootstrap support in percentage out of 1000 replicates. The scale bar denotes nucleotide substitutions per site. The tree is mid-point rooted. Dicot-infecting and monocot-infecting Class 2 ULVs separate into different clades with the exception of parsley umbra-like virus (PULV), which is closer to an ancestral viral molecule that possibly gave rise to all Class 2 ULVs. C. Left, schematic secondary structure of CY1 and CY2. CY2 shares a similar overall secondary structure and contains two large insertions (shaded). Approximate location of start codons and stop codons are indicated by arrows.

FIG. 2 illustrates schematically a comparison of the CY1 RNA structure with structures for other Class 2 ULVs. Designations of CY1 structures (Pr, H5, H4a and H4b) are highly conserved and denoted for each genome structure. Inserted segments not found in CY1 are shown in dark grey. Open circle, closed circle and star denote ORF1 initiation site, ORF1 termination site and ORF2 termination site, respectively. Open triangle and closed triangle denote start site and termination site for ORF5, respectively.

FIG. 3 illustrates schematically a comparison of the CYVaV (CY1) and CYVaV-Delta (CY2) structures.

FIG. 4 shows modifications to an unstable insert that produced a stable insert

FIG. 5 illustrates stabilization of siRNA targeting GFP in CY1 genome. Panels A, B and C show the complete hairpin structures targeting GFP mRNA in Nicotiana benthamiana isolate 16C, which expresses GFP. Panel A: 21 nt GFPsh (sh: small hairpin) with the stabilizing loop sequence (ucaagag) from the pSuper vector (Brummelkamp T R, et al, 2002 DOI: 10.1126/science. 1068999). The minimum free energy (AG) of the structure is-39.30 kcal/mol. The polynucleotide sequence shown in Panel A is tgaagcggcacgact tcttcaatcaagagatgaagaagtcgtgccgcttca (SEQ ID NO:68). Panel B: 26 nt GFPsh with the same loop sequence as in A (AG is-51.20 kcal/mole. The polynucleotide sequence shown in Panel B is tgaagcggcacgacttcttcaagagcatcaagagagctctt gaagaagtcgtgccgcttca (SEQ ID NO:69). Panel C: 30 nt GFPsh. The loop sequence is gaauuc and ΔG is-63.80 kcal/mol. Finding PCR bands smaller than expected indicates that these hairpin structures are not stable after systemic infection. Furthermore, the relative band intensity of the small band (vector with insert deleted) and the bigger band (vector with intact insert) in the PCR results suggests that structural stability of the hairpin is reversely correlated with its maintenance in the vector after systemic infection. The polynucleotide sequences shown in Panel C are tgaagcggcacgacttcttca agagcgccaga (SEQ ID NO:70) and tctggcgctcttgaagaagtcgtgccgcttca (SEQ ID NO:71). Panel D: The secondary structure of CY1 established by SHAPE data (left) and the reference structure to be mimicked, CY2220-2280 (in dashed gray box, right). The ΔG of this structure is-31.50 kcal/mol. The polynucleotide sequence shown in Panel D is ggttagggtaactcacataccttcttccataactggaaaaggtcgtgtgagcaacctaac c (SEQ ID NO:72). Panel E: GFPmmck59. Total 59 nt mimicked structure includes 28 nt siRNA of sense orientation (circled in gray). GFPmmck59 (tgaagcggcacgacttctt caagagcgataactcgccttgacagaagtccaacgcttca (SEQ ID NO:73)) was inserted at position 2304 in CY1 (arrow), was stable after systemic infection, and was functional in silencing GFP (gray in picture indicates tissue where GFP is silenced). The RT-PCR result and the sequencing chromatogram proved the exceptional stability of the insert after systemic infection. * The minimum free energy was calculated using RNAfold Webserver: http://rna.tbi.univie.ac.at//cgi-bin/RNAWebSuite/RNAfold.cgi*. The polynucleotide sequence shown in Panel E is:

(SEQ ID NO: 8)
aaaaggtcgtgtgagcaacctaaccagttaatgtaggtgtctttccgtat
gaagcggcacgacttcttcaagagcgataactcgccttgacagaagtcca
acgcttcatctagtcaggatggtaagcaacccgtttatctgtacggcgct
cacccgtgggtaggaag.

FIG. 6 demonstrates the stability of two exemplary mimic inserts in the CY1 genome. Panel A: Locations of the inserts in one of the two-insert constructs. The natural hairpin at 2219 is deleted (i.e. bases 2220-2280 are deleted) and one of the two mimic inserts (targeting Callose Synthase 7) is inserted at that location (i.e between wild type 2219 and 2281). The second mimic insert (targeting CTV) is at 2304 (wild type numbering). The sequence of Mmck6.1 is SEQ ID NO:9. The sequence of CS7-V2.2 is SEQ ID NO:10. Panel B: In vitro translation of this construct (gray asterisks) in wheat germ extracts compared with WT CY1. Panel C: Sequencing the population of the two-hairpin construct at 4 weeks postinfiltration.

The polynucleotide sequence of F3-C3 #3 is:

(SEQ ID NO: 11)
actagaggaagtgttgacgaaatgtaatgttccagttatggaatcattaa
tttcgtccatcacttcctacggaaagacacctacattaactggtatcatt
tgcagacggaagattggcagttatgccaaatcttcgtctgcccattgata
ccaac.

The polynucleotide sequence shown in Panel D is:

(SEQ ID NO: 12)
ttggttagggtaactcacataccttcttccatatgaagcggcacgactat
ctagtcannntggtaagcannncgtttatctgt.

No variants were detectable. Many more combinations of two-insert hairpins were made and all were stable. As shown in Panel C, RT-PCR should indicate a single amplified product, with batch sequencing showing little to no heterogeneity. For comparison, sequencing of an unstable construct is shown in Panel D.

FIG. 7 illustrates wild-type (WT) CY1 (alternatively called CYVaV) and its nearest relative, CY2 (alternatively called CYVaV-Delta), from cannabis. CY2 has two inserts and other changes that allows it to encode an additional ORF.

FIG. 8 illustrates the stability of a sequence copied from a region of opuntia umbra-like virus (OULV) (OULV 2349-2491, 143nt, ΔG is-55.60 kcal/mol) and inserted into the CY1 genome. Referring to Panel A, left image, the secondary structure of OULV RNA genome is shown; the enlarged chimeric sequence (marked with light-brown circle) and its secondary structure is in the dashed box below. Referring to Panel A, middle image, the secondary structure of CY1 is shown; the enlarged structure of the insertion site in CY1 genome is in the dashed box below and the insertion point is marked with a gray arrow. Referring to Panel A, right image, the secondary structure of CY23300ULV (CY1 with OULV 2349-291 inserted between 2330/2331 of CY1) is shown; the enlarged secondary structure of CY1 2330 location with chimeric OULV insert is in the dashed box is shown. RT-PCR results using the cDNA of the three infected plants by CY2330OULV are shown in Panel B. Samples are labeled as CY2330OULV-1, CY2330OULV-2, and CY2330OULV-3, respectively, with sequencing identification presented in parentheses. PCR was performed using a primer set targeting the region from 1996 to 2452 of CY1 genome. pET17CY2330OULV and pCB301CY2330OULV were used for positive control. Sequence alignment results of the three samples are shown in Panel C. The presented sequence of CY1_wt is SEQ ID NO:1 (2276-2372). The presented sequences of CBC460, CBC461 and CBC462 are identical (SEQ ID NO:13):

taaccagttaatgtaggtgtctttccgtatctagtcacgatggtaagcaa
cccgtgcgtgttacgtcagtgacaatcgccatggaaccggcaatgtctac
ctctacccaacactgtggggtaggactcgctactagcggggcctactctt
caggtgagggttttggtaacttgttcaagtacaagcgcgcttgtaacttt
atctgtacggcgctcacccgtgggtaggaaggtgaaggtt

The presented sequence of the OULV-insert is (SEQ ID NO:14):

tgcgtgttacgtcagtgacaatcgccatggaaccggcaatgtctacctct
acccaacactgtggggtaggactcgctactagcggggcctactcttcagg
tgagggttttggtaacttgttcaagtacaagcgcgcttgtaact

Chromatogram images of the three samples above are shown in Panel D. The overall sequencing quality was very stable, except the thymine (T) at position 117 of the insert. The chromatogram signal showed weak noise of adenine (A), which may indicate that a small population of CY2330OULV may have A instead of T at position 117.

FIG. 9 illustrates the stability of duplicated internal structure (1132-1329) of CY1 at 2220/2280 location in CY1 genome at 8 weeks after systemic infection. Referring to Panel A, left image, the schematic image for the construction strategy is shown; the original hairpin-like structure between the location 2220 and 2280 was removed and the long-hairpin-like structure spanning from nucleotide positions 1132 to 1329 was cloned at the deleted region. Referring to Panel A, right image, the overall structure of CY2220_(1132-1329)_2280 is shown, which is the outcome of the left image. Referring to Panel B, RT-PCR using systemically infected tissues by CY2220_(1132-1329)_2280 is shown. In the gel image, the size of PCR bands from infected tissues were identical to the positive control (plasmid construct). PCR was performed using a primer set targeting the region from positions 1996 to 2452. Referring to Panel C, sequencing results are shown of PCR products and their alignment to the original sequence, CYVaV 1132-1329. The presented sequence of the duplicated-insert (1132-1329) corresponds to SEQ ID NO: 1 (1132-1329). The presented sequence of CY2220 (1132-1329) 2280-1 and CY2220 (1132-1329) 2280-2 are identical (SEQ ID NO:15):

attgctcgagcttcgttgcaguaaaagguuguauggugagggugcugagc
cguguaucgccaaaggccuaaaugcauuagaaucuggagcgacuuugagg
cgcaaaugggagaaguuuuuucuccaguuugcguuucucucgaccuucca
gguucgaccugcauguaagcguuggcaugcuaaaguucacacacaagcua
uaugacuauuacugcagttaatgtaggtgtctttccgt

Referring to Panel D, for more detailed information about the stability of the internally duplicated CYVaV construct, the PCR products from infected tissue were cloned into pMiniT2.0 cloning vector and a total of 44 clones were sequenced to get higher resolution for virus population after long-term infection. The sequencing information revealed that most of the population of the virus was very stable. The internally copied-insert of 29 clones out of 44 samples was identical to the original sequence. 13 out of 44 clones only had single nucleotide changes. The single base change written in black parentheses indicates that the base change happened in a single clone. Among the 13 clones, 2 clones had the same base change at nucleotide location 119 [(T119C)X2]. Multiple base changes in individual clones were marked as the same color. 1 out of 44 clones had two base changes and was marked by orange color, C49T, T111C. 1 out of 44 clones had three base changes and was marked by green color, T111C, T121C and T120C. Except for T113C and T187C, most of the base changes happened near the boundary between loop and stem. There was no major deletion or substitution from all of the samples. The polynucleotide sequence presented in Panel D (SEQ ID NO:16):

cagtaaaaggttgtatggtgagggtgctgagccgtgtatcgccaaaggcc
taaatgcattagaatctggagcgactttgaggcgcaaatgggagaagttt
tcttctccagtttgcgtttctctcgacgcttccaggttcgacctgcatgt
aagcgttggcatgctaaagttcacacacaagctatatgactattactg

FIG. 10 shows sequence alignment results of samples of FIG. 9 shown in Panels A, B, C, D and E thereof. The presented sequence of the Duplicated-insert (1132-1329) corresponds to SEQ ID NO:1 (1132-1329). The presented sequences of CBC135, CBC135, CBC137, CBC138, CBC142, CBC143, CBC144, CDE966, CDE968, CDE969, CDE970, CDE972, CDE975, CDE979, CDF018, CDF019, CDF022, CDF023, CDF024, CDF025, CDF026, CDF030, CDF031, CDF032, CDF038, CDF039, CDF040, CDF043, CDF044, and CDF045 are identical (SEQ ID NO:17):

ggtcattggtttaccgatgatacctgttcagaataggattgctcgagctt
cgttgcagtaaaaggttgtatggtgagggtgctgagccgtgtatcgccaa
aggcctaaatgcattagaatctggagcgactttgaggcgcaaatgggaga
agttttcttctccagtttgcgtttctctcgacgcttccaggttcgacctg
catgtaagcgttggcatgctaaagttcacacacaagctatatgactatta
ctgcagttaatgtaggtgtctttccgtatctagtcacgatggtaagcaac

The presented sequence of CBC139 (G22T) is (SEQ ID NO:18):

ggtcattggtttaccgatgatacctgttcagaataggattgctcgagct
tcgttgcagtaaaaggttgtatggtgatggtgctgagccgtgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CBC140 (C33T) is (SEQ ID NO:19):

ggtcattggtttaccgatgatacctgttcagaataggattgctcgagct
tcgttgcagtaaaaggttgtatggtgagggtgctgagctgtgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDE967 (T119C) is (SEQ ID NO:20):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgttcctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDE974 (G62C) is (SEQ ID NO:21):

ggtcattggtttaccgatgatacctgttcagaataggattgctcgagct
tcgttgcagtaaaaggttgtatggtgagggtgctgagccgtgtatcgcc
aaaggcctaaatgcattacaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDE977 (T111C, T121C, T170C) is (SEQ ID NO:22):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagcttgcgtttccctcgacgcttccaggttcga
cctgcatgtaagcgttggcatgctaaagctcacacacaagctatatgac
tattactgcagttaatgtaggtgtctttccgtatctagtcacgatggta
agcaac

The presented sequence of CDE978 (T35C) is (SEQ ID NO:23):

ggtcattggtttaccgatgatacctgttcagaataggattgctcgagct
tcgttgcagtaaaaggttgtatggtgagggtgctgagccgcgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDE980 (C49T, T111C) is (SEQ ID NO:24):

ggtcattggtttaccgatgatacctgttcagaataggattgctcgagct
tcgttgcagtaaaaggttgtatggtgagggtgctgagccgtgtatcgcc
aaaggtctaaatgcattagaatctggagcgactttgaggcgcaaatggg
agaagttttcttctccagcttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDF015 (C174T) is (SEQ ID NO:25):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcatacacaagctatatgac
tattactgcagttaatgtaggtgtctttccgtatctagtcacgatggta
agcaac

The presented sequence of CDF016 (G125A) is (SEQ ID NO:26):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgtttctctcaacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDF017 (C57T) is (SEQ ID NO:27):

ggtcattggtttaccgatgatacctgttcagaataggattgctcgagct
tcgttgcagtaaaaggttgtatggtgagggtgctgagccgtgtatcgcc
aaaggcctaaatgtattagaatctggagcgactttgaggcgcaaatggg
agaagttttcttctccagtttgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDF020 (T148C) is (SEQ ID NO:28):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgtttctctcgacgcttccaggttcga
cctgcacgtaagcgttggcatgctaaagttcacacacaagctatatgac
tattactgcagttaatgtaggtgtctttccgtatctagtcacgatggta
agcaac

The presented sequence of CDF037 (T119C) is (SEQ ID NO:29):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgttcctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDF041 (T187C) is (SEQ ID NO:30):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagtttgcgtttctctcgacgcttccaggttcga
cctgcatgtaagcgttggcatgctaaagttcacacacaagctatacgac
tattactgcagttaatgtaggtgtctttccgtatctagtcacgatggta
agcaac

The presented sequence of CDF047 (T113C) is (SEQ ID NO:31):

ggtcattggtttaccgatgatacctgttcagaataggattgctcgagct
tcgttgcagtaaaaggttgtatggtgagggtgctgagccgtgtatcgcc
aaaggcctaaatgcattagaatctggagcgactttgaggcgcaaatggg
agaagttttcttctccagttcgcgtttctctcgacgcttccaggttcga
cctgcatgtaagcgttggcatgctaaagttcacacacaagctatatgac
tattactgcagttaatgtaggtgtctttccgtatctagtcacgatggta
agcaac

The presented sequence of CDF049 (T101C) is (SEQ ID NO:32):

ggtcattggtttaccgatgatacctgttcagaataggattgctcgagc
ttcgttgcagtaaaaggttgtatggtgagggtgctgagccgtgtatcgc
caaaggcctaaatgcattagaatctggagcgactttgaggcgcaaatgg
gagaagtttccttctccagtttgcgtttctctcgacgcttccaggttcg
acctgcatgtaagcgttggcatgctaaagttcacacacaagctatatga
ctattactgcagttaatgtaggtgtctttccgtatctagtcacgatggt
aagcaac

FIG. 11A shows wild-type hairpin-like structures of CY1 ranging in size from 33 to 198 nt (labeled 1 to 4). (FIG. 49?) and shows the sequence and secondary structure of a hairpin-like insert mmck6.2 (SEQ ID NO:33).

FIG. 11B shows RT-PCR analysis and Sanger sequencing chromatographs from Mexican lime leaves infected with recombinant CYVaV1 having mmck6.2 after 12 months.

FIG. 11C shows: A. Northern blot of RNA extracted from two infected citrus plants after about one year of infection with recombinant CY1 including mmck6.2. B. Reverse-transcriptase (RT)-PCR is used to amplify a segment of the infecting VIGS vector. For this plant, 10 leaves were assayed. Only a single product of the correct size was found after 15 months of infection. C. Total RNA from young leaves of the infected plant was subjected to batch sequencing of the insert region. Top box is from 15-month citrus showing no indication of single “double peaks” that would denote base changes or multiple double peaks that would indicate a portion of the population contain deletions. In addition, at least 10 clones were generated for individual sequencing and no changes from the initial VIGS vector were found. D. Photo of infected Mexican lime plant. E. Nucleotide sequence of insert:

(SEQ ID NO: 34)
gtttcagtatttcgtcatcaccttcctaacggaagtgatggacgaaatta
atgaaacc.

FIG. 12 shows: A. Flow chart for how the data in B was generated. Note that color coding shown on the inserts (from RNAFold) are relative entropy values for the specific insert and go from low entropy to high entropy. Positional entropy values for each nucleotide, obtained from the folding program, go from 0 (white) to 2.6 (dark gray) and are absolute values that allow for comparisons between hairpins. Positional entropy values for individual residues are then rearranged for better visualization of the entire insert (far right). B. APE values for various hairpin-like structures (and some additional non-hairpin-like structures) inserted into CY1. Sorted positional entropy is shown for each insert (dark gray to lightest gray). The first four hairpin-like structures (boxed) were natural duplicates of hairpin-like structures 1 through 4. Other hairpin-like structures and non-hairpin-like inserts are arranged in order of descending APE (from 1.30 to 0.07). Mimic hairpin-like structures begin with a number (1 or 4) that identifies the hairpin-like structure being mimicked. Thick lines beneath each lane denote the length of each hairpin (number of nucleotides shown at right). Asterisks denote inserts that were not stable in the VIGS vector. Double asterisk denotes a hairpin-like structure that had three consecutive G:C pairs at the base and five consecutive G:C pairs in the apical region contributing to the very low APE. Deletion of two nucleotides on the 5′ side and two nucleotide changes (G to C) on the 3′ side of the hairpin-like structure in the apical region that elminiated the 5 consecutive G-C pairs generated a stable hairpin-like structure with no consecutive apical G:C pairs and the APE value changed to 0.15. Thick black arrow denotes an unstable hairpin-like insert with a relatively low APE value of 0.39 that contained a large number of higher entropy nucleotides.

FIG. 13 shows some elements of RNA secondary structure.

FIG. 14 shows sequences and secondary structure of A-wild type CY1 hairpin-like structure 4 of FIG. 49 (SEQ ID NO:35) and exogenous hairpin-like structures B—mmck15 (SEQ ID NO:36); C—mmck8 (SEQ ID NO:37); D—mmckpsvD (SEQ ID NO: 38); and E—mmckpsvE (SEQ ID NO:39).

FIG. 15 shows sequences and secondary structure of A-wild type CY1 hairpin-like structure 4 of FIG. 49 (SEQ ID NO:35) and exogenous hairpin-like structures B—PRSVmmck1 (SEQ ID NO:40) and C—PSRVmmck2 (SEQ ID NO:41).

FIG. 16 shows sequences and secondary structure of A-wild type CY1 hairpin-like structure 1 of FIG. 49 (SEQ ID NO:42) and exogenous hairpin-like structures B—PDS-mmck-1 (SEQ ID NO:43) and C—PDS-mmck-2 (SEQ ID NO:44).

FIG. 17 shows sequences and secondary structure of A-wild type CY1 hairpin-like structure 1 of FIG. 49 (SEQ ID NO:42) and exogenous hairpin-like structures B—LcrGyr-mmck5 (SEQ ID NO:45) and C—LcrGyr-mmck3 (SEQ ID NO:46).

FIG. 18 shows sequences and secondary structure of A-wild type CY1 hairpin-like structure 1 of FIG. 49 (SEQ ID NO:42) and exogenous hairpin-like structures B—Clas-GyrAmmck-5′ (SEQ ID NO:47) and C—Clas-GyrAmmck-3′ (SEQ ID NO:48).

FIG. 19 shows sequences and secondary structure of A-wild type CY1 hairpin-like structure 1 of FIG. 49 (SEQ ID NO:42) and exogenous hairpin-like structures B—CS 7 (SEQ ID NO:49).

FIG. 20 shows sequences and secondary structure of A-wild type CY1 hairpin-like structure 1 of FIG. 49 (SEQ ID NO:42) and exogenous hairpin-like structures B—Erwinia GyrA (SEQ ID NO:50).

FIG. 21 shows SHAPE changes resulting from insertion of CY2301GFP30sh into CY1. The presented polynucleotide sequence is SEQ ID NO:51.

FIG. 22 shows the sequence and secondary structure of M2250GFP30ext, an unstable insert targeting GFP. The presented polynucleotide sequence is (SEQ ID NO: 52):

gguuaggguaacucacugaagcggcacgacuucuucaagagcgccauuca
gugagcaaccuaacc.

FIG. 23 shows the sequence and secondary structure of GFPmmck59, a stable insert targeting GFP. The presented polynucleotide sequence is (SEQ ID NO:53):

tgaagcggcacgacttcttcaagagcgataactcgccttgacagaagtcc
aacgcttca.

FIG. 24 shows the sequence and secondary structure of GFPmmck63, an unstable insert targeting GFP. The presented polynucleotide sequence is (SEQ ID NO:54): tgaagcggcacgacttcttcaagagcgccataatcggcgccttgacagaagtccaacgct tca.

FIG. 25 shows the sequence and secondary structure of CTV-insert-natural-V2.2, a naturally occurring hairpin-like structure in CTV. The presented polynucleotide sequence is (SEQ ID NO:55):

ggggguuuauguuuggcaaagaaaguguuggaacuguuagucaaggggug
guugaaucguuucuc.

FIGS. 26A and 26B show RT-PCR and Sanger data for recombinant CYVaV1 with insert-natural-V2.2 infecting N. benthamiana after 3 weeks. The presented polynucleotide sequence is (SEQ ID NO:56):

tcgttgggggtttatgtttgcaaagaaagtgttggaactgttagtcaagc
gggtggttgaatcgtttctcgtttgaagcggaaaaccgctcgtttaacgt
ccttcgctaatttgttgcttgcgaggctctcagttaat

FIG. 27 shows an unstable insert BBLv2-1 (SEQ ID NO.57 and SEQ ID NO:58).

FIG. 28 shows the addition of a heterologous insert including a cleavage site and a peptide into the RdRp of CY1.

FIG. 29 shows siRNA generated from CTV targeting inserts quantifed by stem-loop RT-PCR from leaves collected from locations in multiple plants.

FIG. 30 shows siRNA generated from CTV-targeting inserts quantifed by stem-loop RT-PCR from young and mature leaves collected from citrus plants.

FIG. 31 shows a wild type hairpin-like structure of CY1 and hairpin-like structures targeted against DMR6 genes.

FIG. 32 shows a wild type hairpin-like structure of CY1 and hairpin-like structures targeted against MLO genes

FIG. 33, for poliovirus and CVB3, a wild type region and two inserts were designed, one stabilized according to the methods described herein which includes adjustment of the hairpin stability and in these examples surrounding sequence modification, and one control construct with straightforward fully base-paired hairpin insertion in the viral genome.

FIG. 34 shows, for the control poliovirus construct, sequence heterogeneity observed in passage 0.

FIG. 35 shows, for the stabilized insert in the poliovirus genome, sequence heterogenicity observed in passages 0 and 10.

FIG. 36 shows, for the control insert in the CVB3 genome, sequence heterogenicity observed in passages 0, 5 and 10 and for the stabilized insert in the CVB3 genome, sequence heterogenicity observed in passages 0 and 10.

FIG. 37 shows the secondary structure of the CVB3 insert and the location of the deletions and insertions.

FIG. 38 shows the secondary structure of the poliovirus insert and the location of the deletions and insertions.

FIG. 39 shows the secondary structure of full-length CY1 wherein numbers 1 to 4 denote natural CY1 hairpin-like structures.

### DETAILED DESCRIPTION OF EMBODIMENTS

### Definitions

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a virus” includes a single virus as well as a plurality or population of the virus. Generally, nomenclatures utilized in connection with, and techniques of molecular biology, protein and oligonucleotide or polynucleotide chemistry and hybridization described herein are those well-known and commonly used in the art.

The term “or” is generally employed in its sense including “and/or” unless the content clearly dictates otherwise.

The recitation of numerical ranges by endpoints herein includes all numbers and fractions subsumed within that range (e.g., 1 to 5 includes 1, 1.5, 2, 2.75, 3, 3.90, 4, and 5).

As used herein, the term “targeting” and variations thereof, refers to a sequence that inhibits a pathogen or modifies a biological function in a host. In some embodiments, a targeting sequence specifically binds, or produces in the host a molecule that specifically binds, a target sequence, molecule or cell. “Specifically binding” refers to having greater affinity to a particular molecule or cell of interest relative to alternative sequences, molecules or cell types. In some embodiments, a targeting sequence encodes a peptide, for example an antimicrobial peptide. In some embodiments, a targeting sequence produces a bioactive RNA molecule, for examples an siRNA.

The terms “mutation” or “modification” as used herein refers to insertions, substitutions or deletions of one or more nucleotides within a sequence.

The term “sequence identity” as used herein, unless stated otherwise, refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity= [number of identical overlapping positions]/[total number of positions]×100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The term “effective amount” as used herein refers to any amount of a compound or a composition that is sufficient to generate a desired response, including but not limited to preventing or reducing the chance of disease onset, slowing disease progression, and alleviating symptoms.

The term “wildtype” or “native” as used herein refers to a reference genotype, including a genotype that predominates in a natural population or a laboratory population as compared to a natural or a laboratory mutant form, and also includes an isolate or consensus sequence.

The term “functional variant” as used herein includes modifications of the polypeptide sequences disclosed herein that perform substantially the same function as the polypeptide molecules disclosed herein in substantially the same way. For example, a nucleotide sequence encoding the functional variant may comprise sequences having at least 50%, at least 60%, at least 70%, at least 80%, or at least 90%, or at least 95% sequence identity to the sequences disclosed herein. The functional variant may also comprise conservatively substituted amino acid sequences of the sequences disclosed herein.

The terms “heterologous segment” or “heterologous RNA segment” as used herein refer to a nucleotide sequence that is not naturally present in a particular reference molecule, for example the genome of a wild type virus, from which a vector or other RNA molecule containing the heterologous segment may be derived. The heterologous segment may contain an active portion which may be derived from, or may be complementary to, or a sequence present in another organism, for example a host of the virus or a pathogen of the host. The heterologous segment may also contain a portion that completes a hairpin-like structure with the active portion. The heterologous segment may provide any coding or non-coding function appropriate for a particular application. The terms “heterologous” and “exogenous” have been used herein interchangeably.

The term “homolog” as used herein refers to the same sequence in a related species such as the same sequence in a different virus. Typically homologs share a high degree of sequence identity, such as at least 50%, 60%, 70% or more. The homology between two sequences that are derived from species which are more closely related is typically higher than from more distantly related species.

As used herein, the terms “vector” or “RNA vector” refer to an RNA molecule containing a heterologous segment with an active portion that performs a desired function in a host. The desired function may involve, for example, regulation of the host (e.g. host gene downregulation), inhibiting a pathogen of the host (optionally including in the case of plants and insect feeding on the plant), or modifying replication of the vector in the host (i.e. attenuating replication of the vector itself as in a live attenuated vaccine). The RNA vector may be, for example, a viral vector or a sub-viral vector. In some examples, the RNA vector is a plus-sense single stranded RNA, wherein the term single stranded RNA may include folded RNA with double stranded or base-paired regions. The RNA vector may be derived from a virus or from sub-viral RNA. For example, an RNA vector may be derived from a plant virus such as CY1, CY2, another ULV, CTV or, TRV.

As used herein, a “host” refers to a cell, tissue or organism capable of being infected by and capable of replicating a nucleic acid. A host may include a whole plant, a plant organ, plant tissue, a plant protoplast, and a plant cell. A plant organ refers to a distinct and visibly differentiated part of a plant, such as root, stem, leaf, seed, graft or scion. Plant tissue refers to any tissue of a plant in whole or in part. Protoplast refers to an isolated cell without cell walls, having the potency for regeneration into cell culture, tissue or whole plant. Plant cell refers to the structural and physiological unit of plants, consisting of a protoplast and the cell wall. Alternatively, a “host” may include an animal, such as a human, or an organ or cell thereof.

As used herein, “nucleic acid sequence,” “polynucleotide,” “nucleotide” and “oligonucleotide” are used interchangeably and refer to a polymeric form of nucleotides of any length. Polynucleotides may have any three-dimensional structure, and may perform any function. A “gene” refers to a polynucleotide containing at least one open reading frame that is capable of encoding a particular polypeptide sequence. “Expression” refers to the process by which a polynucleotide is transcribed into mRNA and/or the process by which the transcribed mRNA is translated into peptides, polypeptides, or proteins.

RNA sequences presented in this specification or the figures may use either “t” or “u” to denote uracil. Sequences may be presented in upper case or lower case letters. There is no significance attached to the case (upper case or lower case) of the sequence unless noted in the description of the sequence.

A vector “derived from” a particular molecule, for example the genomic RNA of a virus, means that the vector contains genetic elements or sequence portions from such molecule. In some embodiments, the vector comprises a replicase open reading frame (ORF) from such molecule. The resulting vector may be capable of replicating in plant or animal cells. A vector may be constructed from the molecule from which it is derived. One or more heterologous segment(s) may be added as an additional sequence to the vector. The backbone of a vector (i.e. excluding any heterologous segments) may have 60% or more, 70% or more, 75% or more, 80% or more, 85% or more, 90% or more, 95% or more or 98% or more sequence identity with the RNA molecule (e.g. the genoimic RNA of a wild type virus) from which is is derived.

As used herein, the terms “infection” or “capable of infecting,” with respect to a vector of the present invention, includes the ability of such vector to transfer or introduce its nucleic acid into a host, such that the nucleic acid or portion(s) thereof is replicated and/or proteins or other agents are synthesized or delivered in the host. Infection also includes the ability of a selected nucleic acid sequence to integrate into a genome of a target host.

As used herein, the term “endogenous” refers to a polypeptide, nucleic acid or gene that is expressed by a host.

As used herein, the term “hairpin” refers to a primarily base paired segment of RNA that comprises a fully base-paired region (alternatively called a “stack”) and a non-base paired region (alternatively called “an apical loop”) between the bases on opposite sides of the stack.

As used herein, the term “hairpin-like structure” refers to a primarily base paired segment of RNA having multiple stacks separated by non-base-paired regions, alternatively called “bulges” or “internal loops” (sometimes called “loops” for brevity when the distinction from an apical loop is apparent from the context), and an apical loop between the bases on opposite sides of the stacks. A loop may be symmetric, with an equal number of bases on opposite sides of the loop, or asymmetric, with a different number of bases on opposite sides of the loop. The term “bulge” may be used as an alternative name for a loop, or may be used in some contexts to refer specifically to an asymmetric loop having one or more non-paired bases on only one side. Most hairpin-like structures are linear (i.e. without junctions) but in some embodiments a hairpin-like structure is Y-shaped, T-shaped or otherwise branched. The linear branches of a branched structure may also be hairpin-like structures themselves.

As used herein, the term “junction” refers to a non-base-paired region that separates three or more stacks. A simple junction separating three stacks may create, for example, a Y-shaped or T-shaped secondary structure. A stack connected to a junction may be part of a hairpin, a hairpin-like structure, or a stem.

As used herein, the term “stem” refers to a structure that extends from a junction that does not terminate in an apical loop. A stem may connect a junction to another junction, or may connect a junction to the single stranded regions between stems (i.e., single stranded regions that are not apical loops, internal loops or junctions). Referring to FIG. 53, the junction separates a stem below the junction from two hairpin-like structures above the junction.

As used herein, the term “movement protein” refers to a protein(s) required for cell-to-cell and/or long distance movement in a plant. “Coat protein” refers to protein(s) comprising or building the virus coat.

As used herein, “virus” includes traditional or conventional viruses and related infectious agents that might or might not satisfy all elements of some definitions of a virus, for example because they do not encode a coat protein and/or a movement protein. For example, plant viruses include ULV, for example CY1. Virus also includes derivatives, sub viruses, variants or parts of a virus, for example defective RNA, subgenomic RNA and RNA multimers (e.g. dimers or trimers), that may be infectious in a host alone or in the presence of the full virus or a helper virus.

The terms “about”, “substantially”, “essentially”, “approximately” and similar terms as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies. Further, unless stated otherwise or indicated by the context, elements not explicitly modified by the terms “about”, “substantially” and “approximately” include a similar reasonable amount of deviation. In the context of sequence identity, sequence described using the terms “about”, “substantially”, “essentially”, “approximately” and similar terms, or not explicitly modified by these terms, include sequences having 70% or more, 80% or more, 90% or more, or 95% or more sequence identity with a reference sequences, unless stated otherwise or implied otherwise, for example because a larger degree of identity is required to provide a stated function or structure.

As used herein, the term “secondary structure” describes the folding of single stranded RNA into base-paired and non-base-paired regions. Hairpins, hairpin-like structures and junctions are elements of the secondary structure of RNA molecules. An RNA molecule may also have a tertiary structure. The tertiary structure includes interactions, i.e. base pairing, of segments of the RNA separated in a manner other than by way of junctions or apical loops. In some cases the tertiary structure includes long-distance interactions between RNA segments separated by many bases and/or intermediate hairpins or hairpin-like structures.

The term “RNA bioactive molecule” or “bioactive RNA” refers to any biologically active RNA molecule. In an embodiment, the RNA bioactive molecule is an RNAi effector molecule for inducing an RNA interference response. In an embodiment, the RNAi effector molecule is short interfering RNA (siRNA), microRNA (miRNA), long hairpin (lhRNA), short hairpin (shRNA), double stranded (dsRNA), or anti-sense RNA. In an embodiment, the RNAi effector molecule is dsRNA. In another embodiment, the RNAi effector molecule is lhRNA. The terms “RNA interference,” “interfering RNA” or “RNAi” refer to single-stranded RNA or double-stranded RNA (dsRNA) that is capable of reducing or inhibiting expression of a target nucleic acid.

The term “antisense RNA” refers to a single stranded RNA that is complementary to messenger RNA and that hybridizes with the messenger RNA blocking translation into protein.

The term “long hairpin RNA” or “IhRNA” as used herein refers to a long inhibitor RNA that can be used to reduce or inhibit expression of a target nucleic acid by RNA interference. LhRNA are typically single stranded with secondary structure (hairpin) and longer than 60 nucleotides. Total length may be 1000 base pairs or more.

The term “siRNA” or “siRNA oligonucleotide” refers to a short inhibitory RNA that can be used to reduce or inhibit nucleic acid expression of a specific nucleic acid by RNA interference. The siRNA can be, for example, a duplex, a short RNA hairpin (shRNA) or a microRNA (miRNA).

### Vectors and Heterologous Segments

This specification describes heterologous segments (alternatively called inserts) provided in RNA vectors that are stable in replication, for example more stable than fully base paired hairpins carrying the same active portion. The vectors are.typically stable in replication (i.e. progeny of the vector carry the heterologous insert with minimal mutation) for 3 weeks or more but many embodiments are stable for longer periods of time, for example 3 months or more. The inserts are described as “hairpin-like” to indicate that they include base-paired segments but are not fully base-paired beyond an apical loop.

According, provided herein is a ribonucleic acid (RNA) vector comprising a segment derived from a virus and a heterologous segment(s) inserted therein, wherein the heterologous segment comprises a) two or more base-paired regions, b) one or more non-base-paired regions separating the base-paired regions, and c) an apical loop at the end of one of the based paired regions. The heterologous segment is designed according to one or more parameters or rules described herein.

In some embodiments, a hairpin-like region of an RNA vector includes a hirpin-like heterologous segment appended to a truncated native hairpin-like segment. In these cases, the parameters or rules described herein may be applied to the appended heterologous segment alone, or to the entire resulting hairpin-like segment combining the heterologous segment and the truncated native segment.

In some embodiments, the parameters or rules are defined with reference to one or more hairpin-like regions in a wild type RNA virus from which the RNA vector is derived. However, in other embodiments, the parameters or rules are determined without reference to the wild type RNA virus from which the RNA vector is derived.

The inserts are sometimes referred to as “mimics”. One or more aspects of the inserts were developed initially through studies of some naturally occurring hairpin-like segments in some umbravirus-like viruses (ULVs). However, these studies were later expanded to include investigations or other viruses and to synthetic hairpin-like segments. Accordingly, the term “mimic” is now used loosely and no longer implies that there is any similar naturally occurring hairpin-like structure.

Because of the path of development indicated above, and because many studies of ULVs are recent, this specification includes an extensive discussion of ULV. However, the invention is not limited to ULV unless expressly stated.

Plant RNA viruses typically encode multiple proteins including one or more subunits of the RdRp, one or more movement proteins, a coat protein and a silencing suppressor. Plant RNA virus proteins can also have more than a single function.

One notable exception is the umbraviruses. Umbraviruses do not encode a coat protein or a silencing suppressor. However, umbraviruses are always found in nature in association with a specific helper virus from the luteovirus, polerovirus or enamovirus genera. The umbravirus uses the coat protein of the helper virus to produce a capsid/virion. The umbravirus also benefits from the silencing suppressor of the helper virus. However, the umbravirus produces its own movement proteins, one of which the helper virus uses to extend its own ability to colonize a plant.

Another notable exception are umbravirus-like viruses (ULV). ULV are plus-sense viral or sub-viral RNAs that infect plants. ULV have some similarities with umbraviruses, for example an RdRp that is generated by −1 ribosomal frameshifting. ULV may also have an umbravirus-related 3′ terminal structure. However, ULV do not encode umbravirus-related movement proteins. At least some ULV do not encode any movement proteins. ULV are not umbraviruses or otherwise in the family Tombusviridae. However, since the taxonomic analysis of at least some ULV is recent, some ULV (in particular some Group 1 ULV or Group 2/Class 1 ULVs as defined further below) may have been previously identified as umbraviruses.

Although some ULV have been described previously as subviral RNA, or may be subviral RNA according to some definitions for example because they do not form virions or encode coat proteins or other proteins characteristic of most plant viruses, the word “virus” as used herein will include sub viral RNA unless stated otherwise. The term “subviral RNA” may be used, for example, to refer to truncated progeny of ULVs such as defective RNA (D-RNA) and subgenomic (sgRNA).

Based on phylogenic analysis, ULVs can be divided into two groups. Group 1 is a catch-all grouping currently containing: AgULV or ageratum virus 1 (e.g. GenBank OP660856); ArULV (alternatively called AULV) or Arborcitae umbravirus-like virus (e.g. GenBank OQ102001); GULV-2 and GULV-3 or Grapevine umbravirus-like virus (e.g. Gen Bank OR947507); and GuiULV (alternatively called GPpTV1) or Guiyang Paspalum umbravirus-like virus (e.g. GenBank OM514399). Group 2 ULV include, among others, the viruses (or sub-viral RNA) that were previously referred to as umbravirus-like associated RNA (ulaRNA) in Structural Analysis and Whole Genome Mapping of a New Type of Plant Virus Subviral RNA: Umbravirus-Like Associated RNAs, Liu J. et al., Viruses 2021, 13, 646. Group 1 ULV are more closely related to umbraviruses than Group 2 ULV. Group 1 ULVs have lengths from 3.3-4.3 kb. AgULV has been found in plants with a helper virus. AULV has been found in plants without a helper virus.

Group 2 ULVs, are viral or subviral RNAs with monopartite, plus-strand genomes of about 2.7 to 4.5 kb. Group 2 ULVs code for at least two proteins which are both required for replication. One protein is a 5′ proximal product (expressed from open reading frame 1, ORF1). The other protein is the RdRp, which is generated by −1PRF extension of the ORF1 protein. Many Group 2 ULVs, including WULV, PUV, Bab VQ, CY1, CY2, FULV, OULV, PULV, MULV and JgULV have been found in natural hosts in the absence of a helper virus. All dicot-infecting Class 2/Group2 ULVs known to date have been found in plants in the absence of a helper virus.

Group 2 ULVs can be separated into 3 classes. Class 1 do not encode any additional proteins beyond their replication required proteins and have an extensive 3′ UTR. Class 3 ULVs have one or two additional ORFs at least one of which overlaps the RdRp ORF. Class 2 members that infect dicot hosts contain one additional ORF (optionally called ORF5) that also partially overlaps the RdRp ORF. Class 2 members that infect monocot hosts have a third ORF (optionally called ORF5 and is the same protein as found in the dicot-infecting Class 2 members) overlapping the RdRp and also most have an additional ORF (optionally called ORF6) embedded within ORF5. One exception to the description above is citrus yellow vein associated virus 1 (CY1). CY1 is a dicot Class 2 ULV that does not have ORF5 due to two large deletions and other mutations. CoULV, found in Taro, is an exception among monocot-infecting Class 2 ULV that does not contain ORF6.

Group 2 ULV (and some Group 1 ULV) are further distinct from umbraviruses because umbraviruses have an intergenic region (typically 100-300 nt) between their replication required ORFs and any additional ORFs. Group 2 ULV either have no ORFs beyond their replication required ORFs, or have a third ORF adjacent the RdRP ORF. Some Group 2 ULV also have a fourth ORF. There are no material intergenic region between the ORFs of most ULV, AgULV being an exception.

Class 1 ULV, examples of isolates in GenBank, and their host plants include: a) PMeV2 (KT921785)-papaya; b) PUV (MT113180)-papaya; and c) BabVQ (MT113182)-babaco. It is noted that some Class 1 ULVs have been previously described as umbraviruses, which is currently known to be incorrect.

Class 2 dicot infecting ULVs, examples of isolates in GenBank, and their host plants include: a) citrus yellow vein associated virus CY1 (JX101610, MZ330089, MZ330113)-citrus; b) citrus yellow vein associated virus 2 CY2 (MT893741, MT893740)-hemp; c) fig umbra-like virus FULV (MW480892, MW480893)-fig; d) opuntia umbra-like virus OULV (MH579715, MT909563, MT909562)-cactus; e) parsley umbra-like virus PULV (OM419177)-parsley.

Class 2 monocot infecting ULVs, and examples of isolates in GenBank, include: a) Teosinte umbra-like virus TULV (OK018180)-teosinte; b) Johnson grass umbra-like virus JgULV (OM937760)-Johnsongrass; c) Ethiopian maize-associated virus EMaV2 (MF415880) and EMAV1 (MN715238)-corn; e) sugarcane umbra-like virus SULV (MN868593)-sugarcane; f) maize umbra-like virus MULV (OM937759)-corn.

Class 3 ULVs, and examples of isolates in GenBank, include: a) SbaVA (MK211274)-strawberry; b) WULV (OK573479)-wheat; c) GULV (OP88631)-grapevine; and, d) PIULV (OL330774)-Poaceae liege.

The GenBank sequences at the identifiers given above are incorporated by reference. Unless specified otherwise, the reference sequence of CY1 is GenBank JX101610, which is SEQ ID NO:1 herein.

Some ULVs are described in detail in Structural Analysis and Whole Genome Mapping of a New Type of Plant Virus Subviral RNA: Umbravirus-Like Associated RNAs, Liu J. et al., Viruses 2021, 13, 646. It was noted in this publication that some ULVs express a protein with movement protein-like motifs. Without intending to be limited by theory, the inventors currently believe that the proteins described in that paper are not movement proteins but may instead be coat proteins (it is known in the art that movement proteins of the 30K class and coat proteins can be hard to distinguish and are related to each other). Accordingly, they will typically be called coat proteins herein since further experimentation showed that they do not have conserved features and properties found in all 30K class movement proteins. However, the vectors described herein are not dependent on whether these are coat proteins, movement proteins or any other type of protein.

A description of some ULVs, an in particular Group 2/Class2 ULVs, is provided in-1 Programmed ribosomal frameshifting in Class 2 umbravirus-like RNAs uses multiple long-distance interactions to shift between active and inactive structures and destabilize the frameshift stimulating element, Mikkelsen A. et al., Nucleic Acids Res. 2023 Oct. 2; 51 (19): 10700-10718, which is incorporated by reference. In particular, this publication describes six tertiary interactions that are conserved in Class 2 ULVs and two tertiary interactions that are conserved in all Group 2 ULVs. Optionally, the presence of these structures and interactions when present in at least one comformation, optionally in combination with other indicia, may be used to identify ULVs. Some of the relevant structures and interactions can be summarized as: a) a Y-shaped folded region, in particular a Y-shaped region having a sequence and/or secondary structure similar to that of other ULVs, b) a hairpin-like region with a ribosome recoding area downstream of the Y-shaped region with an apical loop that interacts (by way of complementary bases) with a further downstream region, c) interaction (by way of complementary bases) of a hairpin like region upstream of the Y-shaped region with a hairpin like region downstream of the Y-shaped region.

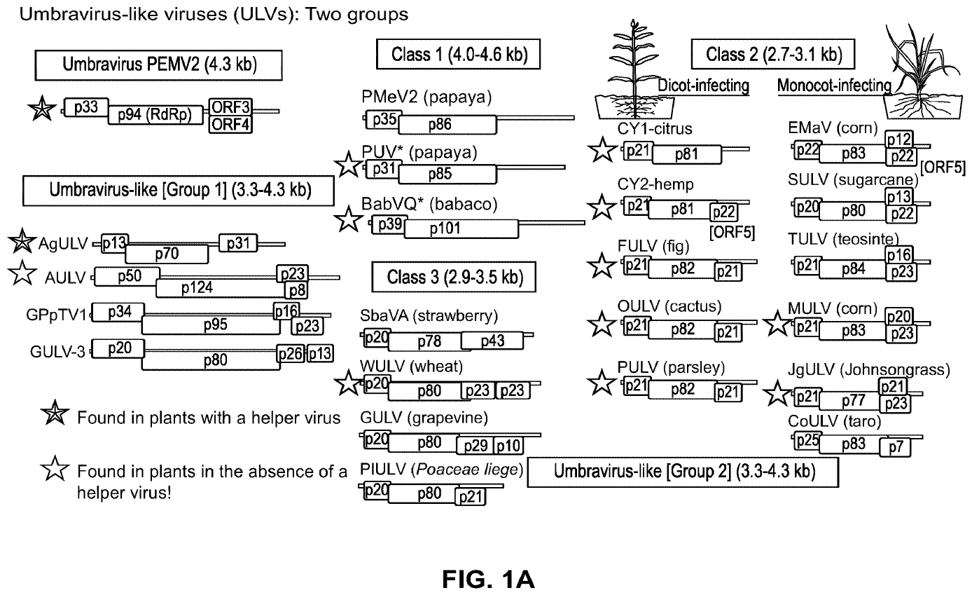

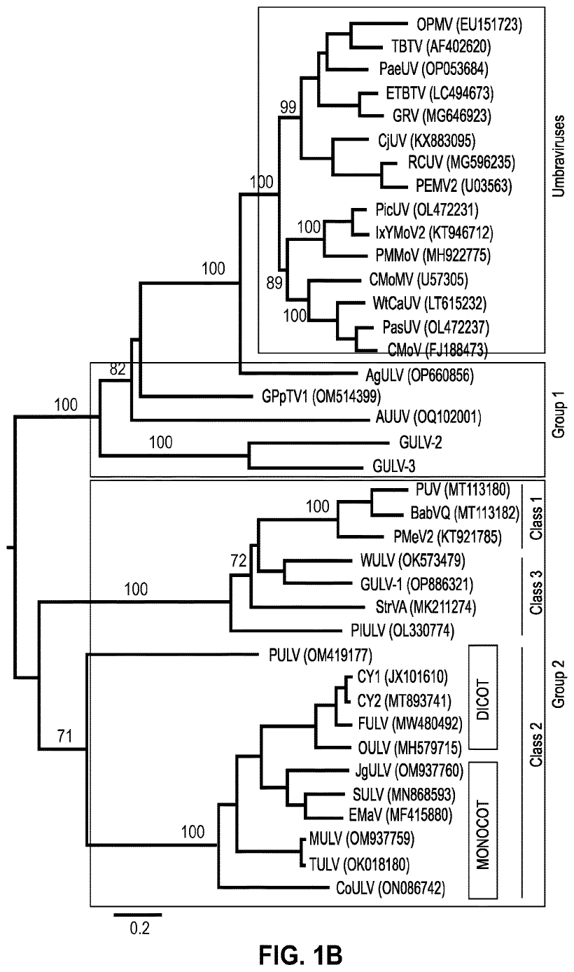

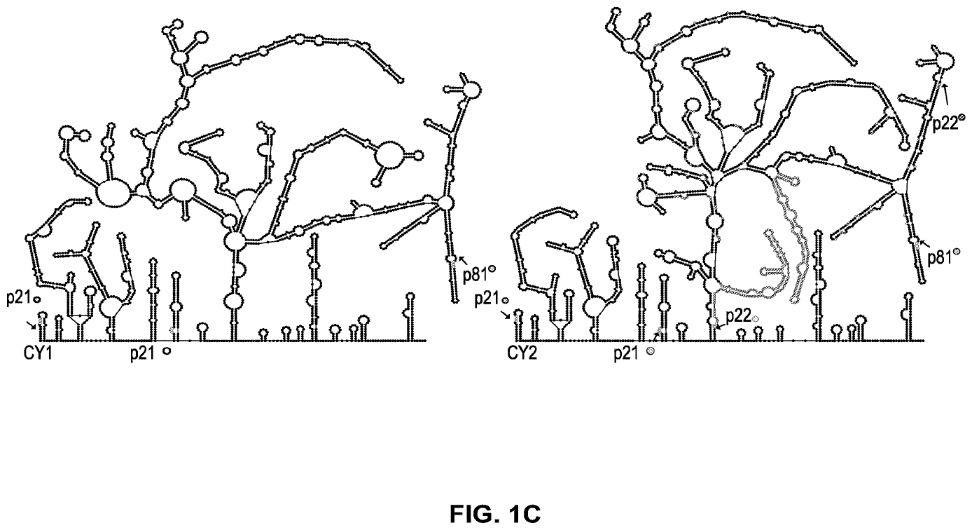

FIG. 1 Panel A shows the genome organization of some Group 2 ULVs. As indicated therein, some ULVs have only replication required ORFs (the first two ORFs). Class 2 ULVs have one or two additional ORFs without intergenic regions between the additional ORFs and the replication-required ORFs or between multiple non-replication required ORFs. Class 3 ULVs have one or two non-replication required ORFs without intergenic regions between the additional ORFs and the replication-required ORFs or between multiple non-replication required ORFs.

FIG. 1, Panel B shows a maximum-likelihood phylogenetic tree used to evaluate the relationships of ULVs to all known umbraviruses based on RdRp nucleotide sequences. Alignments were generated and trimmed in MEGA X [92]. General time reversible model with gamma distribution rates and invariable frequency (GTR+G+I) was determined to be the best-fit substitution model in MEGA X and was implemented to construct the tree. The following ULV sequences were used: PMeV2 (KT921785), PUV (MT113180), BabVQ (MT113182) [42, 93, 94], OULV (MH579715, MT909563, MT909562) [44], FULV (MW480492, MW480492) [60], CY1 (JX101610) [40], CY2 (MT893741) [77], WULV (OK573479) [95], GULV (OP886321) [96], PIULV (OL330774) [97], SbaVa (MK211274) [98], TULV (OK018180) [99], MULV (OM937759) [41], JgULV (OM937760) [41], SULV (MN868593) [100], EMaV (MF415880), AgULV (OP660856), GuiULV (OM514399), ArULV (OQ102001 [101]. To analyze the phylogenetic relationship of ORF5 proteins with selected CPs and MPs, protein sequences were aligned through PROMALS3D and trimmed in MEGA X. The LG model with gamma distribution rates and invariant frequency was determined to be the best-fit substitution model in MEGA X and was implemented to construct the tree.

FIG. 1, Panel C shows the secondary structure of CY1 and CY2. CY2 is the closest presently known relative of CY1, and might optionally be considered a strain of CY1. CY2 differs from CY1 in having a subgenomic promoter and two long segments not found in CY1. However, CY2 has over 85% or over 90% sequence identify with CY2 as measured by clustal W score (about 90.2% for CY2: MT893741 and CY1: JX101610). Other known dicot infecting Class 2 ULVs have over 70% sequence identity with CY1 except for PULV (found in parsley) which has over 54% sequence identity with CY1 (about 54.1%). Other known monocot infecting Class 2 ULVs also have over 54% sequence identity with CY1. In comparison, the umbravirus PEMV2, which is the closest known relative to CY1 that is not a ULV, has a sequence identity of only about 50% (50.3%) with CY1. Some Class 1 and Class 3 ULVs or Group 1 ULV may also have sequence identities with CY1 of less than 54%, or less than 50%, but are considered to be a ULV and not an umbravirus for example because they do not encode movement proteins similar to umbravirus movement proteins, because they do not have ORFs other than replication required ORFs, because they lack an intergenic region between replication required ORFS and any other ORFs, and/or because they have secondary structures or tertiary interactions similar to CY1 or other Class 2 ULVs. As many ULVs have not been extensively studied, their naming and classification may change over time. Some ULVs that are separately named herein may, in future, be considered to be strains of a single virus. In some examples, the backbone of a vector may have at least 54%, at least 70%, at least 85% or at least 90% identity with SEQ ID NO: 1.

ULVs infect and replicate in plants. Although ULVs do not have umbravirus-like movement proteins, and at least some have no movement proteins at all, they are able to move within the phloem of plants. In at least some ULV, this movement is believed to result from specific binding of a portion of the RNA genome with phloem protein 2 (PP2), a common protein made by many plants. The portion of the RNA genome responsible for PP2 binding is believed to be upstream of ORF2.

At least some ULV also do not encode a silencing suppressor. Without intending to be limited by theory, ULV may be able to survive without silencing suppressors by having a very high rate of replication. In at least some examples, ULVs may make about six times more polymerase for replication than other viruses. Given the high rate of replication, even through some of the progeny are cut by the DICER-like enzymes of the host plant, some of the progeny remain as full length sequences for further replication. When used as a vector, the high rate of replication combined with the action of the host plant DICER-like enzymes causes the production of non-coding exogenous inserts (for example siRNA) at high rate. In at least some examples, coding exogenous inserts may also be produced at a high rate.

ULVs are capable of phloem limited movement in plants despite, in at least some examples, not having movement proteins. CY1 clearly does not have a movement protein and yet moves through the phloem of many plants. In the case of Class 2 dicot infecting ULVs, as demonstrated by examples using CY2 and OULV, when the third ORF (ORF5) is disabled, the ULVs are still able to move thorough the phloem of the plant. Without intending to be limited by theory, movement through the plant may be enabled by binding of the ULV with phloem protein 2 (PP2), a common protein produced by many plants.

Defective RNA (D-RNA) of CY1 lacks nucleotides 672 to 2419. However, this D—RNA also binds to PP2. D—RNA of CY2, also contains portions of the 5′ and 3′ ends and is predicted to also bind to PP2.

The present disclosure also describes the use of ULVs other than CY1, for example other Class 2 dicot ULVs, as vectors. In some embodiments, CY2 is used as a vector. In other embodiments, a ULV infecting opuntia (OULV) is used as a vector. However, given the high degree of similarity between ULVs, and in particular between Class 2 dicot ULVs, other ULVs including other Class 2 dicot ULVs may be similarly converted into vectors and are expected to bind to PP2 and move within the phloem of plants.

PP2 binds to all RNA to some extent. References to binding to PP2 herein refer to specific binding, or to binding that is more efficient than the binding between PP2 and random RNA sequences.

CY1 does not produce virions whereas as CY2 does produce virus-like particles. However, CY1 produces agglomerates with PP2 in host plant sap. Since CY2 in the absence of ORF5 also moves systemically with similar timing, tropism, and symptoms as CY1, it likely also binds to PP2 in plants and has been shown to bind to PP2 in vitro using Northwestern assays. In addition to providing movement, binding to PP2 may improve the response of the host plant to diseases, such as citrus greening, wherein harm to the plant may be related to excess PP2 production and/or PP2 sieve plugging in response to the pathogen.

Without intending to be limited by theory, current hypotheses regarding ORF5, the movement of ULVs, and the evolution of some plant viruses include the following:
1. ULVs, with a few exceptions, encode their own RNA polymerase and capsid-like protein.
2. Group 2 ULVs, and particularly Class 2 ULVs, generate copious amounts of polymerase by −1PRF; silencing suppressors may not be needed.
3. Unlike umbraviruses, ULVs are with few exceptions found in the absence of a helper virus, possibly because they do not need one as some of them encode their own capsid protein.
4. ORF5 produces a capsid-like protein related to the P3 coat protein of luteoviruses, poleroviruses, enamoviruses, and sobemoviruses (the helper viruses for umbraviruses).
5. CY1 has lost the ability to encode the capsid-like protein and might no longer be transmitted to new plants since transmission vectors (e.g., insects like aphids) require virions.
6. With few exceptions, ULVs use host movement proteins for movement.
7. For CY1, CY2 and other Class 2 ULVs, the host movement protein is likely PP2, which is the same movement protein used by the hop stunt viroid.
8. Ancient plant viruses may have systemically infected plants without encoded movement proteins.
9. Movement proteins may be the progenitors for some coat proteins.

It is currently unknown if or how CY1 may have been transmitted from plant to plant in nature. Since CY1 has only been found 4 times in nature, all in same grove of trees at the same time, it is possible that CY1 is a fragment of another virus that has not been located. However, CY1 and other ULV are capable of replication and movement when introduced into a plant, for example by grafting, transfer through dodder or agroinfiltration (agroinfection).

CY1 was originally found in limequat trees and readily infects citrus trees and some other host plants. Some previous publications suggest that CVEV is a helper virus for CY1, but CY1 is no longer considered to have, or at least not to require, a helper virus.

CY1 contains two open reading frames, ORF1 and ORF2, both of which are required for replication. The ORF2 protein is generated as a fusion protein with the ORF1 protein after a ribosome-1 frameshift.

CY1 replication in plants naturally produces two major long non-coding fragments, F281 having nucleotides 1-281 and F671 having nucleotides 1-671. Without intending to be limited by theory, F671 may be RdRp-derived and F281 may be exonuclease-derived. Other fragments may also be produced. Defective RNA (D-RNA) are also produced having, for example, nucleotides 1-671 and 2420-2692. The D—RNA is replicated by the CY1 polymerase and moves in host plants. Surprisingly, the D—RNA can also replicate in the absence of CY1 and can move systemically at low levels independent of CY1. Roots may act as a repository of full-length RNA while D—RNA are a significant fraction in the whole plant.

The polynucleotide sequence (bases 1 to 2692) of the CY1 isolate at GenBank JX101610 is presented below (SEQ ID NO:1):

| | |
|---|---|
| ggguaaauau ggauccuuca ucuuugcccc gugccuguug gcaucaugcc | 50 |
| agacaggugu uucgagcauc aacuagcuuc ucaagagagg ugguucgcgc | 100 |
| ugcucguaga uggguuacca ugcccaccag ucgccaugca uaugacuuuu | 150 |
| caacgagucu aggcauugug auugcugagc cugcagcucg uuuacgacgc | 200 |
| cgucugcccu cuguacgaaa gugcgcagag aaguuaguag uccacaagca | 250 |
| agucgacacu uugguggacg aauggugcuc uggaauuccc aacccugaua | 300 |
| ucguagaagu ugguugggca cuccgucuga gggaccguuu cggucuuccu | 350 |
| cccgcuucug agccuacccg gcucaguggu gagagauggg ugcucaaaca | 400 |
| acucaauggg guagauccug agucauggaa ugcugaucuu gguaggucag | 450 |
| uucauaucca aggagacuac gccccaggga ggaaugccca uaucgcucag | 500 |
| gucgcggcga ccuugugguu aacuaggacc uugcaugaca aggccuuggc | 550 |
| ucgccaccag gguuuucgcg auuugcagug auuggggucg acgggcuaga | 600 |
| ggcaaaagca gugccucuag cuucuggacu ccgacugcuu ccgguuccgc | 650 |
| gacccggaca aagucgacga cugucucaga ccuuguuacu uccaacaccu | 700 |
| cgugcucaau ucgugaauca cgcgugcucg gcuaacaacc uuggacgugu | 750 |
| gaugaccaca cguguguugc aguacaaggg ccgagauccg auccuucccu | 800 |
| cuucugaagc ccuucaccga cuuaaccuuc ggauagcuga gcuauauagg | 850 |
| ucuagaccuu cuaccgucua uccauuaagu uaugaagggu uucucaauug | 900 |
| cuaugaaggc cgacagcgua cucguuacgc ccaagccguc gagcaguuga | 950 |
| ugcgguccac ucuugagccg aaagaugcgc gaguugaaac guucauuaag | 1000 |
| aacgagaaau uugacugggc guugaaaggg gaggaggcug auccucgagc | 1050 |
| aauccaacca aggaagccga aauauuuggc ugagguugga cggugguuca | 1100 |
| aaccuuugga gcgaaucauc uacaaggauc ucaguaaaag guuguauggu | 1150 |
| gagggugcug agccguguau cgccaaaggc cuaaaugcau uagaaucugg | 1200 |
| agcgacuuug aggcgcaaau gggagaaguu uucuucucca guuugcguuu | 1250 |
| cucucgacgc uuccagguuc gaccugcaug uaagcguugg caugcuaaag | 1300 |
| uucacacaca agcuauauga cuauuacugu aagucuccca cucuccagcg | 1350 |
| cuaucucaaa uggacacucc gcaaccaugg cgucgccucc ugcaaagaau | 1400 |
| ugucauauga guaugagguu guuggccgga gaaugagugg ugacauggac | 1450 |
| acugcauugg gcaacugcgu cauuaugucg auacuuacau gguuuaugcu | 1500 |
| uagugaacuu ggcauuaagc augaauuauu cgauaauggu gacgauuguu | 1550 |
| uguucauuug cgagucucac gacgucccca gccccgaggu aauuacaaac | 1600 |
| ugguuuucgg acuuuggguu ugugguuagg uuggaaggcg ucacguccgu | 1650 |
| guuugagcgu auugaguuuu gccaaacuuc cccaguaugg acugagaggg | 1700 |
| guuggcugau guguaggaau auuaagucau ugaguaaaga ccuuacgaau | 1750 |
| guuaauucgu gcacgggcuc cacgauugaa uauacccacu gguugaaagc | 1800 |
| agugggaaag ugcgggucaa uacucaaugc ugguguaccu auauuucagu | 1850 |
| ccuuucacaa caugcuggaa aggcuuggca cuaacucucg uauugaucga | 1900 |
| gggguuuucu ucaaaucagg gcuaguuaau cucauucgug ggauggacag | 1950 |
| gcagccugac guugacauca cuacuuccgc ucggcuuucu uucgaagugg | 2000 |
| cauucgggau aacacccggg augcaauugg cuauugaacg guacuaugac | 2050 |
| ucugucaugg gcucgcugag uaaaauagaa acaacuaagu ggccaauuga | 2100 |
| acuaagaaag gaauacgaac acggaaguga gugguacgag gacuuaggcg | 2150 |
| uccuaggaug aauaggguca uugguuuacc gaugauaccu guucagaaua | 2200 |
| ggauugcucg agcuucguug guuaggguaa cucacauacc uucuuccaua | 2250 |
| acuggaaaag gucgugugag caaccuaacc aguuaaugua ggugucuuuc | 2300 |
| cguaucuagu cacgauggua agcaacccgu uuaucuguac ggcgcucacc | 2350 |
| cguggguagg aaggugaagg uuuugugucc uuuaggucuu ggacagucug | 2400 |
| cgggcuuggg aacgacgccc cgcuagcaac guacugcucu ccuaccggac | 2450 |
| ugguagcuua auugucaucu uggagcgaua gcacuguggg ccucacccuu | 2500 |
| cgcgcguugg acguguugcg ugccccccac agauuuguga aacucuaugg | 2550 |
| agcaguuccg cgagccagaa gggaggaugg ccgccuggcg uaauccagga | 2600 |
| gcucuggggg gcuuguacuc agaguagcau ucugcuuuag acuguuaacu | 2650 |
| uuaugaacca cgcgugucac guggggagag uuaacagcgc cc | 2692 |

The 3′ end of CY1 includes bases 2468 to 2692. The 3′ Cap Independent Translation Enhancer (3′ CITE) of CY1 includes bases 2468-2551. ORF1 which encodes p21 includes bases 9 to 578. ORF2 which encodes p81 includes bases 752 to 2158.

The amino acid sequence of protein p21 is presented below (SEQ ID NO:2):

MDPSSLPRACWHHARQVFRASTSFSREVVRAARRWVTMPTSRHAYDESTS
LGIVIAEPAARLRRRLPSVRKCAEKLVVHKQVDTLVDEWCSGIPNPDIVE
VGWALRLRDREGLPPASEPTRLSGERWVLKQLNGVDPESWNADLGRSVHI
QGDYAPGRNAHIAQVAATLWLTRTLHDKALARHQGERDLQ

The amino acid sequence of protein p81 is presented below (SEQ ID NO:3):

MTTRVLQYKGRDPILPSSEALHRLNLRIAELYRSRPSTVYPLSYEGFLNC
YEGRQRTRYAQAVEQLMRSTLEPKDARVETFIKNEKEDWALKGEEADPRA
IQPRKPKYLAEVGRWEKPLERIIYKDLSKRLYGEGAEPCIAKGLNALESG
ATLRRKWEKFSSPVCVSLDASREDLHVSVGMLKFTHKLYDYYCKSPTLQR
YLKWTLRNHGVASCKELSYEYEVVGRRMSGDMDTALGNCVIMSILTWEML
SELGIKHELEDNGDDCLFICESHDVPSPEVITNWFSDFGFVVRLEGVTSV
FERIEFCQTSPVWTERGWLMCRNIKSLSKDLTNVNSCTGSTIEYTHWLKA
VGKCGSILNAGVPIFQSFHNMLERLGTNSRIDRGVFFKSGLVNLIRGMDR
QPDVDITTSARLSFEVAFGITPGMQLAIERYYDSVMGSLSKIETTKWPIE
LRKEYEHGSEWYEDLGVLG

The polynucleotide sequences of recoding frameshift sites of CY1 is presented below:

(SEQ ID NO: 4)
ucgcucaggucgcggcgaccuugugguuaacuaggaccuugcaugacaag
gccuuggcucgccaccaggguuuucgcgauuugcagugauuggggucgac
gggcuagaggcaaaagcagugccucuagcuucuggacuccgacugcuucc
gguuccgcgacccgga
(SEQ ID NO: 5)
caaagucgacgacugucucagaccu
(SEQ ID NO: 6)
aggucuuggacagucugcgggcuugggaacgacg

CY2 was originally found in hemp but also infects some other host plants. CY2 is related to CY1 but differs from CY1 in having an additional open reading frame (FIG. 19A), a subgenomic promoter, two long segments not found in CY1, and other changes from CY1 that keep the ORF open (FIG. 19B). The additional open reading frame is optionally called “ORF5” (FIG. 19A), the designations of ORF3 and ORF4 having been reserved for movement proteins of umbravirues in some previous publications comparing CY1 to umbraviruses. As discussed herein, this protein encoded by the additional open reading frame is currently believed to be a coat protein, although it might alternatively or additionally have another activity.

CY2, when infecting as host plant, produces a D—RNA, and sgRNA and at least one major non-coding fragments. Roots may act as a repository of full length RNA while D—RNA are a significant fraction in the whole plant.

CY2 (and other ULV) contains two open reading frames, ORF1 and ORF2, similar to those of CY1, both of which are required for replication. The ORF2 protein is only generated as a fusion protein with the ORF1 protein after a ribosome-1 frameshift. Expression of the ORF5 protein in CY2 (and possible other ULVs) is most likely from the sgRNA.

The sequence of the CY2 used in experimental examples is a modification (believed to be a correction) of the sequence in GenBank MT893741 and is presented below (SEQ ID NO: 7):

GGGTAAATATGGAATCTTCATCTTTGCCCCGTGCCTGCTGGCAAAAGGCC
AGGCAGGTGTTGCGAGCACCAGCTACCTTC
TCGAGAGAGGTGGTCAAAGCTGCCCGCAAATGGGTCACCATGCCCAGTAA
CCACCGTGTCTACGACTACTCCACTAGTCT
GGGCATTGTGATTGCTGAGCCTGTGGCTCGTCTGCGACGCAGTCTACCCT
CCGTACGAAAGTGCGCAGAGAAGATAGTAG
TCCACCAGGGAATCGACACCTTGGTGGACGATTGGTGTACAGGACTTCCT
AACCCTGATATAGTGGAAGTTGGTTGGGCA
CTCCGTCTGAGGGACCGTTTCGGTCTTCCTCCCGCCTCTGAGCCTACTAG
GCTTAGTGGTGAGAGATGGGTGCTCAAACA
ACTCAATGGGGTAGATCCCGAGTCATGGAATGCTGATCTGGGTAAACCAG
TTCACGTCCAGGGAGACTACGCCCCAGGGA
GGAATGCCCACATCGCGCAGGTCGCGGCGACCTTGTGGCTAACTAAGACC
TTGTCTGACAAGGCCTTGGCCCGCCACCAG
GGTTTTCGCGATTTGCAGTGATTGGGGTCGACGGGCTAGAGGCTAAAGCA
GTGCCTCTAGCTGCAGGACTCCGACTGCTT
CCGGTTCCGCGGCCCGGACAAAGCCGACGGCTGTCTCAGACCTTGTTACT
ACCAACCCCTCGTGCACAATTTGTGAATCA
CGCGAACTCGGCTAATAACCTTGGGCGCGTGATGACCACACGAGTGATGC
AGTACAAAGGCCGAGACCCGATCCAACCCT
CTCAAGAAGCCCTAACCAAACTTAACCTTCGGATAGCCGAGCTATATAAG
TCGAGACCATCCACCGTCTATCCGTTGAGT
TATGAAGGGTTTCTCAACTGCTACGAAGGCCGACAGCGTACTCGTTACGC
TCAAGCCGTCGAGCAGCTGTTGAGGTCCAC
TCTAGAACCTAAAGATGCGAGAGTTGAAACGTTCATTAAGAACGAGAAGT
TTGATTGGGCGTTGAAAGGGGAGGAGGCTG
ATCCTCGTGCAATCCAACCAAGGAAGCCGAAATATCTGGCTGAGGTTGGA
CGGTGGTTTAAACCTTTGGAGCGAATCATC
TACAAGGATCTTAGTAAGCGGTTGTATGGCGAGGGTGGTGAACCTTGTAT
TGCCAAAGGCTTAAATGCACTAGAATCTGG
GGCGACTTTGAGGCGCAAATGGGAGAAGTTTTCTTCTCCTGTTTGCGTCT
CTCTTGACGCTTCCAGGTTCGACCTGCATG
TAAGTGTTGGCATGCTTAAGTTTACACACAAGCTGTACGACTACTACTGC
AAGTCTCCCACCCTCCAACGTTATCTCAAA
TGGACACTCCGCAATCACGGCACTGCCTCCTGTAAAGAATTGTCATATGG
TTATGAGGTAGAAGGCCGAAGAATGAGTGG
TGACATGGACACCGCATTGGGCAACTGCGTCATCATGTCGATACTAACAT
GGTTTATGCTTAGTGAGCTTGGCATTAAGC
ATGAATTATTCGACAATGGTGACGACTGCTTGTTCATTTGCGAGTCAAAA
GACGTCCCCAGCCCCGAGGTGATCACGAAC
TGGTTTTCGGACTTTGGGTTTGTGGTCAGGTTGGAAGGCGTCACGTCCGT
GTTTGAGCGTATAGAGTTTTGCCAAACTTC
CCCAGTATGGACTGAGAGGGGTTGGCTGATGTGTAGGAACATTAAGTCGT
TGAGTAAGGACCTTACGAATGTCAATTCGT
GCACGGGCTCCACAATTGAATATACTCACTGGTTAAAAGCCGTGGGGAAG
TGCGGGTCAATACTCAATGCTGGTGTGCCT
ATATTTCAGTCCTTTCACAACATGCTGGAAAGGCTTGGTACTAACTCTCG
TATAGATCGCGGGGTGTTCTTTAAATCTGG
GCTAGTCAATCTCATTCGTGGGATGGACAGACAACCTGACGTTAAGATCA
CCACTTCCGCTCGTCTTTCTTTCGAAGTGG
CATTCGGGATTACACCTGGAATGCAATTGGCTATTGAACGGTACTATGAC
TCAGTCATGGGCCCGCTGGGTAAAATAGAA
ACAACTAAATGGCCAATAGAACTAAGAAAGGAATACGAGTACGGAAGCGA
GTGGTACGAAGACTTAGGCGTCCTAGGATG
AGCAGGGACATCGGTTTACCGATGGCCCCCGTTCAGAATAGGATGGCTAG
GGCTTCGTTGGTTAGGGAAACCTATATACC
TTCTTCCACCACAACTGGTAAGGAGGCTGTGTGGGCAACCTGGCCAATTA
ATGTAGGTGTCTTTCCGCAACTAGCCACGA
TAGTAAGCAACCCGTTATACTGGCGTGTGACCAGTGTGCAAGTGGCAATG
GAGCCAGCAACGTCAACCTCTACCCAACTC
TGTGGTGTAGGTCTCAGTACATCTGGGGCTTACAACTCAGGGGAAGGGTT
CGGGAACACGTTTAAGCTCTTGCGCTCTTG
TAACTATACGAGACGCTCACCCGTGGGAGGGAACGTGATGGTCAAGTGGC
CCATCAGCATGCCCTACATTCTTAATGATG
ATGCGCACAAGACCACAGGTCTAACAGCTTGTGTGGTCATTGCTGTCACC
AATCCAGGTGCCATCACTGGCCAATCTTGG
GCGGAGATTCAATTGAATGTAGAGTACGTAGTGGGTACTTAGACAGTCTG
CGGGCTTGGGAACGACGCCCCGCTAGCAAC
GTACTGCTCTCCTACCGGACTGGTAGCCGAGCTGTTATCTTGGAGCGATA
GCACTGTGAGCCTCACCCTGCGCGCGATGG
ACGTGTTGCGTGACCCTCACAGATTTGTGAAACTCTATAGAGTAGTTCCG
CGAGCCAGAAGGGAGGATGGCGACCTGGCG
AAATCCAGGAGCTCTGGGGGGCTTGTACTCAGAGTAGCAATACTGCTTTA
GACTGTTAGTTTGATGAACCACGCGTGTCA
CGTGGGGAGAACTAACAGCGCCC

OULV was originally found in opuntia (a cactus) and a nearly identical version also infects hemp. OULV has over 70% sequence identity with CY1 and CY2.

The RNA vector described herein may comprise a backbone, typically derived from a wild type virus, and one or more heterologous segments inserted in the backbone. The backbone may include the genomic sequence of a virus, optionally with one or more modifications. The backbone may contain all or substantially all of the genomic RNA sequence of a wild type virus. The backbone may have 60% or more, 70% or more, 80% or more or 90% or more or 95% or more or 98% or more sequence identity with a wild type virus that the vector is derived from.

The backbone may be a wild type virus or a sequence derived from a wild type virus. Derivation implies the addition, removal or substitution of one or more nucleotides of the wild type virus. The wild type virus may be referenced by the sequence of an isolate of the virus or a consensus sequence. The derivation may include any modifications known in the art to produce suitable vectors.

The backbone typically maintains the ability to replicate in a host. In some ULV examples, the backbone retains the replication required ORFs, and optionally a PP2 binding site, of a reference virus in a functional form. However, the backbone may have modifications or deletions. In some embodiments, the deletions may include the removal of some or all of a hairpin-like region. In some embodiments, the modifications may include point mutations or short sequence modifications relating to the folding or thermodynamic stability of the vector. In some embodiments, the modifications may include attenuation or disabling of non-replication ORFs, which may be accomplished by methods known in the art.

Other modifications may include, for example, deletion or attenuation of portions of the sequence not required for replication and/or movement, deletion or attenuation of portions related to the safety of the vector such as production of symptoms in the host or transmission between hosts, or minor modifications for example to adjust the fitness of the virus or the stability of the secondary structure of the virus. In most cases, a sufficient portion of the wild type virus is retained such that the vector retains the native ability of the wild type virus to replicate in a host and, optionally, to also move within a host plant. The vector may also differ from the wild type virus in the way that isolates of a virus normally vary from other isolates or from a consensus sequence for a virus or subviral RNA. In some cases, the vector will have a sequence identify of at least 80%, at least 90% or at least 95% of the wild type virus or a dimer, trimer or other isomer thereof. The vector may also have multiple heterologous inserts, including inserts other than the inserts described herein.

Modifications to the backbone can include, for example, point mutations, small deletions or truncations, or removal or replacement of hairpin-like regions. Modifications to the backbone may include fitness or reverse fitness related modifications or deletion or truncation of hairpin like regions, either without replacement or with replacement with a heterologous hairpin like region, as described in International Publication Number WO 2023/215782, RNA Vectors with Hairpin-like Inserts, published on Nov. 9, 2023, describes.

In an optional aspect of reverse fitness, a hairpin-like or other structure of the wild type virus is removed in combination with adding one or more inserts. The insert or inserts may be added all in other locations, or an insert may be added in the former location of the structure that was removed. For example, a hairpin-like structure of the wild type virus may be removed and replaced with an heterologous hairpin-like structure. Optionally, the heterologous hairpin-like structure is similar in one or more aspects to the hairpin-like structure that was removed, for example in length, minimum free energy (alternatively called MFE or AG), average positional entropy, or secondary structure. Alternatively, the heterologous hairpin-like structure is unlike the hairpin-like structure that was removed, but is similar in one or more ways (e.g. length, minimum free energy (AG), average positional entropy, or secondary structure) to another hairpin-like structure in the wild type virus, or a relative of the wild type virus, or the heterologous hairpin-like structure is otherwise a hairpin-like structure as described herein.

The factors described herein may also be applied separately to a hairpin-like structure included as part of a Y-shaped, V-shape, or T-shaped insert or other insert with a junction.

Based on the observation of wild type hairpin-like structures and experiments involving heterologous hairpin-like structures, various additional parameters may be useful for insert stability: (1.1) the length limit of a fully base-paired region; (1.2) the length limit of the entire hairpin-like structures; (1.3) the length limit of consecutive G:C pairs; (1.4) the maximum number of non-paired bases in a symmetrical or asymmetrical internal loop; and, (1.5) the largest average positional entropy of a cluster (i.e. 10 per side) of base-paired nucleotides or an entire base-paired segment. For an RNA vector derived from a wild type virus, a heterologous segment may be used that does not exceed one or more of these parameters, or other parameters described herein, as determined by the wild type virus. In some examples, parameters derived from a wild type virus may be modified, for example by 50%, by 40%, by 30% or more.

In some embodiments, the heterologous segment further complies with one or more design guidelines or parameters. These guidelines or parameters for the heterologous segment may include: the average positional entropy (APE) is in the range of 0.01 to 0.75; the length of the heterologous segment is 300 nt or less; the maximum length of a base-paired region is 19 base pairs or 17 base pairs; the maximum APE of a base-paired region is 0.8; the maximum number of consecutive G:C pairs in a base-paired region is 4; the maximum number of bases in a non-base-paired region is 21 or 20; the ΔG of the exogenous segment is within a range of +20 to −20 kcal/mol, or −5 to +15, kcal/mol, or within 10 kcal/mol (+ or −) of the ΔG of a naturally 43ccurring hairpin of similar length; the standard deviation of PE is less than 0.5, less than 20% or less than 15% of bases have a PE greater than 1; the largest PE of any base is not greater than 2.0; and the insert, not considering the apical loop, is 65-90% base-paired. Although heterologous segments are optionally hairpin-like structures, a hairpin may be designed according to one or more of the design guidelines or parameters described herein.

Accordingly, in some embodiments, the heterologous segment has a minimum free energy similar to the corresponding structure of the wild-type RNA virus. In some embodiments, the heterologous segment has one or more parameters within (or within 50% of the limits of) a range of the parameter in one or more hairpin-like structures of a wild type virus, the one or more parameters selected from the group consisting of: average positional entropy (APE); secondary structure; maximum length of a base-paired region; maximum number of consecutive G:C base pairs; maximum number of A:U base pairs, maximum number of bases on one side or collectively on both sides in a non-base paired region; maximum APE of a base-paired region; minimum free energy relative to length; and maximum length of hairpin-like segment.

Designing hairpin-like structures that have the appropriate attributes, such as average positional entropy, may start with design of the active or targeting sequence on one side of the hairpin. This is matched by a partially complementary sequence on the other side that will produce the correct values, e.g. average positional entropy values and or ΔG relative to length, for the hairpin-like structure. The partially complementary sequence may have, for example 65-90% or 70-85% complementarity with the targeting sequence. Optionally, the partially complementary sequence may be designed to result in a hairpin-like structure similar in shape to a natural hairpin-like structure being mimicked and/or incorporate other parameters described herein.

In some embodiments, the heterologous segment has an average positional entropy (APE) less than 0.75, 0.65, 0.5, 0.4, 0.39, 0.36 or 0.32. In some embodiments, the APE is greater than 0.01, 0.02 0.03, 0.04, 0.05, 0.06 or 0.07. In some embodiments, the APE is from 0.01 to 0.75, 0.01 to 0.65, 0.01 to 0.5, 0.01 to 0.4, 0.01 to 0.39, 0.01 to 0.36 or 0.01 to 0.32. In some embodiments, the APE is from 0.02 to 0.75, 0.02 to 0.65, 0.02 to 0.5, 0.02 to 0.4, 0.02 to 0.39, 0.02 to 0.36 or 0.02 to 0.32. In some embodiments, the APE is from 0.03 to 0.75, 0.03 to 0.65, 0.03 to 0.5, 0.03 to 0.4, 0.03 to 0.39, 0.03 to 0.36 or 0.03 to 0.32. In some embodiments, the APE is from 0.04 to 0.75, 0.04 to 0.65, 0.04 to 0.5, 0.04 to 0.4, 0.04 to 0.39, 0.04 to 0.36 or 0.04 to 0.32. In some embodiments, the APE is from 0.05 to 0.75, 0.05 to 0.65, 0.05 to 0.5, 0.05 to 0.4, 0.05 to 0.39, 0.05 to 0.36 or 0.05 to 0.32. In some embodiments, the APE is from 0.06 to 0.75, 0.06 to 0.65, 0.06 to 0.5, 0.06 to 0.4, 0.06 to 0.39, 0.06 to 0.36 or 0.06 to 0.32. In some embodiments, the APE is from 0.07 to 0.75, 0.07 to 0.65, 0.07 to 0.5, 0.07 to 0.4, 0.07 to 0.39, 0.07 to 0.36 or 0.07 to 0.32.

A hairpin-like insert may have one or more of: no more than 19, no more than 17 or more no more than 13, or no more than 10, consecutive fully paired bases; no more than 300 nt, no more than 200 nt, or no more than 198 nt bases; no more than 4, or no more than 3, continuous G:C pairs; no more than 20 or no more than 15 bases in a loop (i.e. counting bases on both sides of a loop); an APE of no more than 0.8, or no more than 0.6, in any base-paired region. Optionally, a hairpin-like structure as described immediately above for insertion into a ULV may be inserted into a vector derived from another wild type virus.

In some embodiments, the heterologous segment mimics the secondary structure of the wild type hairpin-like structure by having a similar arrangement of base-paired regions and non-base-paired regions.

Regarding the maximum number of consecutive fully paired bases, the presence of A-U base pairs may allow for larger values. In CY1, the largest base-paired region (13 base pairs) had an A-U rich region. Stacks without A-U rich regions in naturally occurring hairpin-like structures in CY1 had no more than 10 consecutive paired bases.

In some embodiments, the base-paired regions of the heterologous segment have 19 or less base pairs or 17 or less base pairs or 13 or less base pairs or 10 or less base pairs.

In some embodiments, the base-paired region of the heterologous segment has no more than 4, or no more than 3 consecutive G:C base pairs.

In some embodiments, the heterologous segment has no more than 21 bases or no rmore than 20 bases or no more than 15 bases collectively on both sides in a non-base paired region (i.e. counting bases on both sides of a loop).

In some embodiments, the heterologous segment mimics the secondary structure of the wild-type hairpin-like structure by having a similar ΔG relative to length.

In some embodiments, a) each base-paired region of the heterologous segment has an APE of less than 0.8 or less than 0.6 and/or b) the ΔG of a region including two non-base paired regions and one base-paired region between them is not positive.

In some embodiments, the heterologous segment has a minimum free energy within 10 kcal/mol, or within 5 kcal/mol, or within 20 kcal/mol for an insert of at least 150 nucleotides, of a naturally occurring hairpin-like structure of similar size, i.e. of a size within 10% of the size of the heterologous segment, or a minimum free energy in a range of −5 to +15 of −0.44 multiplied by the length of the insert (# of nt)−1.89, or a minimum free energy in a range of −20 to +20 of −0.44 multiplied by the length of the insert (# of nt) −1.89 for an insert of 150 nucleotides or more.

In other embodiments, the heterologous segment has one or more of a) a minimum free energy within 10 kcal/mol, or within 5 kcal/mol of a wild-type hairpin-like structure of similar length or a line of best fit between multiple wild-type hairpin-like structures, b) a length within 4 bases, or within 2 bases, or the same as a hairpin-like structure of the wild type virus or the sub-viral RNA, and c) an arrangement of base-paired (paired) and non base-paired (looped) regions that is substantially the same as, or the same as, the hairpin-like structure of the wild type virus or the sub-viral RNA.

In some embodiments, the heterologous segment has between 40 and 300, 40 and 200, 60 and 300 or 60 and 200 bases.

The limit on the APE of the entire heterologous segment to be inserted tends to result in inserts having at most a small number of bases with high positional entropy. Alternatively or additionally, one or more other guidelines may be used, including: a) the insert does not have any bases with positional entropy greater than 2.0 or greater than 1.5, optionally with an exception that a large insert (e.g. an insert with more than 100 nucleotides) may have a small number (e.g. 1 or 2 or 3) of nucleotides with PE greater than 1.5, b) the insert does not have more than 15%, or does not have more than 10%, of bases with positional entropy greater than 1.0, and c) the standard deviation of of the postional entropy of the bases in an insert is 0.5 or less or 0.4 or less, for example in a range of 0.1 to 0.4.

In some embodiments, the heterologous segment has a standard deviation of PE less than 0.5 or less than 0.4. In some embodiments, a) less than 20% or less than 15% or less than 10% of the bases of the heterologous segment have a PE of greater than 1.0 or b) the largest PE of any base of the heterologous segment is not greater than 2.0 or not greater than 1.5, or both.

A mimicked heterologous segment may also follow one or more of the guidelines or parameters described above. Alternatively, a mimicked heterologous segment may follow one or more analogous guidelines or pararameters derived from a study of hairpin-like structures in a wild type virus that the vector is derived from, or one or more relatives of that wild type virus.

An insert is typically designed by designing an active or targeting sequence, which provides most or all of a first side of the insert (typically not including the apical loop as part of either side). A second side of the insert, on the opposite side of the apical loop, is designed to be mostly, but not entirely, complementary to the first side. For example, the second side may be 65-90% or 70-85% complementary with the first side. The lack of complete complementarity provides alternating base-paired regions and non-base-paired regions. Each of the base paired regions may have 19 or less, 17 or less, 13 or less or 10 or less base pairs. Each of the base paired regions should also not have more than 4 consecutive G-C base pairs, and optionally not more than 4 consecutive A-U base pairs. Direction of the base pairings is not considered, for example a G-C pairing followed by a C-G pairing is considered to be two consecutive G-C base pairings. Although other parameters described herein may be considered, in many examples hairpin-like structures are stable when designed considering only 65-90% or 70-85% complementary between the first side and the second side of the insert; each of the base paired regions having 19 or less, 17 or less, 13 or less or 10 or less base pairs; and each of the base paired regions have no more than 4 consecutive G-C pairs or no more than 3 consecutive G-C base pairs, and optionally no more than 4 consecutive A-U base pairs. If a hairpin-like structure designed according to these principles is unstable, one or more other parameters or guidelines described herein may be considered to produce a more stable insert.

In some examples, an active or targeting sequence may extend into the apical loop or across the apical loop from one side of the insert to the other. However, this type of structure may not be possible for all targeting sequences, and might not be beneficial (despite the potentially increased length of targeting sequence) with all targeting sequences, i.e. siRNA targeting sequences.

The limit on the APE of a base-paired region helps to avoid clustering of high positional entropy bases, e.g. bases with positional entropy of more than 1.0. Although the presence of individual high positonal entropy bases may not result in a high APE, inserts are less stable when the high positional entropy bases are numerous or clustered near each other. Other factors that may be derived, for example from an analysis of wild type hairpin-like structures and/or examples of stable exogenous hairpin-like structures, to indicate clustering include, for example, the percentage of nucleotides with a high positional entropy (e.g. a positional entropy greater than 1.0); the standard deviation of positional entropies, and the number of bases with very high positional entropy.

In some embodiments, the heterologous segment is inserted in the location of, and as a replacement for, a hairpin or hairpin-like structure that the heterologous element mimics in the wild type virus. In some embodiments, the heterologous segent is inserted in the location of, and as a replacement for, a hairpin or hairpin-like structure that the heterologous element does not mimic in the wild type virus. In some embodiments, the heterologous segment is inserted at a location that previously did not have any hairpin or hairpin-like structure in the wild type virus.

The present disclosure is also related to a method of making a viral vector, and to a plant or animal infected with a non-naturally occurring population of a virus, to a method of introducing a therapeutic agent into a plant or animal, to the use of a vector to treat a plant or animal, and to a method of treating a disease of a plant or animal.

In some embodiments, the heterologous segment includes an RNA bioactive molecule. The RNA bioactive molecules may be separated from progeny of the vector in the plant or remain part of progeny of the vector. In some embodiments, the vector may be used as a virus induced gene silencing (VIGS) vector or in a manner analogous to a VIGS vector, for example wherein small RNA segments such as siRNA generated from the heterologous segment of the vector interact with the host plant or a pathogen. In some embodiments, the small RNA segment, or a derivative thereof produced in the plant, interacts with a pathogenic virus to prevent replication of the virus rather than binding to plant cellular mRNA to silence expression of a host gene, or there is cross-kingdom transfer into a pathogen to target expression of a pathogen gene. The vector optionally replicates in the host plant. In some embodiments, progeny of the vector made in host plant cells enter the phloem where they may be transported long distances to other phloem-associated cells. In some embodiments, the progeny may be degraded by the host plant's DICER mechanism into small RNA segments including some or all of the heterologous RNA segment inserted into the vector. These heterologous small RNA segments may include an RNA bioactive molecule, for example siRNA which interfere with expression of a gene of the host plant or interfere with expression of a gene of a plant pathogen.

siRNAs can be used to target bacteria in plants, for example the Candidus Liberibacter asiaticus (CLas) bacteria. Plant pathogenic bacteria can be targeted using siRNAs that are produced in plants, taken up by the bacteria, and directly reprogram gene expression in the bacteria as described for example by Singla-Rastogi et al. (2019) Plant small RNA species direct gene silencing in pathogenic bacteria as well as disease protection, bioRxiv preprint post, Dec. 3, 2019, doi: https://www.biorxiv. org/content/10.1101/863902v1. In some implementations, CY1 or another ULV based vector is provided that contains siRNA hairpins that target a bacteria such as Candidus liberibacter asiaticus and render the bacteria non-pathogenic. For example, an siRNA hairpin provided to a plant by a ULV based vector may be taken up the CLas or another bacteria in the plant and control gene expression in the bacteria, thereby killing the bacteria and/or inhibiting an increase of the bacterial population.

It is commonly believed that bacteria do not take up siRNA. However, as mentioned above, Singla-Rastogi et al. (2019) describes examples in which small interfering RNA targeted against some specific genes were taken up by Pseudomonas syringae and cause a 50% reduction in the population of Pseudomonas syringae. Small RNA are also taken up by other bacteria. In particular, bacteria are taken up by Xylella, Erwinia amylovora and Liberibacter crescens. These three bacteria are all gram negative bacteria that infect plants. However, since these three bacteria are otherwise unrelated to each other, they indicate that small RNA can be taken up by bacteria that infect plants generally, or at least by gram negative bacteria that infect plants. P. syringae is a plant pathogen that causes, for example, bacterial canker in almond trees. Erwinia amylovora is a plant pathogen that causes, for example, fire blight in apple trees, pear trees and some other trees in the Rosaceae family. Liberibacter crescens is a relative of Liberibacter asiaticus and, based on their experiments with Liberibacter crescens, the inventors believe that Liberibacter asiaticus will also take up small RNA.

An siRNA is typically designed to be a complement to a part of RNA or DNA associated with the target organism intended to be treated or controlled by the siRNA (“specific siRNA”). For example, Xylella, Pseudomonas syringae, Erwinia amylovora and Liberibacter crescens can all be controlled by small RNA that are complements of genes (including complements of messenger RNA) of the bacteria. In particular, these bacteria were controlled, for example by 1000 fold reductions in their population in infected plants, by specific siRNA that complement the adenylate kinase (ADK) or gyrase subunit A (GyrA) genes of the bacteria.

The present disclosure is also related to a vector comprising a hairpin-like heterologous segment targeted against a plant susceptibility gene (S-gene). The vector may be derived from a ULV, CTV or or TRV. The S-gene may be, for example, MLO6 or other S-genes, such as DMR6, DND1 or ML07.

Optionally, the heterologous segment of an RNA vector may contain a non-coding RNA insert that is therapeutic either as part of the vector, when separated from the vector, or in progeny of the vector, but does not produce siRNA.

Optionally, the heterologous segment of an RNA vector may contain a coding insert, for example an insert that encodes a peptide such as an antimicrobial peptide.

In some embodiments, the action of the vector is primarily related to replication of the vector in the host plant cell rather than, for example, the stable transfection of the host plant genome. The vector is not a stable transfection vector although infection by the vector may be initiated by a transient transfection. The vector does not produce a recombinant host. The host does not express a heterologous insert of the vector by way of RNA or peptide expression of a modified host genome.

The heterologous segment of the vector can include an RNA bioactive molecule that is separated in the host plant, for example by way of a DICER mechanism, from the vector before having a therapeutic effect. While the viral vector may be limited to the phloem, a separated RNA bioactive molecule may move into and out of the phloem. Alternatively, the RNA bioactive molecule may have a therapeutic effect while part of progeny of the vector.

The heterologous inserts are designed to have an effect related to the health of the host plant or animal. For example, a heterologous insert may be targeted against the activity of a pathogen or the activity of a gene of a host plant involved in responding to a pathogen. The word heterologous does not necessarily require a sequence derived from another organism. However, in various examples a heterologous (or exogenous) RNA may include an active portion similar to (or similar to a complement of) an RNA in, or produced by, another organism. For example, the heterologous insert may target RNA produced by another organism. Similarly, a heterologous (or exogenous) segment may encode a peptide that is similar to a peptide, or a portion of a peptide, normally produced by another organism. In this context, the word “similar” may mean having a sequence identity of at least 70%, at least 80% or at least 90% with the reference sequence, being able to base-pair with the reference sequence, and/or having substantially the same activity as the reference sequence.

Also provided herein is the heterologous segment, such as the hairpin-like segment, disclosed herein. In some embodiments, the hairpin-like segment comprises, a first side, a second side, and an apical loop between the first side and the second side, wherein the first side has an a active portion and a sequence of the second side being less than 90% or less than 85% but more than 65% or more than 75% complementary with a sequence of the first side.

In some embodiments, the hairpin-like segment has one or more of:
a) 19 or less base pairs or 17 or less base pairs or 13 or less base pairs or 10 or less base pairs in base-paired regions of the hairpin-like segment,
b) no more than 4, or no more than 3, consecutive G:C base pairs in the hairpin-like segment,
c) no more than 4 consecutive A:U base pairs in the hairpin-like segment, d) no more than 21, or nor more than 20, bases cumulatively on both sides of non-base paired regions of the hairpin-like segment, and
e) between 40 and 300, 40 and 200, 60 and 300 or 60 and 200 bases.

In some embodiments, the hairpin-like segment disclosed herein is inserted into an RNA segment derived from a virus described herein, for example a segment from a ULV, CTV or TRV.

Further provided herein is a ribonucleic acid (RNA) molecule derived from an RNA virus whose nucleotide sequence has been modified to comprise a heterologous RNA segment, wherein said heterologous RNA segment: (A) comprises a length of between about 40-300 nucleotides; and, (B) is capable of forming a hairpin-like structure, wherein:
(1) the nucleotides of said hairpin-like structure exhibit an Average Positional Entropy (APE) similar to the APE of one or more naturally occurring hairpin-like structure of said virus or another RNA virus; and/or
(2) said hairpin-like structure exhibits a minimum free energy (AG) relative to length that is within 10 kcal/mol, or within a range between-5 and +15 kcal/mol, or within 20 kcal/mol for an insert of 150 nucleotrides or more, of the minimum free energy relative to length exhibited by said one or more naturally occurring hairpin-like structures of said virus.

In some embodiments, the heterologous segment has a minimum free energy within 10 kcal/mol, or within a range between-5 and +15 kcal/mol, or within 20 kcal/mol for an inseret of 150 nucleotides or more, of a naturally occurring hairpin-like structure of said virus that is within 10% of the size of the heterologous segment.

In some embodiments, the heterologous segment has a minimum free energy within 10 kcal/mol, or within a range between-5 and +15 kcal/mol, or within 20 kcal/mol for an insert of 150 nucleotides or more, of the minimum free energy predicted by the length of the heterologous segment and a line of best fit though a plot of minimum free energy vs. length for a plurality of naturally occurring hairpin-like structures of said virus.

The vector may be derived from a virus. In some embodiments, the virus is a plus-sense RNA plant virus. In some embodiments, the virus is a ULV, CTV or TRV. In other embodiments, the virus is an RNA animal virus, such as poliovirus.

Additional characteristics and features of the present disclosure will be further understood through reference to the following additional examples and discussion, which are provided by way of further illustration and are not intended to be limiting of the present disclosure.

### EXAMPLES

To generate the first CY1 vector, selective 2′-hydroxyl acylation analyzed by primer extension (SHAPE) RNA structure probing and phylogenetic structure comparisons were used to solve the secondary structure of full-length CY1 (2.7 kb). Once the structure was known, the 3′UTR was deletion mapped to find sites that did not disrupt important functions. Using a model plant that is a host for most viruses (Nicotiana benthamiana) and infecting plants via Agrobacteria tumefaciens-mediated vacuum infiltration, the stability of CY1 with hairpin inserts in various compatible locations was determined using RT-PCR and direct sequencing. Stability may be measured by the period of time post infection until the majority of CY1 accumulating in the plant are missing all or part of the insert. The period of stability required may vary with the application.

In order to determine where inserts are tolerated downstream of the sgRNA promoter in CY1, an evaluation of where critical elements exist in the 3′ UTR of CY1 was conducted, so that such elements are avoided when inserting heterologous sequences. The 3′ CITE for CY1 was identified, as well as several additional 3′ proximal hairpins that are highly conserved in umbraviruses and known to be critical for replication and translation. Using deletions/point mutations, the sequence from the start of an sgRNA promoter-like region (positions 2068-2092 of SEQ ID NO: 1) and upstream of the 3′ CITE (positions 2468-2551 of SEQ ID NO: 1) was investigated for regions that do not materially impact either accumulation in protoplasts or systemic movement in N. benthamiana. A similar strategy was previously utilized by the present inventors to identify regions in the 3′ UTR of TCV that can accommodate hairpins targeted by RNase III-type enzymes (Aguado, L. C. et al. (2017). Rnase III nucleases from diverse kingdoms serve as antiviral effectors. Nature 547:114-117).

Inserts in CY1 may be located at one or more of positions 2083 (optionally preceded by a STOP codon) 2250, 2301, 2304, 2317, 2319, 2330, 2331, 2336, 2372, 2375 and 2426. All of these locations are in the 3′ UTR from the start of an sgRNA promoter (or promoter-like) region, typically resembling the carmovirus consensus sequence (CCS: GGGUAAAAUA) but optionally with one or two base substitutions, to the start of a 3′ Cap Independent Translation Enhancer (3′ CITE). These position numbers are in relation to SEQ ID NO: 1. Deletions or substitutions may also be tolerated in this region. The deletions or substitutions may include replacement of entire hairpin-like in or overlapping with this region with hairpin like regions designed according to “mimic” principles as described elsewhere in this application or in International Publication Number WO 2023/215782, RNA Vectors with Hairpin-like Inserts, published on Nov. 9, 2023. In CY1, for example, the hairpin-like regions at 1962-2134 or 2220-2280 may be replaced with heterologous segments.

Inserts, deletions or substitutions are expected to be stable in analogous or homologous positions in other ULVs with reference to analogous positions in CY1, for example by comparing the secondary structure of the ULV with the secondary structure of CY1. Since most ULV have expanded 3′ UTR relative to CY1, additional insert, deletion or substitution locations may be present in other ULVs. Techniques similar to those described herein may be used to locate insert, deletion or substitution positions in other ULV.

The identification of the region described above is not intended to preclude the possibility of a vector having inserts, deletions or substitutions in other parts of the ULV backbone.

### Introduction to Development of Hairpin-like Inserts

Before factors involved in insert instability could be understood, more information was needed on the basic biology of CY1 and other ULVs. Like many viruses, ULVs use-1 ribosomal frameshifting for synthesis of their RdRp. In vitro translation using wheat germ extracts revealed an astonishing 30% frameshifting rate for CY1, compared with 2 to 5% for other members of the Tombusviridae. It was hypothesized that the high frameshifting rate for CY1 is required to synthesize a large number of progeny given the absence of a protecting RNA silencing suppressor. Moreover, all CY1 containing unstable inserts had reduced frameshifting levels, even though the frameshift site is located 1.7 kb upstream of the nearest insertion site. In addition, a correlation was found between mostly stable inserts and more modest reductions in frameshifting rates. When mapping sequences important for the high rate of frameshifting, critical sequences were found that spanned over 80% (2.2 kb) of the CY1 genome, with four long-distance tertiary interactions and two proximal tertiary interactions connecting local and distal sequences with the recoding site. These interactions contributed to multiple, highly conserved alternative RNA conformations in the recoding region. Initial efforts to increase stability of constructs with hairpin inserts centered on enhancing the stability of the active conformation of the recoding site, and while stability of inserts was significantly enhanced, low levels of deleted variants were still detectable by RT-PCR. To understand why inserts were affecting frameshifting, high-throughput SHAPE RNA structure probing of full-length WT CY1 and CY1 with inserts of varying stabilities were compared. Even small, fully base-paired hairpins caused substantial flexibility changes in residues throughout the entire genome, with unstable hairpins causing significantly more flexibility changes than ones that were mostly stable. Many flexibility changes were located in unpaired internal and terminal loops and in key RNA structures, including those at the recoding site, the cap-independent translation enhancer located in the 3′UTR and a highly conserved Y-shaped structure upstream of the recoding site that participates in one of the tertiary interactions. These results led to one of two paradigm-shifting hypotheses to explain why foreign hairpins in RNA virus vectors are not entirely stable: The conformation of the entire CY1 genome has evolved to be metastable and highly interactive through canonical and non-canonical tertiary interactions, and hairpin-like structures throughout the dynamic genome structure are perfectly tuned thermodynamically to maintain the genome-wide conformation required for critical virus functions. In other words, natural hairpin-like structures in the genome “backbone” are evolved to maintain a genome-wide metastable conformation. Insertion of a foreign (and thus non-optimized) hairpin stresses this integrative structure, leading to cascading destabilization that impacts critical tertiary interactions necessary to perform required functions such as frameshifting. The natural ability of error prone, non-processive RdRp to delete all or part of the interfering structure re-establishes viral genome homeostasis and thus enhances fitness.

To begin testing this hypothesis, exact duplicates of four natural CY1 hairpin-like structures from coding and non-coding regions were inserted into 3′UTR locations that had “kicked out” some foreign hairpins. These hairpin-like structures ranged in size from 33 to 198 nt (labeled 1 to 4 in FIG. 39). Stability of the vectors after 5 to 8 weeks in plants was measured by: (1) RT-PCR using primers outside of the insert; (2) batch sequencing the vector in newly emerged systemic leaves; and (3) direct sequencing of up to 10 cloned CYVaV1 vectors (FIG. 11). In every case, vectors containing any of these four duplicated hairpin-like structures were completely stable and no insert loss was detected. Next hairpin-like structures were designed that would “mimic” structures 1 and 4 for insertion in the vector. This involved generating hairpin-like structures with different sequences while maintaining elements of the secondary structure or conformation (i.e., the positions of paired bases and loops, apical loop size) and approximate minimum free energy (delta G or ΔG) (as measured by RNA folding programs) of the hairpin being mimicked. Designed hairpin-like structures typically had an experimental targeting sequence (the one desired in the RISC complex) on the 5′ side of the hairpin-like structure, and 3′ side nucleotides that would fulfil the desired shape and ΔG parameters. In one exception a targeting sequence extended from a region of the 5′ side, through the apical loop, to a region of the 3′ side. Of 52 vectors containing different mimic hairpin-like structures that were tested for stability in N. benthamiana, 50 were completely stable at the assayed time point (5 to 8 weeks after infiltration). Indeed, one such insert was found to still be stable after over a year in citrus (FIG. 11).

It was next endeavored to define properties associated with insert stability. The four natural inserts that were stable (which we used as models for mimicked inserts); four non-mimic inserts that were unstable; one non-mimic sequence of 198 nt that was unstable; 50 mimic hairpin-like structures that were stable; and two mimic structures that was unstable were examined. All stable hairpin-like structures were composed of nucleotides that together gave a low, but non-zero, average positional entropy (APE) value for the hairpin (FIG. 12). The formula for calculating the positional entropy of an individual nucleotide (i) is S(i)=−Σpᵢⱼ log (pᵢⱼ). The positional entropy of each nucleotide in a naturally occurring or exogenous sequence can be determined, for example, using the perl script “replot.pl” from the RNAfold program in the Vienna RNA package (https://www.tbi.univie.ca.at/RNA/). The APE is calculated by adding the positional entropy of the individual nucleotides (e.g. as extracted from the EPS file produced by the RNAfold program) and dividing by the number of nucleotides. For naturally occuring hairpin-like structures in CY1, individual nucleotide entropy values vary from 0 to 2.6, where nucleotides that are always predicted to be either unpaired or paired with the same partner have a positional entropy value of 0 and nucleotides with more than one possible pairing partner, or that may be paired or un-paired, have higher entropy values. Positional entropy values were extracted for all nucleotides present in each CY1 segment of interest using the RNAFold web server. Individual nucleotides were color coded from white (entropy value=0) to darkest gray (entropy value=2.6) (FIG. 12, Panel A). Positional information was then sorted and the average positional entropy (APE) for each hairpin-like structure was determined (FIG. 12, Panel B) with the following results.

The four natural model hairpin-like structures that were all stable had APE values of: 0.19 (Hairpin-like structure 1); 0.10 (Hairpin-like structure 2); 0.12 (Hairpin-like structure 3); and 0.15 (Hairpin-like structure 4). All 50 stable mimic hairpin-like structures had APE values ranging from 0.07 to 0.32, which was substantially lower than nearly all of the unstable inserts. Most of the stable hairpin-like exogenous segments also had minimum free energy (DG) similar to (i.e within 10 of) a naturally occurring hairpin-like structure in CY1 of similar (e.g. within 10%) length.

Four unstable hairpin-like inserts had APE values of 1.3, 1.16, 0.83, and 0.39. The unstable insert with APE=0.39 (referred to as M2250gfp30ext herein) was noted to have a 9 high positional entropy (PE greater than 1.0) and 4 very high positional entropy (PE greater than 1.5) nucleotides, with 8 (out of 66 nucleotides total) clustered near the apical loop, which may have contributed to the lack of stability. A deleted (stable) variant found accumulating in plants had a deletion of the apical loop, allowing the high positional entropy residues to form a new apical loop. Another unstable insert (198 nt) was not designed to assume any particular structure and had an APE value of 0.90. An unstable insert with low APE value (0.07; two asterisks in FIG. 12, Panel B) had three consecutive G:C pairs at the stem base and five consecutive G:C pairs in the apical region. Deleting 2 nt from the 3′ side of this hairpin (i.e., deletions did not affect the target sequence on the 5′ side) converted the hairpin from unstable to stable (APE increased slightly to 0.15).

These results strongly suggest that insert APE, optionally together with insert AG relative to the insert size, represent important parameters for designing stable inserts. Furthermore, results may be optionally improved by considering one or more additional stability parameters, for example the number of consecutive G:C pairs and number or clustering of high entropy nucleotides. Without intending to be limited by theory, these findings led to the second hypothesis to explain insert instability of VIGS vectors: High hairpin APE values, which imply that the hairpin is metastable and likely assuming multiple conformations with similar high ΔG values negatively impacts the processivity of the RdRp leading to polymerase pausing, which enhances the probability of a recombination event that excises some or all of the insert, thereby leading to unstable inserts. Without intending to be limited by theory, the overarching hypothesis then can be summarized as follows: Hairpin-like structures in viral genomes have evolved to have low APE values, possibly along with specific ΔG relative to the size of the hairpin-like structure, to maintain the tertiary structure of the metastable full-length genome and processivity of the replicating polymerase. Heterologous segments inserted into a viral vector must conform to these properties, which may or may not be virus-specific, to maintain viral vector fitness.

While vector stability for replication is also important in many applications, the ability to design stable inserts for virus vectors is important for allowing VIGS to be used in long-lived plants (e.g. trees and vines) for extended times against pathogens.

It is expected that the parameters for stable inserts described herein extend to inserts used in other RNA vectors derived from other viruses. Very little is known about the secondary structure of most viruses. For all but three viruses, the secondary structure is known for only portions of the virus. However, to the extent that secondary structures are known, naturally occurring viruses generally do not contain long fully based-paired regions. Considering the experiments and observations described herein, it seems likely that stable inserts for other virus-derived vectors will follow the principles described herein. In the event that one or more numerical ranges described herein do not apply to vectors derived from another virus, it is expected that the general technique of mimicking a natural-occurring hairpin-like structure will be useful. For example, the ΔG relative to length and/or APE and/or secondary structure of a wild-type hairpin-like structure may be used as a guide to designing a stable insert for a vector derived from the same wild-type virus. Given the similarities that have been observed between different Class 2 ULV, and the stability of a hairpin-like structure from one Class 2 ULV (OULV) when inserted into another Class 2 ULV (CY1), it is expected both the general technique of mimicking and the numerical parameters described herein to apply throughout Class 2 ULV. For example, it is expected that an insert described herein that is stable in CY1 will also be stable when inserted at least into any other ULV or any other Class 2 ULV. Further, given the observations and examples described herein regarding TRV and CTV, it is expected that an insert described herein that is stable in CY1 will also be stable when inserted at least ino CTV and TRV.

The secondary structure of RNA hairpin-like structures includes one or more of base-paired stacks, internal loops (symmetric or asymmetric), apical loops, and junctions (FIG. 13). It is hypothesized that thermodynamic features of hairpin-like and other structures contribute to the stability of the entire CY1 genome, and structures of natural hairpin-like and other regions reflect the end point of long-term thermodynamic evolution. By understanding parameters that have influenced virus secondary structure evolution, foreign hairpin-like structures can be designed with similar features for insertion into virus vectors without loss of fitness or RdRp processivity that otherwise would lead to insert loss.

Solving the secondary structure of CY1 revealed: (1) the longest fully base-paired region was found in Structure 2 and has 13 continuous base pairs. Structure 2 also has the largest (7 base) apical loop and smallest DG (−13.1 kcal/mol) (FIG. 12), (2) the length of the longest hairpin-like structure is 198 nt (Structure 1); (3) the length of the longest sequence without local Watson-Crick (canonical) or Watson-Crick (wobble) base-pairing is 21 nt (this sequence is not part of a hairpin-like structure); and (4) the highest number of consecutive G:C pairs in a stack in the CYVaV1 genome is three. Moreover, as described above, the APE of four natural hairpin-like sequences that were studied ranged from 0.10 to 0.19, and the APE of stable hairpin-like mimics ranged from 0.07 to 0.32, values that were lower than those of nearly all unstable inserts. As used herein, the term “base-paired” includes Watson-Crick (canonical) base pairs (i.e. C: G and A:U pairs) and Watson-Crick (wobble) base pairs (i.e. G:U base pairs) when G:U pairing is predicted by the minimum free energy structure.

One hairpin-like insert with a comparatively low APE (GFPmmck63, APE of 0.25) was unstable. However, this insert had five continuous G-C pairs. Another hairpin-like structure with a comparatively low APE (M2250gfp30ext, APE of 0.39) was also unstable. However, this insert had 9 bases (13.8%) with high entropy (PE greater than 1.0), 4 bases with PE over 1.5, and a standard deviation of entropy of 0.52. The instability of these examples may not indicate the maximum APE that may results in a stable hairpin-like insert. A hairpin-like insert with a similar or higher APE, but without the continuous G-C pairs, large percentage of high entropy bases and/or high standard deviation of PE, might be stable. Stable hairpin-like inserts may have a maximum APE of 0.75, 0.65. 0.5, 0.4, 0.39, 0.36 or 0.32. Mimimum APE of a stable hairpin-like structure may be 0.01, 0.02, 0.03, 0.04, 0.05, 0.06 or 0.07. A range of APE may optionally be provided by any combination of these minimum and maximum APE values, or any APE values described in the examples.

An insert may exceed one or more of the parameters described above or derived from a wild type virus. For example, one hairpin-like mimic was stacked onto the majority of Structure 3 (in its natural location), resulting in a stable hairpin-like structure of 247 nt. The entirety of this hairpin-like structure was not heterologous, however it suggests that the maximum length of a natural hairpin-like structure might be exceeded. A stable insert with 4 consecutive G:C pairs was also introduced into CY1, whereas a maximum of 3 are found in natural CY1 hairpin-like structures. An insert with 5 consecutive G:C pairs was not stable. Further, heterologous inserts with APE greater than the APE of natural CYVaV hairpin-like structures were used. The examples above suggest that in some examples a natural parameter may be exceeded, for example by up to one half, or by up to one third.

U.S. Pat. No. 7,229,829, Tobacco rattle virus and related compositions and methids, which is incorporated herein by reference, describes RNA1 and RNA2 sequences of tobacco rattle virus (TRV). Other TRV vector sequences are also known. As is known in the art, TRV RNA1 and RNA2 sequences, or portions thereof, can be modified to include a heterologous RNA segment for delivery to a plant. to achieve, for example, host plant gene downregulation (i.e. as a VIGS vector). TRV vectors are used, for example, to test the effects of proposed genetic modifications of plants. However, in this context, the heterologous RNA segment is only required to remain in progeny of the vector for a short period of time. Folding the first 2400 nt of tobacco rattle virus (TRV) RNA2 revealed a low entropy hairpin-like region from positions 1727 to 1925. The APE value for this hairpin-like region is 0.32 and the ΔG is-61.5 kcal/mol. This can be compared to similarly sized Structure 1 of CY1 (APE=0.19; AG=−89.2 kcals/mol).

International Publication Number WO 2022/133186, Create Huanglongbing tolerance by silencing a citrus negative immune regulator, which is incorporated herein by reference, describes RNAi vectors (e.g. VIGS vectors) derived from citrus tristeza virus (CTV). A heterologous RNA segment is inserted in one of four regions of the CTV genome. In another example, a naturally occurring hairpin-like structure was located in CTV and inserted into CY1 and infected into a benthamina plant (see further below). The resulting vector was stable when assayed after three-weeks.

In combination with further examples related to poliovirus and coxsackievirus B3 (see further below), these observations suggest that inserts designed with reference to other wild type virus may be used in CY1 or other ULV, and that inserts designed with reference to CY1 or other ULV, or otherwise as described herein, may be useful in other viral molecules, for example other RNA molecules derived from plus-sense RNAviruses generally, other RNA plant virus generally, and other enteroviruses or other RNA animal viruses, as well as CTV, TRV, poliovirus and CXVB3 in particular. Even if the resulting vectors are not as stable as CY1-derived vectors, they may be more stable for replication than comparable vectors with conventional (i.e., fully base-paired) hairpins.

The inventors decided to test if inserting duplicates of natural hairpin-like structures into the insert sites would be stable. Despite the size of some of these structures, all four of the duplicate hairpin-like structures used were stable in the insert sites. Next, the inventors discovered that mimicking the secondary structures and approximate ΔG of the natural hairpin-like structures with heterologous sequences also resulted in vectors that did not lose the inserted sequences. The inventors then investigated the properties of the natural hairpin-like structures, finding that various parameters such as the positional entropy of individual nucleotides, average positional entropy of the overall hairpin, and AG were relevant to stability. Unstable inserts typically had either high positional entropy, higher AG, multiple consecutive G:C pairs, or high positional entropy nucleotides that were clustered together. Using these principles, exogenous inserts with targeting sequences were designed that could be inserted into vectors and that were stable (i.e. retained in progeny) even without any reverse fitness modifications described herein. However, one or more reverse fitness modifications may be optionally used in combination with otherwise stable inserts.

It is commonly believed that bacteria do not take up siRNA. However, Singla-Rastogi et al. (2019) describes examples in which small interfering RNA targeted against some specific genes were taken up by Pseudomonas syringae and cause a 50% reduction in the population of Pseudomonas syringae. The inventors have confirmed that the conventional belief is at least partially correct. For example, in experiments conducted by the inventors, E. coli did not take up siRNA. However, as described in the examples herein, small RNA are taken up by some bacteria. In particular, bacteria are taken up by Pseudomonas syringae, Erwinia amylovora and Liberibacter crescens. These three bacteria are all gram negative bacteria that infect plants. However, since these three bacteria are otherwise unrelated to each other, they indicate that small RNA can be taken up by bacteria that infect plants generally, or at least by gram negative bacteria that infect plants. P. syringae is a plant pathogen that causes, for example, bacterial canker in almond trees. Erwinia amylovora is a plant pathogen that causes, for example, fire blight in apple trees, pear trees and some other trees in the Rosaceae family. Liberibacter crescens is a relative of Liberibacter asiaticus and, based on their experiments with Liberibacter crescens, the inventors believe that Liberibacter asiaticus will also take up small RNA.

An siRNA is typically designed to be a complement to a part of RNA or DNA associated with the target organism intended to be treated or controlled by the siRNA (“specific siRNA”). For example, as shown in the examples herein, Pseudomonas syringae, Erwinia amylovora and Liberibacter crescens can all be controlled by small RNA that are complements of genes (including complements of messenger RNA) of the bacteria. In particular, these bacteria were controlled, for example by 1000 fold reductions in their population in infected plants, by specific siRNA that complement the adenylate kinase (ADK) or gyrase subunit A (GyrA) genes of the bacteria.

Sequences for growth inhibition of Erwinia amylovora in vitro and in vivo are presented below. Two Erwinia genes were targeted: MurA and GyrA.

Erwinia MurA (in vitro) (SEQ ID NO: 59):
gaactggtgaaaaccatgcgcgcctcgatttgggcattgggcccgctggtggcacgttttgg
ccaggggcaagtatcactgcccggtggttgcgctatcggcgcacggccggttgatcttcata
tcaccggccttgagcagctcggcgccgagatcaaactggaagaaggttacgttaaagcctct
gtcgcgggtcgcctgaaaggggcgcatatcgttatggataaggtcagcgtgggtgcaaccgt
cactatcatgagtgcggcgacgctggcaacgggcaccaccgttatcgagaatgctgcgcgtg
agccggaaattgtcgacactgccaacttcctcaacacgcttggggcgaaaatcaccggtgcc
ggcagcgatcgtatcaccatcgaaggtgttgatcgccttggtggcggtgtttatcgcgtact
tcctgaccgcatcgaaaccggtactttcctggtggcgggagcgatttccggcggtaaggtta
cctgccgtgcggcgcagcccgatacgctggatgctgtactggctaagctacgcgaagccggt
gcggacatcgagatgggagaagactggataagcctggacatgcacggtaagcggcctaaagc
ggtcaatttacgcacagcgccgcatcccggtttcccaaccgatatgcaggcgcagttcagtt
tgttgaacctggtggctgaaggcacgggggtgattactgaaaccatcttcgaaaaccgctt

Erwinia MurA (in vivo):

Fragment 1 (SEQ ID NO: 59, residues 1-402):
gaactggtgaaaaccatgcgcgcctcgatttgggcattgggcccgctggtggcacgttttgg
ccaggggcaagtatcactgcccggtggttgcgctatcggcgcacggccggttgatcttcata
tcaccggccttgagcagctcggcgccgagatcaaactggaagaaggttacgttaaagcctct
gtcgcgggtcgcctgaaaggggcgcatatcgttatggataaggtcagcgtgggtgcaaccgt
cactatcatgagtgcggcgacgctggcaacgggcaccaccgttatcgagaatgctgcgcgtg
agccggaaattgtcgacactgccaacttcctcaacacgcttggggcgaaaatcaccggtgcc
ggcagcgatcgtatcaccatcgaaggtgtt
Fragment 2 (SEQ ID NO: 59, residues 343-743):
aacacgcttggggcgaaaatcaccggtgccggcagcgatcgtatcaccatcgaaggtgttga
tcgccttggtggcggtgtttatcgcgtacttcctgaccgcatcgaaaccggtactttcctgg
tggcgggagcgatttccggcggtaaggttacctgccgtgcggcgcagcccgatacgctggat
gctgtactggctaagctacgcgaagccggtgcggacatcgagatgggagaagactggataag
cctggacatgcacggtaagcggcctaaagcggtcaatttacgcacagcgccgcatcccggtt
tcccaaccgatatgcaggcgcagttcagtttgttgaacctggtggctgaaggcacgggggtg
attactgaaaccatcttcgaaaaccgctt
Erwinia GyrA (in vitro) (SEQ ID NO: 60):
atcgtcaacctgctgccgctggaagccaacgagcgtattactgcaattctgccggtgcgtga
atatgccgaaggctggaatatctttatggctaccgccagcggaacggtgaagaaaaccgcgc
tgactgacttcagccgaccgcgcagcgccggtattattgccgtcaatctgcgtgatgacgat
gaactgatcggcgtgtcgctgacgaacggtagtgatgaagcgatgctgttttctgccgccgg
taaagtggtgcgattcgcggagagcgcggtgcgtacgatgggccgtaccgcctccggcgtac
gcggcatcaagctggctgaaggcgaccgcgtggtgtcgctgatcgtgccgcgtgatgatggc
gctatcatgaccgtgacgcaaaacggctacggtaaacgaacggctaacgtcgaatatcccac
caagtcgcgtgcgactcagggggttatctcgatcaaggtaaccgagcgtaacgggccggtta
tcggtgcggtgcaggtggtcgatggcgatcagatcatgatgatcaccgatgccggcacgctg
gtacgtacccgcgtgtccgaggtcagcgtggtagggcgtaacacccagg

Erwinia GyrA (in vivo):

Fragment 1 (SEQ ID NO: 60, residues 2-416):
tcgtcaacctgctgccgctggaagccaacgagcgtattactgcaattctgccggtgcgtgaa
tatgccgaaggctggaatatctttatggctaccgccagcggaacggtgaagaaaaccgcgct
gactgacttcagccgaccgcgcagcgccggtattattgccgtcaatctgcgtgatgacgatg
aactgatcggcgtgtcgctgacgaacggtagtgatgaagcgatgctgttttctgccgccggt
aaagtggtgcgattcgcggagagcgcggtgcgtacgatgggccgtaccgcctccggcgtacg
cggcatcaagctggctgaaggcgaccgcgtggtgtcgctgatcgtgccgcgtgatgatggcg
ctatcatgaccgtgacgcaaaacggctacggtaaacgaacggc
Fragment 2 (SEQ ID NO: 60, residues 207-607):
gacgaacggtagtgatgaagcgatgctgttttctgccgccggtaaagtggtgcgattcgcgg
agagcgcggtgcgtacgatgggccgtaccgcctccggcgtacgcggcatcaagctggctgaa
ggcgaccgcgtggtgtcgctgatcgtgccgcgtgatgatggcgctatcatgaccgtgacgca
aaacggctacggtaaacgaacggctaacgtcgaatatcccaccaagtcgcgtgcgactcagg
gggttatctcgatcaaggtaaccgagcgtaacgggccggttatcggtgcggtgcaggtggtc
gatggcgatcagatcatgatgatcaccgatgccggcacgctggtacgtacccgcgtgtccga
ggtcagcgtggtagggcgtaacacccagg

Gene sequences of P. syringae tabaci_Gyrase subunit A (Pst_GY), P. syringae tabaci_Adenylate Kinase (Pst-ADK), and GFPuv were utilized for specific siRNA targets are presented below (wherein sequences underlined in solid line are forward primers for dsRNA synthesis, and sequences underlined in dashed line are reverse primers for dsRNA synthesis):

P. syringae tabaci_Gyrase subunit A (Pst_GY) (SEQ ID NO: 61):
atgggcgaactggccaaagaaatcccccggtcaatatcgaagacgagctgaagcagtccta
cctcgactacgcgatgagcgtaatcgtcggtcgagcactgcccgatgcgcgcgacggcttga
agcccgtgcaccggcgcgtgttgttcgcaatgagcgagctgggtaacgactggaacaagccg
tacaagaaatccgcccgtgtggttggtgacqtgatcggtaagtatcacccgcacggcgatac
agccgtgtacgacaccatcgttcgtatggctcagccattctcgctacgctacctgctggtag
acggtcagggcaacttcggttcggtcgatggcgacaacgctgcggccatgcgatacaccgaa
gtgcgcatgaccaagctggcgcacgagctgctggccgacctgcacaaggaaaccgtggactg
ggtgccgaactacgacggcaccgaaatgatccccgcggtcatgccgacccgtattcccaacc
tgctggtcaacggttccagcggtatcgccgtgggcatggcgaccaacattccgccgcacaac
cttggtgaagtcatcgacggttgcctggcactgatcgacaaccccgagttgacgatcgatga
gctgatgcagtacatccccggcccggatttcccgacagcggcgatcatcaacggccgtgcgg

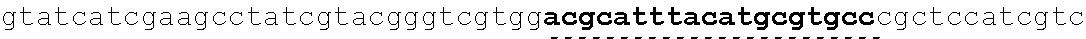

gaagacatcgacaaggtcggcggacgtcagcagatcgtcatcaccgaactgccgtaccagct
taacaaggcacgtctgatcgagaagatcgccgagctggtcaaggaaaagaaactcgaaggca
ttaccgagctgcgtgacgagtccgacaaggacggtatgcgcgtggtcatcgagctgcgtcgt
ggcgaagtgccagaggttgttctcaacaacctttatgcccagacccagctgcaaagcgtttt
cggtatcaacatcgttgccttgattgacggtcgtccacgcatcctgaacctcaaggacctgt
tggaagcctttgttcgtcaccgtcgcgaagtggttacccgccgtaccgtattcgagctgcgc
aaggcgcgtgagcgtggccatatccttgaaggtcaggctgttgcgttgtccaacatcgaccc
ggtcatcgccctcatcaaggcctctccgacacctgcagaagccaaggaggcgttgatcaaga
cgccttgggaatcaagcATGCGCGTGATTCTGCTAGGAgcagtggtcgaaatggtcgagcgt
gcaggtgccgattcgtgccgccctgagaacctggacccgcaatacggtctgcgtgaaggcaa
gtatttcctgtcaccggaacaggctcaggccattctggaactgcgtctgcatcgcctgaccg
gtctggaacacgagaagctgctgggcgagtaccaggaaatcctcaaccagatcggcgagctg
atccgcatcctcaacagcgcaacgcgcctgatggaagtgatccgcgaagagctggaagtgat
ccgctccgagtacggcgatgcccgtcgtaccgagattctcgatgcacgtctggacctgaccc
tgggtgacctgatcaccgaagaagagcgtgtggtcaccatctcccatggcggctatgccaag
acccagccattggcggtctatcaggctcagcgtcgtggggcaagggcaagtcggccaccgg
catcaaggatgaggattacattgctcacctgctggtcgccaacagccacacgacactgctga
tgttctccagcaagggcaaggtgtactggctcaagacctatgagatcccggaagcgtcccgc
gctgcccgtggtcgtccgttggtcaacctgttgccgctgagcgacggcgaatacatcaccac
catgctgccggttgacctcgaagccatgcgcaagcgtgccgacgaagaaggcgaagccctcg
aaggcgagctggacgacgcggaaaacagcagcgagaccgaagaagagcgcaaggcccgtatc
aaggccgccgacaagaagaaggctccgttcatcttcatgtccaccgccaacggtaccgtcaa
gaagaccccgctggttgcattcagccgtcaacgcagttcgggcctcattgcccttgagctgg
acgagggcgacatcctgatctccgctgccattaccgatggcgaacaggaaatcatgctgttc
tccgatggcggcaaagtgacccgcttcaaggaatccgatgtgcgcgccatggggcgtaccgc
tcgcggcgtgcgtggcatgcgtctgccagaagggcaaaagctcatttcgatgctgatcccgg
aagaaggcagccagatcctcaccgcttccgagcgcggttacggcaagcgtacggccatttcc
gagttccccgagtacaagcgcggcggtcagggtgtcatcgccatggtcagcaacgagcgtaa
cggccgtctggttggcgcagttcaggtgcttgatggcgaagaaatcatgctgatttccgatc
agggcacgctggtgcgtacccgggtgggcgaagtgtccagtctgggccgtaacactcagggt
gtgaccctgatcaagctggccagcgacgagaaactggtcggtctggagcgtgttcaggagcc
gtcggaagtcgaaggcgaagagcttgaaggcgaagaagttatcgacggcgtgattgtcgatg
ccgctgaagctgaagtgggcgacgccggtgaagacctgcaagcggacgctgcgccagacgaa
gacgaaccgcagaactga
P. syringae tabaci_Adenylate Kinase (Pst-ADK) (SEQ ID NO: 62):
atgcqcqtgattctgctaggagctcccggggccggtaaaggtactcaggcaaaattcatcac
tgaaaatttcggcatcccgcaggtttcgacaggcgacatgctgcgcgctgcagtcaaggctg
aaaccgagcttggcctgaaggccaagagcgtcatggactcgggtggtctggtttccgatgac
ctgatcattggtctgatcaaggatcgtctggcccagccggattgtgcgaacggcgttctgtt
cgacggcttcccgcgcaccattcctcaggccgaagccctgttgaaagcaggtctggaaatcg
accacgtgctggaaatcgccgttgatgacgaagaaatcgtcaagcgcatgtcgggccgccgg
gttcacgaaggctctggtcgcatctatcacaccattttcaacccgccgaaagtcgagggtat
cgatgatgtgactggtgaaccgctgttgcagcgcaaggacgacgtcgaagaaaccgtgcgtc
atcgcctgtcggtctaccatgcccagaccaagccgctggtcgagttctacagcaagctggaa
gcaaagaacggcaagcccaagtgcagccatattccaggtgttggctcggttgaagaaatcac

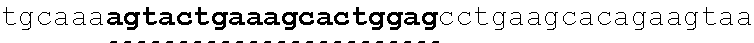

GFPuv (SEQ ID NO: 63):
atgagtaaaggagaagaacttttcactggagttgtcccaattcttgttgaattagatqgtga
tgttaatggqcacaaattttctgtcagtggagagggtgaaggtgatgcaacatacggaaaac
ttacccttaaatttatttgcactactggaaaactacctgttccatggccaacacttgtcact
actttctcttatggtgttcaatgcttttcccgttatccggatcatatgaaacggcatgactt
tttcaagagtgccatgcccgaaggttatgtacaggaacgcactatatctttcaaagatgacg
ggaactacaagacgcgtgctgaagtcaagtttgaaggtgatacccttgttaatcgtatcgag
ttaaaaggtattgattttaaagaagatggaaacattctcggacacaaactcgagtacaacta
taactcacacaatgtatacatcacggcagacaaacaaaagaatggaatcaaagctaacttca
aaattcgccacaacattgaagatggatcagttcaactagcagaccattatcaacaaaatact

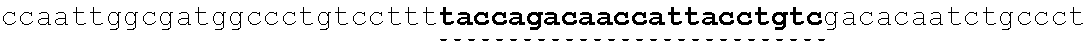

ttcgaaagatcccaacgaaaagcgtgaccacatggtccttcttgagtttgtaactgctgctg
ggattacacatggcatggatgagctctacaaataa

Enhanced Stabilization: The ability to achieve a sufficiently stable insert in a viral vector has eluded prior researchers due in part to a lack of expertise in RNA structure. Some researchers attempted to use an empirical trial-by-error approach to choose insert locations, but without a sufficient understanding of the effect of the insert on the stability of the vector. In accordance with the present disclosure, the core principles behind what makes inserts stable were investigated and utilized to optimize the process of insert location and structure. First, an excellent correlation was discovered between vector instability and numerous structural alterations throughout the viral genome as assayed by SHAPE structure probing, which results in poor in vitro translation of the polymerase that requires a −1 ribosomal frameshift. The following hypothesis was then tested: what if RNA viruses can discern “self” from “non-self”? In other words, what if RNA viruses have evolved their genomes such that every portion of the genome is maximally structured to support the most fit virus? This implies that every hairpin emanating from the genomic RNA nucleic acid “backbone” is maximally stable for its size and shape and properly configured for a successful virus.

It was determined that the insertion of haphazard hairpins whose design is based solely on siRNA generation, and which do not resemble in length, structure and/or stability endogenous, evolved hairpins, stresses and destabilizes the entire structure of a viral genome. Since RNA viral genomes are dynamic, with active and inactive structures precisely balanced to control key viral functions like frameshifting, destabilizing the genome can have adverse repercussions that are relieved if the inserts are deleted through recombination. If the genome contains foreign structures, the deletion or removal of inserts or portions thereof (e.g., replicase-mediated events) will eventually occur, thereby leading to the non-functionality of the commercial RNA vector product. Such deletion and/or removal of inserts or portions thereof increases the possibility of generating a more fit virus (though not necessarily functioning to target the desired pathogens/host gene expression), as the virus with a destabilizing insert is more poorly fit.

Following from the proposed hypothesis, designing inserts that “mimic” the structure (i.e., length and/or the size and location of looped or non-base-paired regions) and/or stability (i.e., the ΔG) of endogenous hairpins (which optionally include hairpin-like structures and structures with multiple hairpins or hairpin structures) should increase stability of the insert within the vector, particularly when inserted into suitable locations. Mimicked hairpins are designed by using the sequence of interest (i.e., an siRNA) as some or all of one side, for example the 5′ side, of the hairpin and an artificial sequence on the other side, for example the 3′ side, that provides the corresponding nucleotides for the base-pairs and loops, resulting in a structure that mimics an endogenous (natural) hairpin at some location on the wild-type vector or a related wild-type vector. A mimic insert may be inserted in the same location, i.e., as a replacement for the wild-type hairpin that was mimicked. Alternatively, a mimic insert may be inserted into a different location, with the wild-type hairpin either retained or deleted, if not required for viability of the vector.

In accordance with the present disclosure, hairpin inserts were engineered to have a structure that mimics the structure of a natural endogenous hairpin structure of the vector of interest or a relative thereof (e.g., a CY1 vector having an insert that mimics a hairpin structure of CY1 or another ULV, for example a Class 2 ULV such as OULV). As noted above, providing and maintaining stability of sequences inserted into the CY1 vector, which are subsequently converted into siRNAs when the RNA virus vector is targeted by the RNA silencing defense system in infected plants, raises various challenges. While some insertions are randomly more stable than others (i.e., the inserts remain in the vector for a longer time), little was previously understood about parameters required for stabilization. Solving the secondary structure of the CY1 vector revealed an extremely compact structure with few extensive single-stranded regions, which would generally be considered the most suitable locations for making insertions. Various locations (noted above) were identified for accepting particular hairpin inserts (e.g., that targeted nuclear-encoded GFP), although other hairpin structures had different stabilities in all of these particular locations. Thus, producing a vector that allows for little or no discernable insert loss (e.g., assayed first by PCR and then by sequencing the cloned population after four to six weeks) proved difficult. This indicated that these hairpins decreased the fitness of the vector and that deletion of almost all (but not precisely all as surrounding vector sequences could also be deleted) or part of the insert increased fitness.

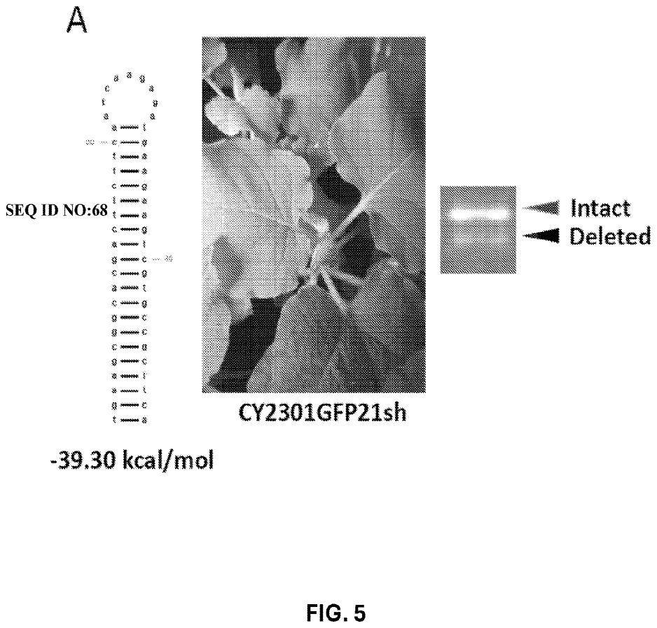

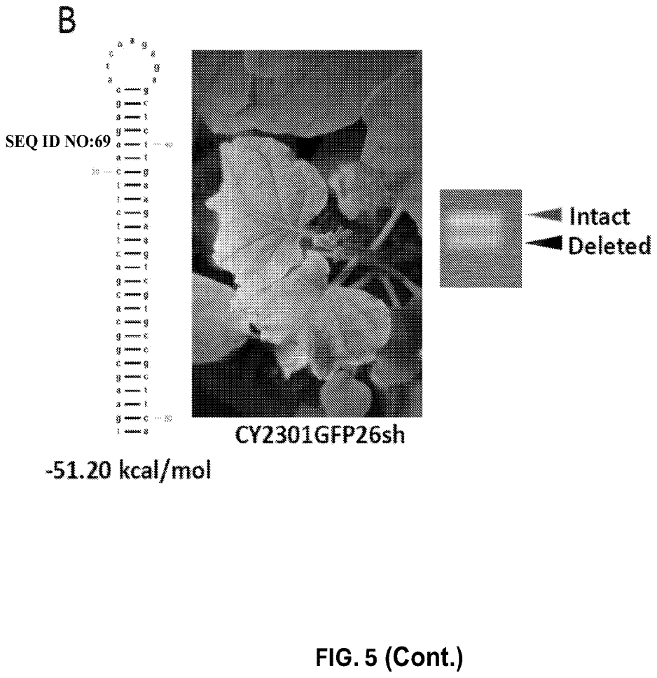

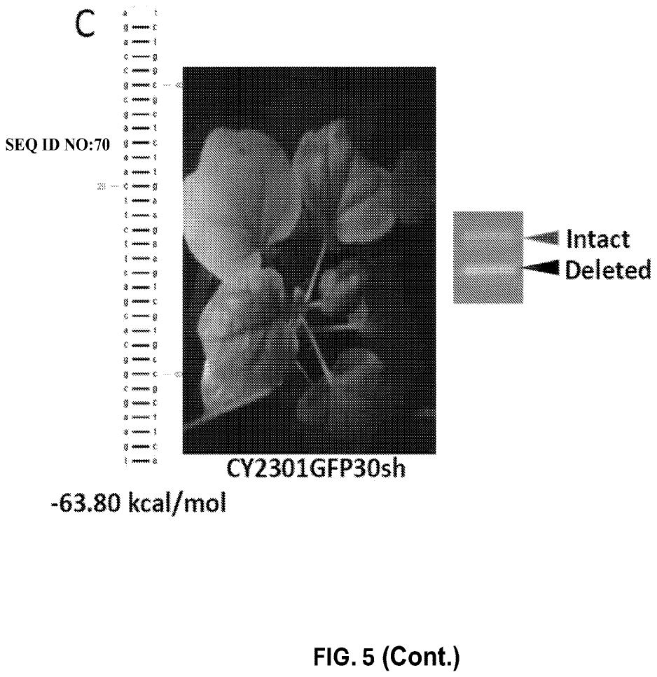

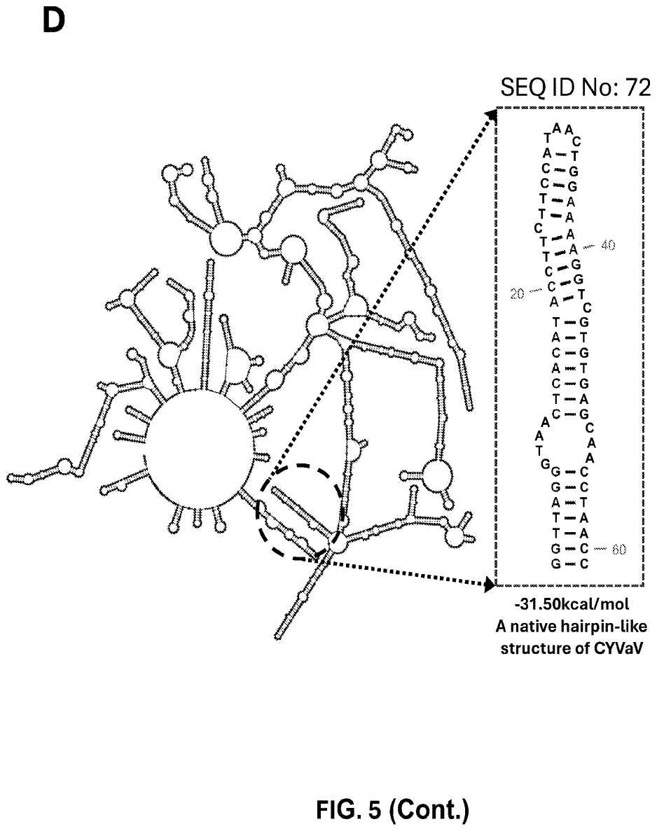

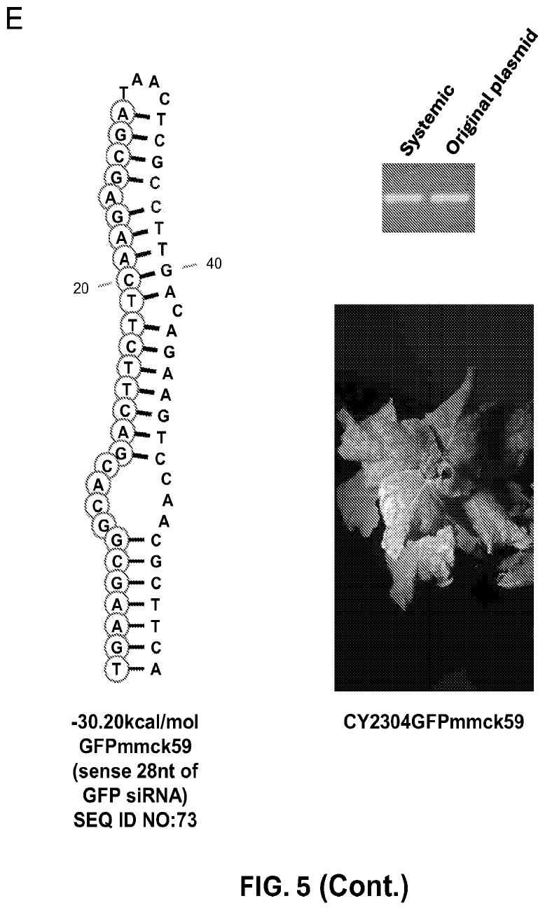

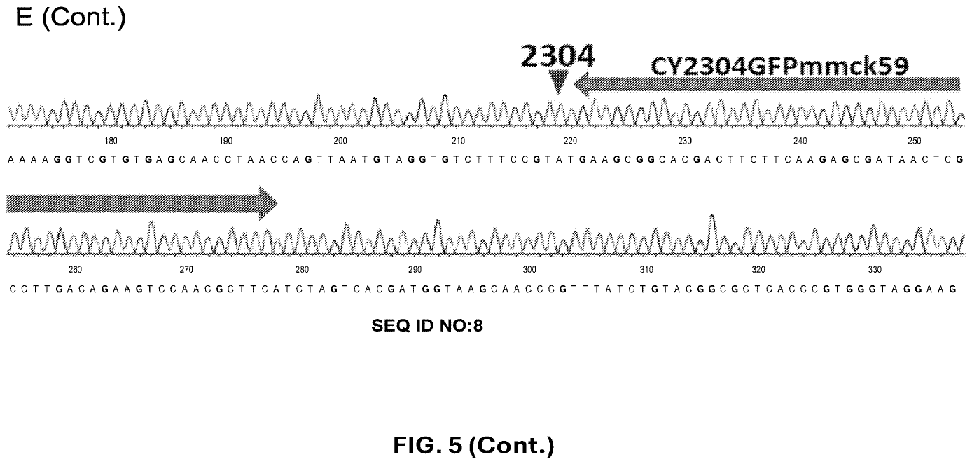

Intuitively, the more stable the structure of the hairpin insert (i.e., the higher or ‘more negative’ the AG), the less it should interfere with important viral structures (generally thought to be the reason for vector instability), providing that the polymerase is still able to melt the hairpin during replication. Surprisingly, it was discovered that the opposite is true (see FIG. 5, Panels A, B, and C). In particular, it was discovered that structurally stable hairpins were rapidly lost, as were inserted sequences with minimal structural stability. Also tested were non-structured inserts that were placed atop RNA scaffolds (e.g., lock and dock structures). While the lock and dock stabilized unstructured inserts and resulted in some very stable inserts, some inserts generated a small but discernable population of CYVaV that had lost various amounts of the insert and scaffold, particularly vectors with two such inserts.

An RNA vector (e.g., CY1 vector) including such mimic hairpins exhibited substantially increased stability as compared to a vector that included a hairpin structure that did not mimic the natural hairpin structure. The structure and sequence of a natural hairpin was determined; a mimic hairpin having a substantially similar structure and/or sequence and/or ΔG to the natural hairpin was then inserted into the vector molecule. Studies demonstrated that vectors possessing such mimic hairpin(s) inserted at the same location as the corresponding natural hairpin(s) (i.e., replacing the natural hairpin(s) at such location) were stable. Further studies demonstrated that vectors possessing such mimic hairpin(s) inserted at one or more different locations from the corresponding natural hairpin(s), but possessing substantially the same structure(s) and/or sequence(s) and/or ΔG as compared to the natural hairpin(s), were stable. It was found that hairpin structures having relatively conserved sequences across iRNAs (e.g., CY1 and relatives thereof, other RNA molecules having least 50% or at least 70% identity with CY1) were particularly suitable as mimic hairpin structures. However, it was also found that a wild-type hairpin structure in a CY1 relative, for example another Class 2 ULV such as OULV, could be used to design a stable CY1 insert even if the wild-type hairpin structure is not present in CY1.

Referring to FIG. 5, Panel E, a mimicked hairpin (wherein the natural yet unnecessary hairpin at position 2219 was replaced with a mimic hairpin that resembles the natural hairpin) produced an extremely stable virus vector with no discernable loss of insert for the life of the plant. For example, random events that may lead to insert loss may generate a less fit virus, with such virus(es) lost from the population. The process of replacing a natural hairpin with an engineered mimic hairpin was repeatedly tested, with several mimics for each of three different natural hairpins, including one that is fully base-paired, and the mimic hairpins inserted into positions 2219 and 2304. In every case, the mimicked hairpin replacement at positions 2219 and 2304 resulted in an extremely stable virus vector. It was determined that the closer the ΔG of the mimicked hairpin is to the endogenous hairpin being mimicked, the more stable the insert. RNA structure probing clearly showed far fewer structural disruptions throughout the vector genome from the presence of a mimic hairpin as compared to random hairpins inserted at the same locations. This further demonstrated the importance of mimicry to achieve a vector that maintains a structure close to (though generally not identical to) the wild-type RNA genome structure.

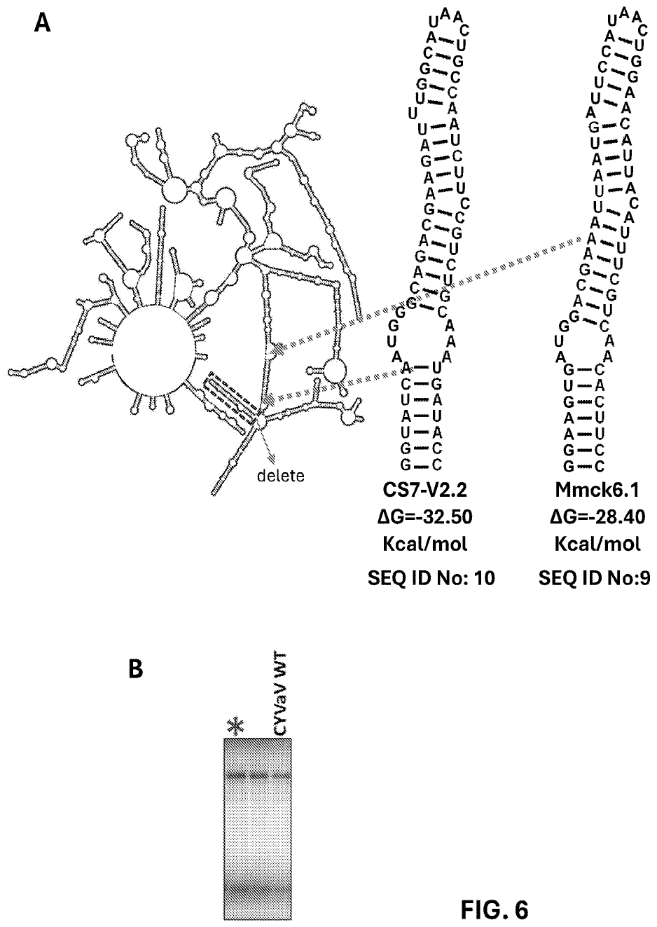

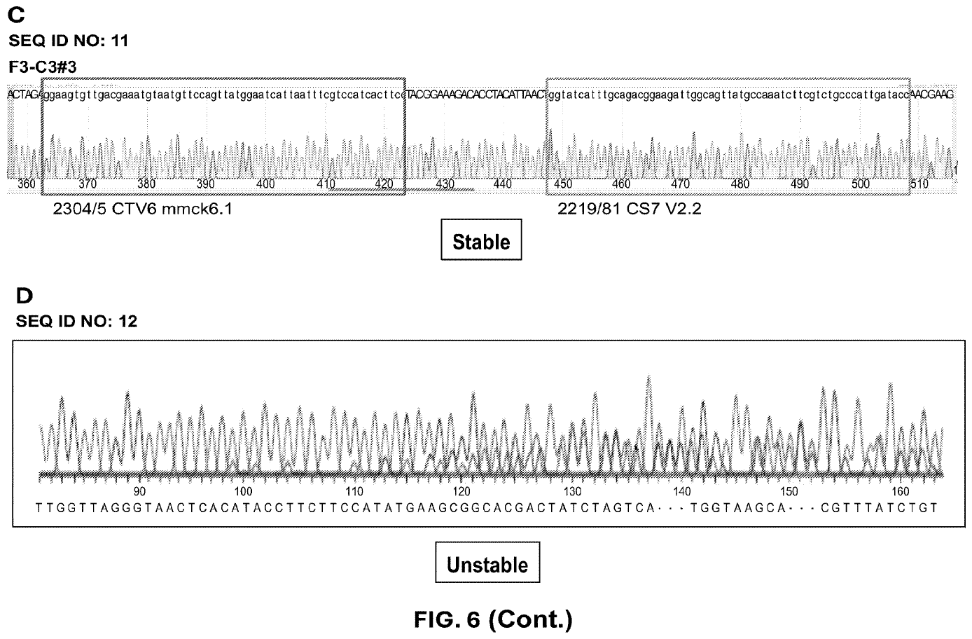

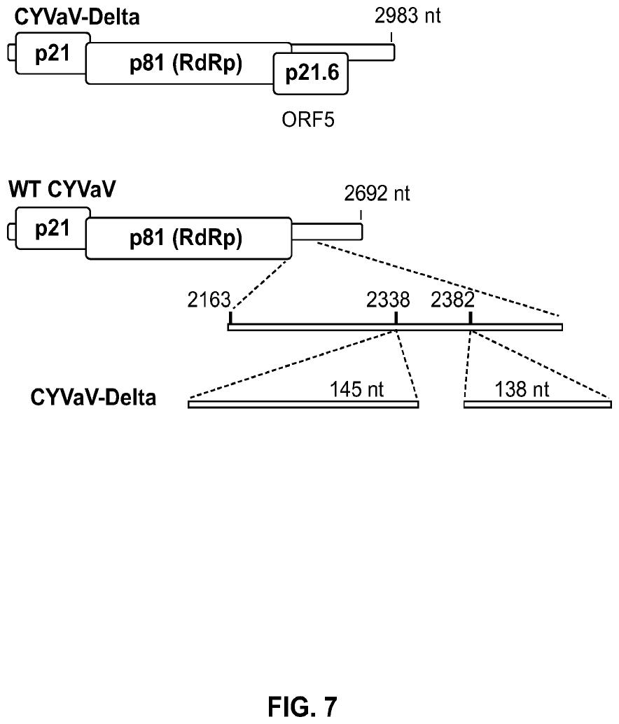

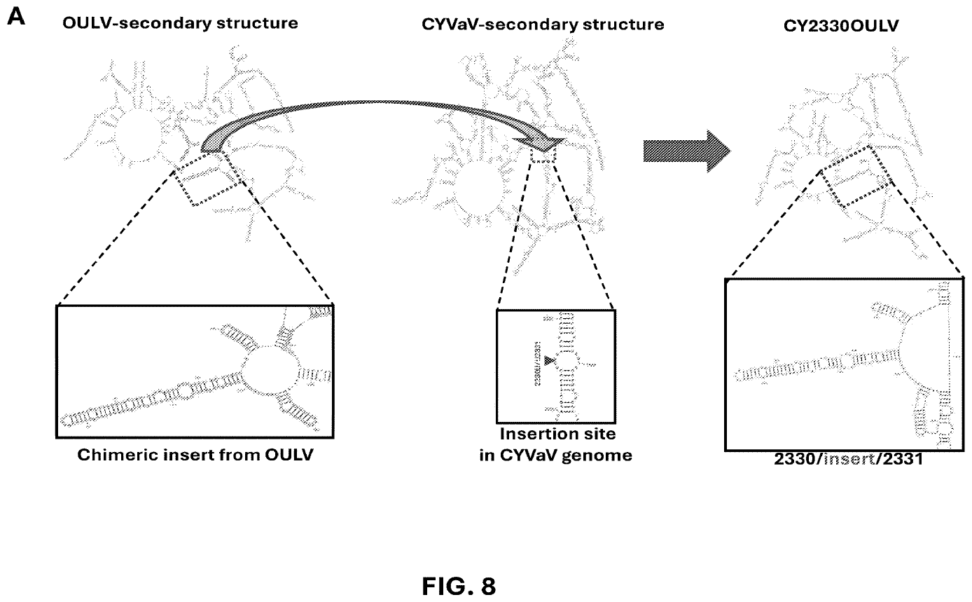

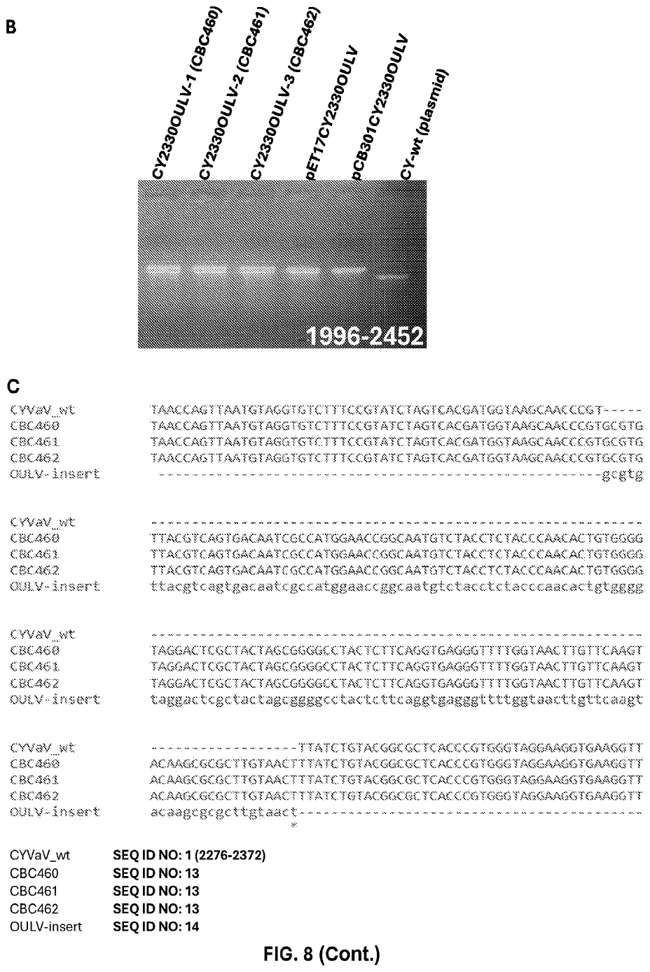

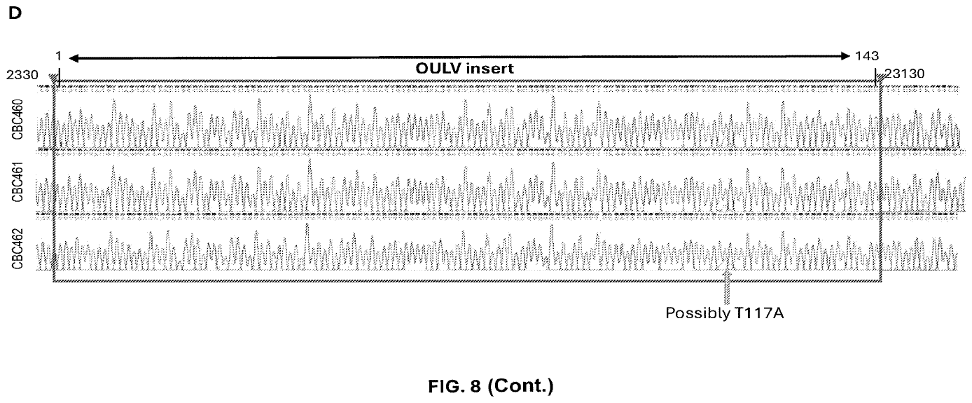

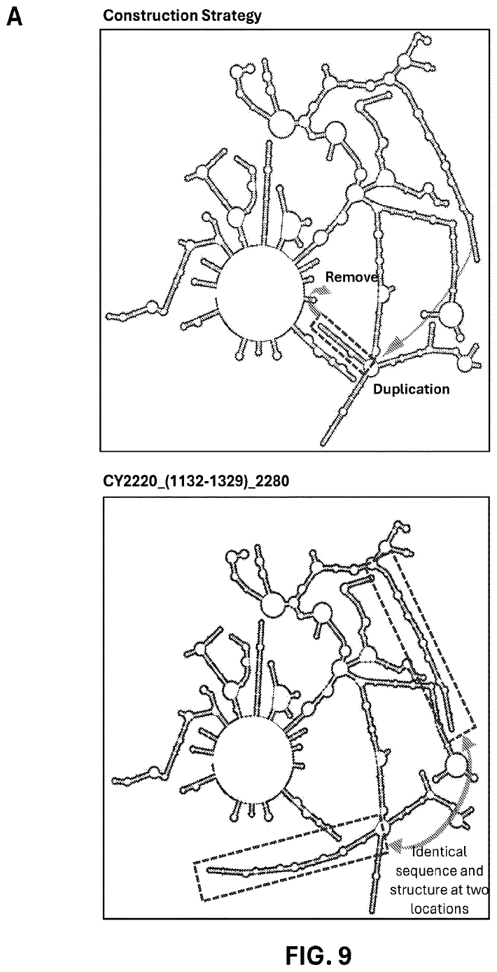

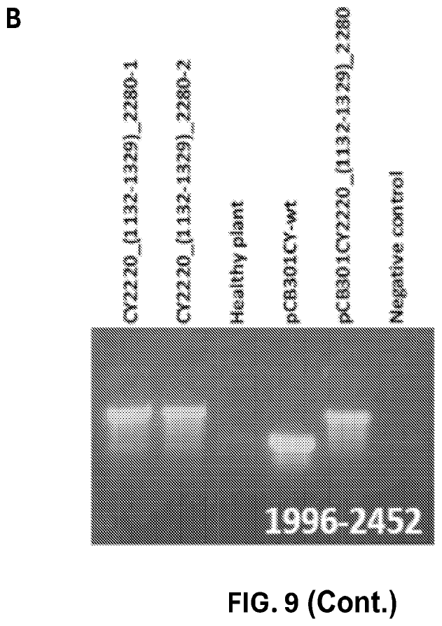

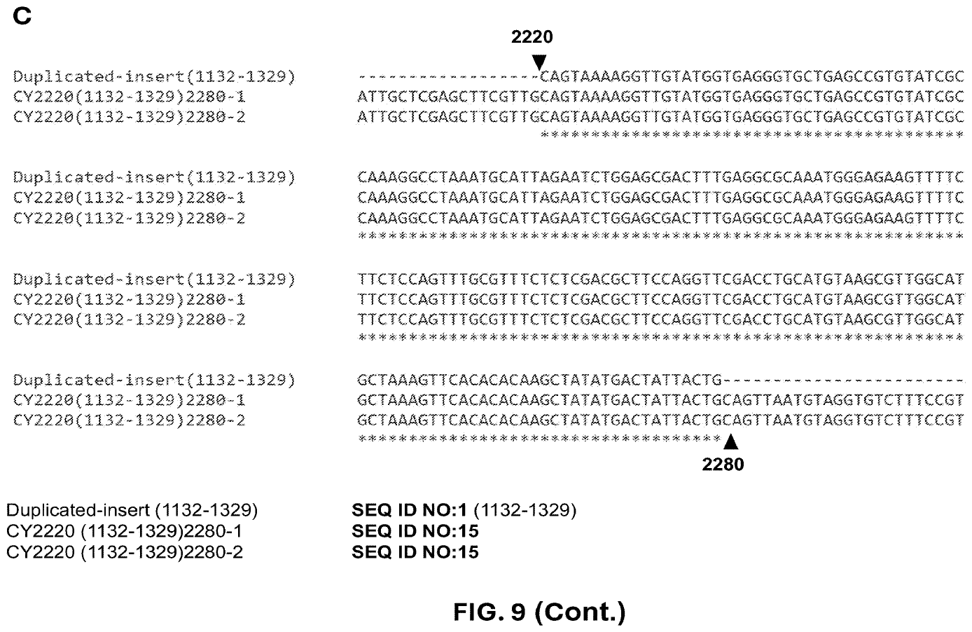

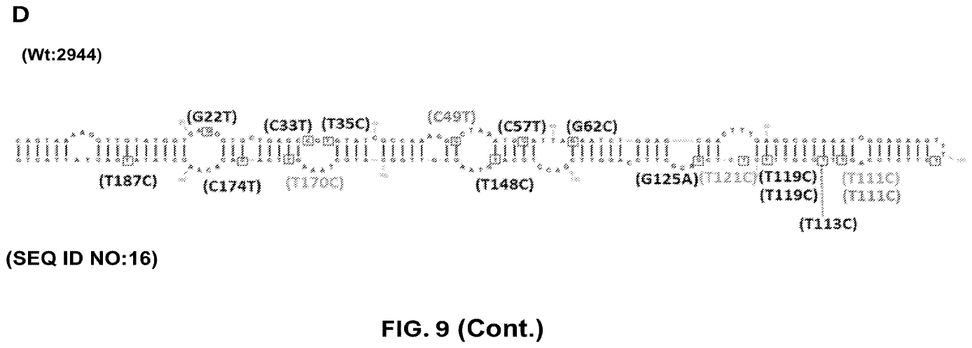

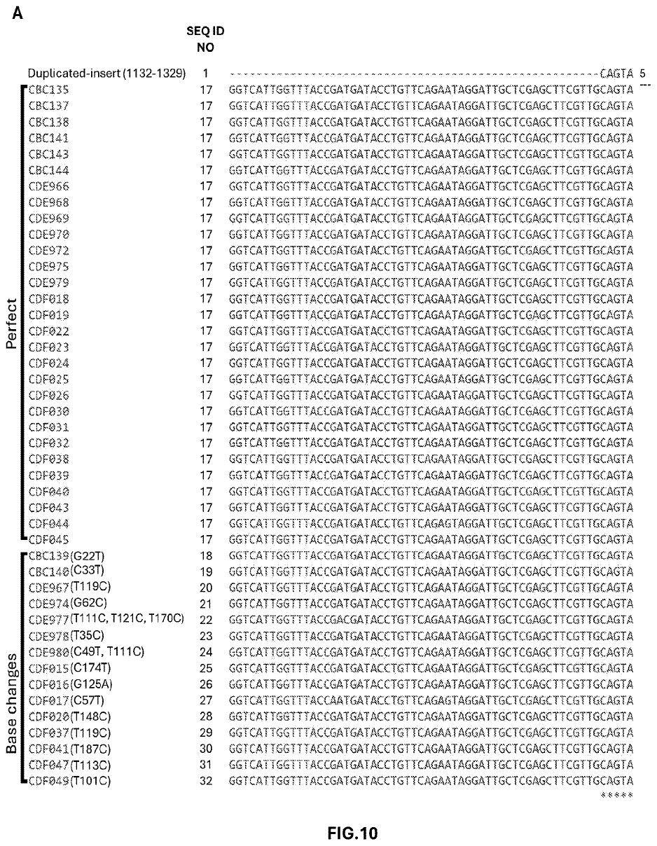

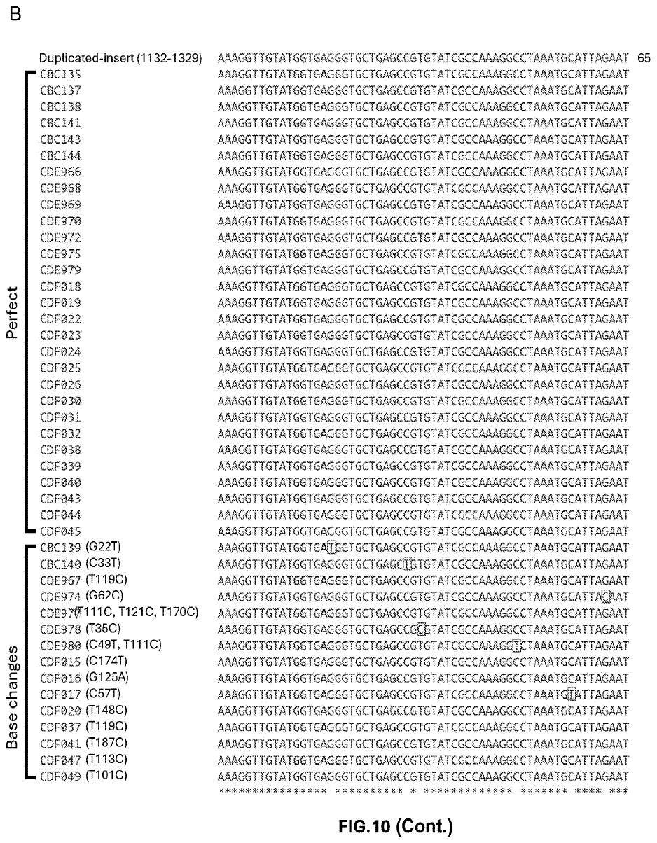

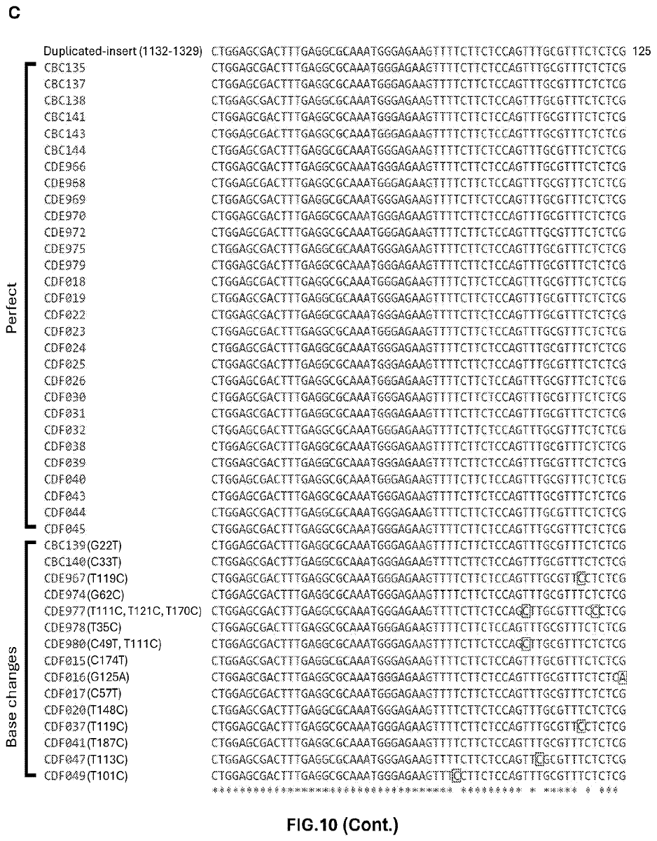

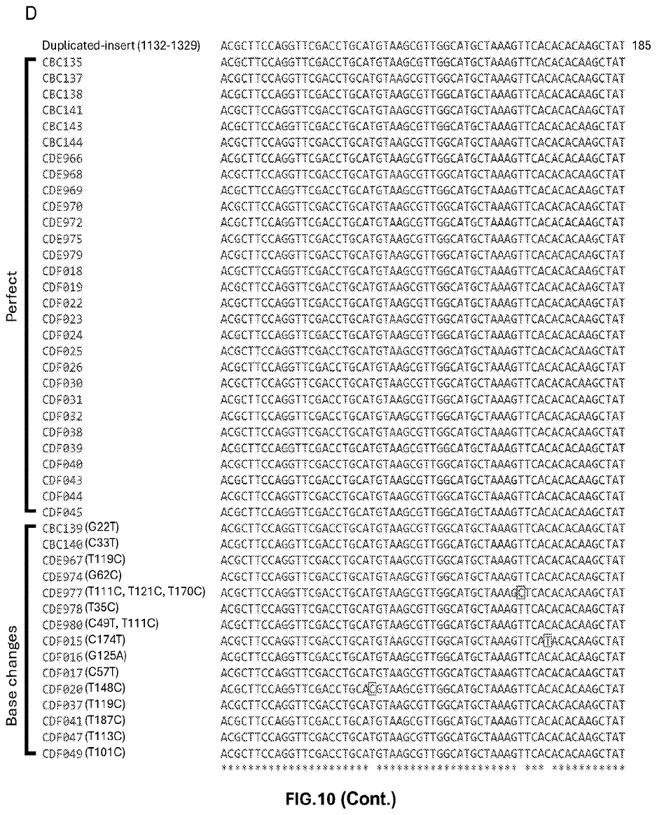

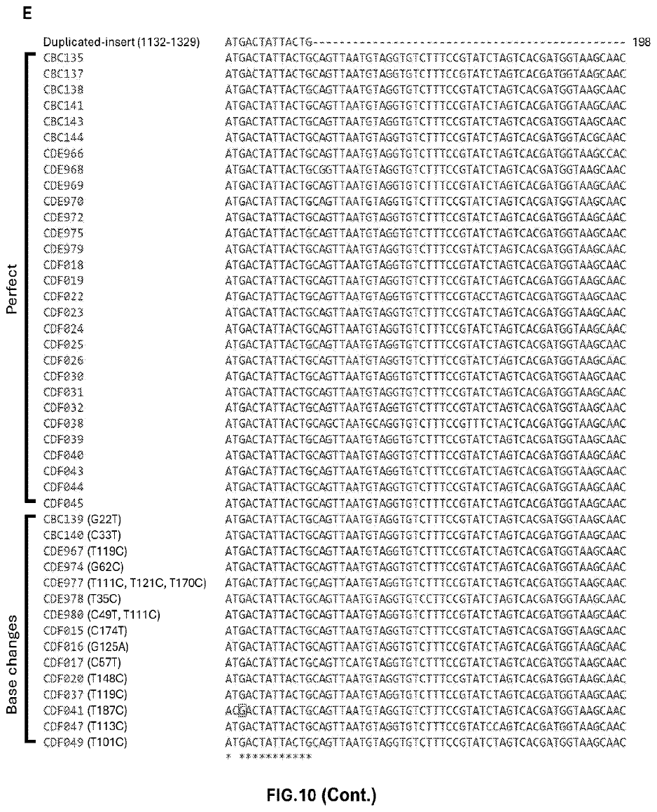

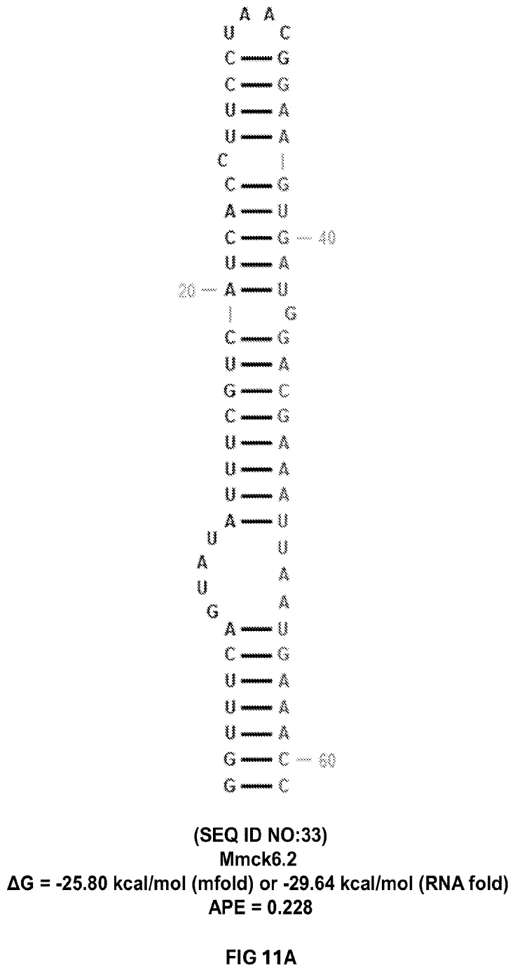

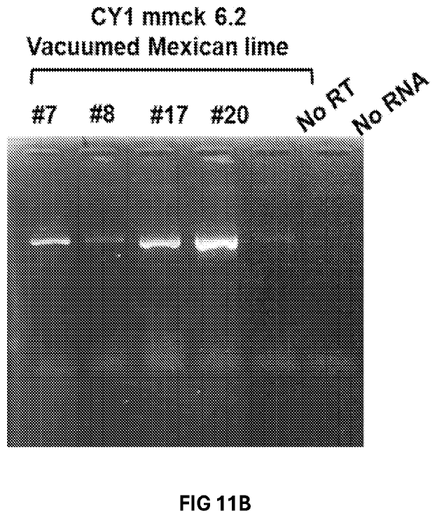

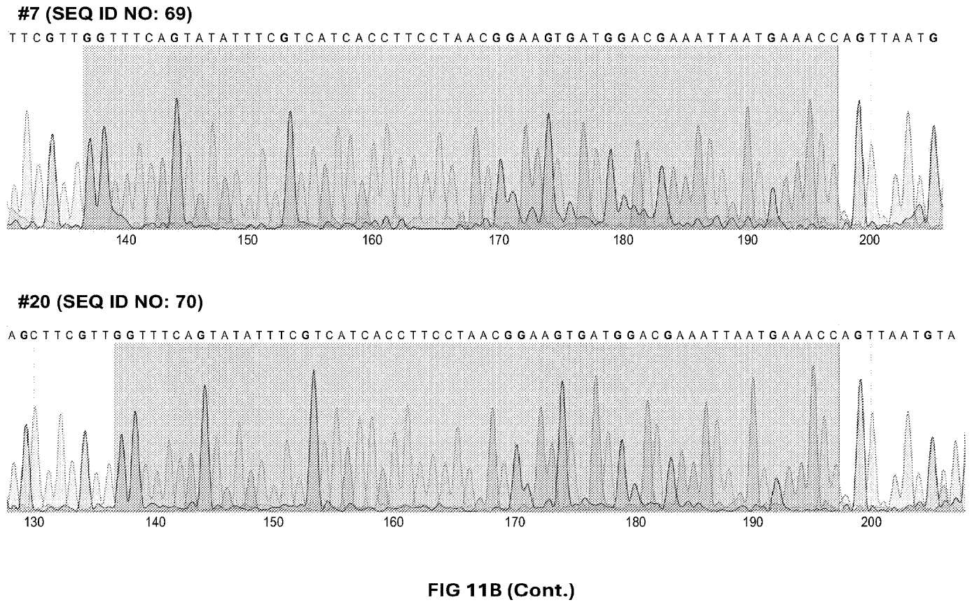

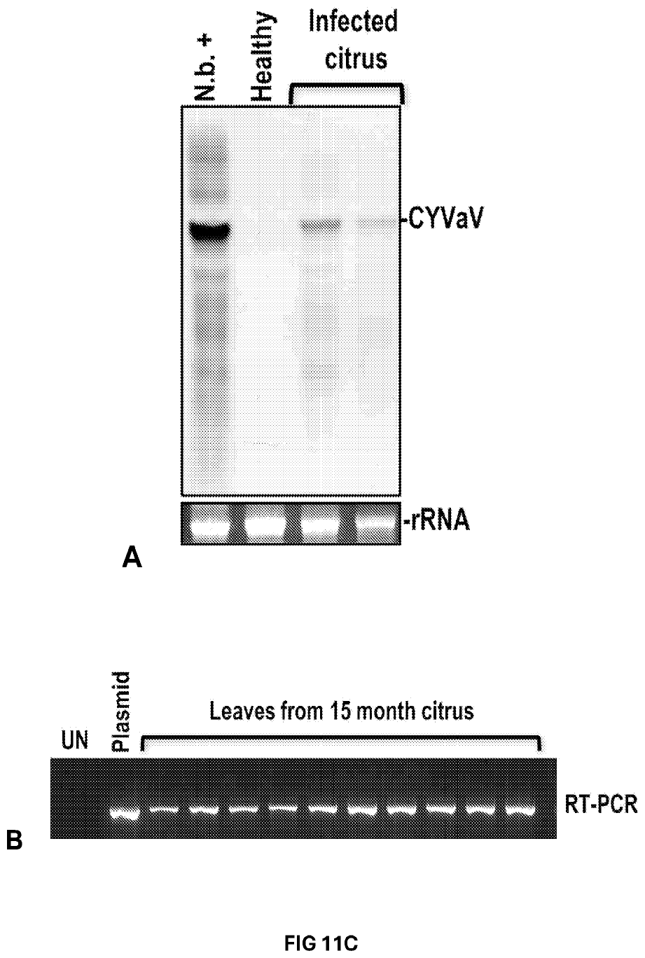

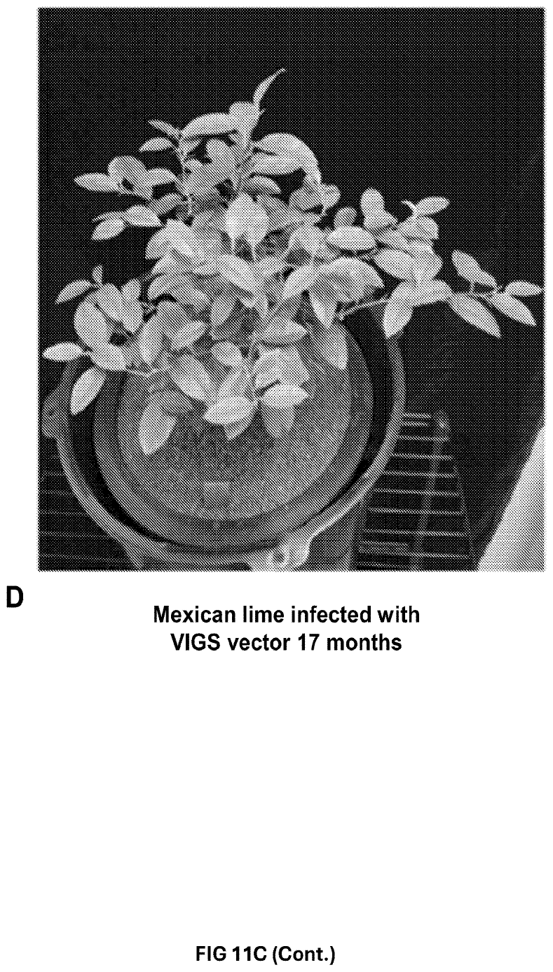

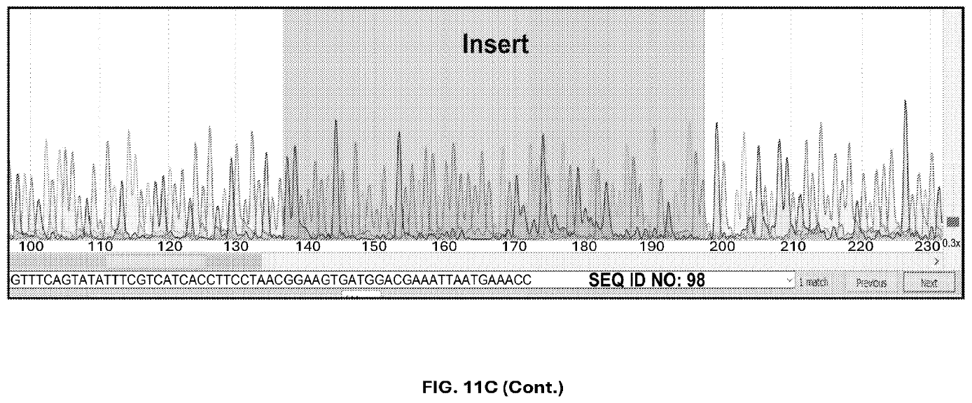

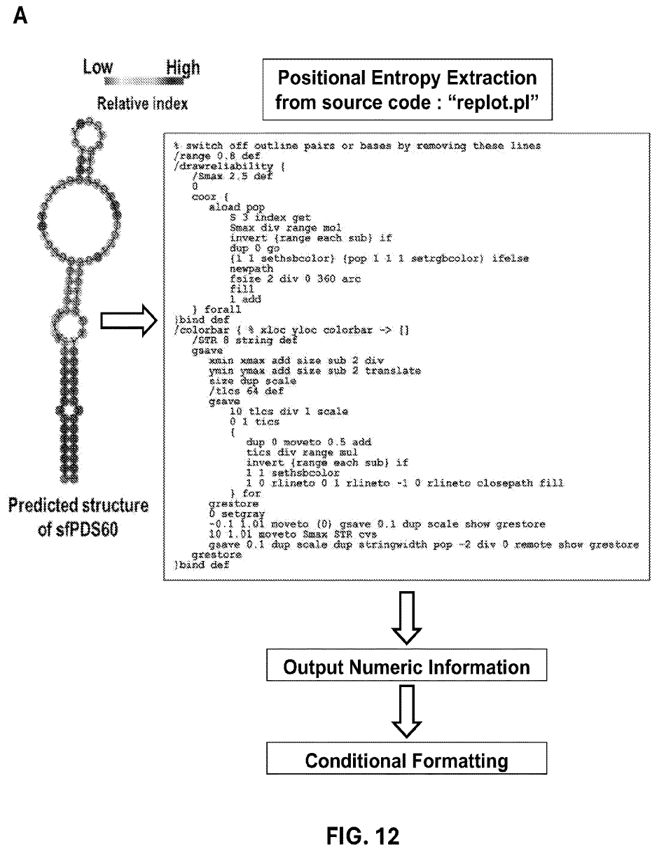

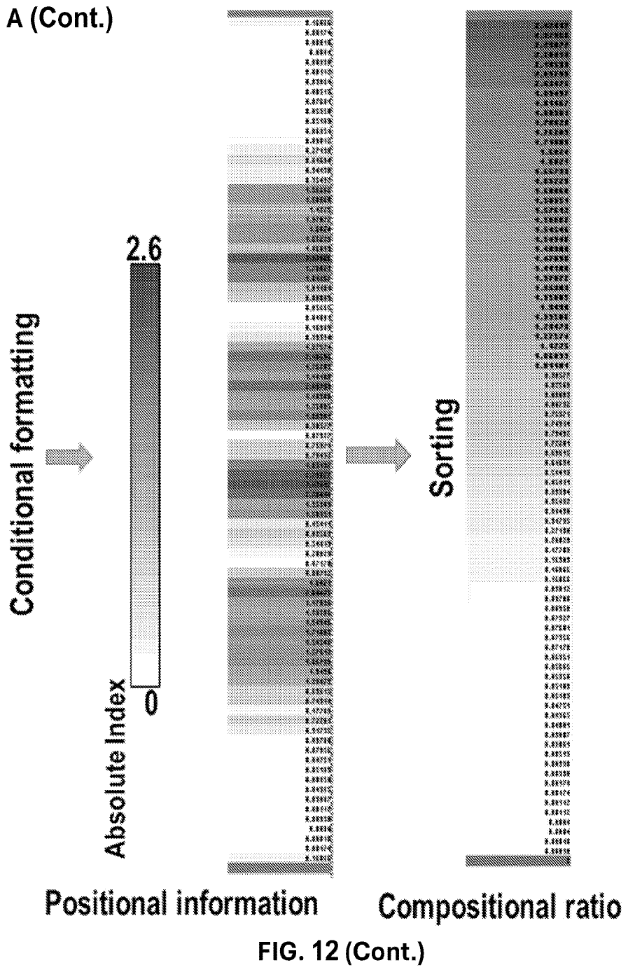

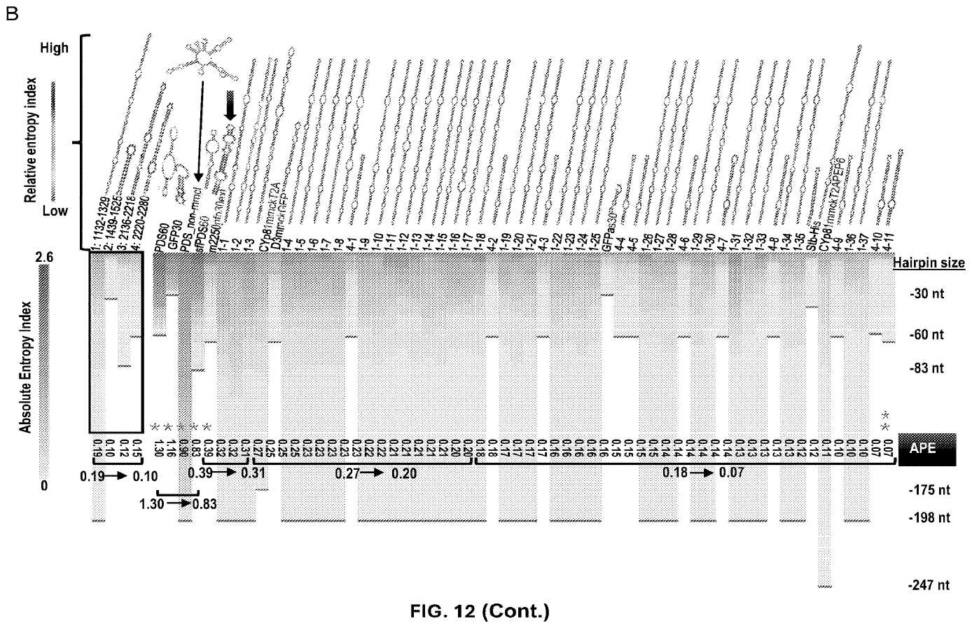

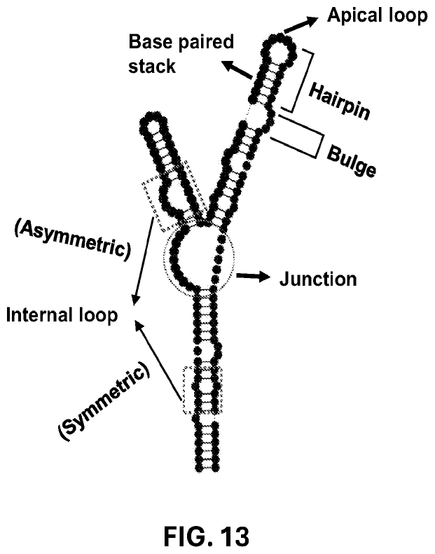

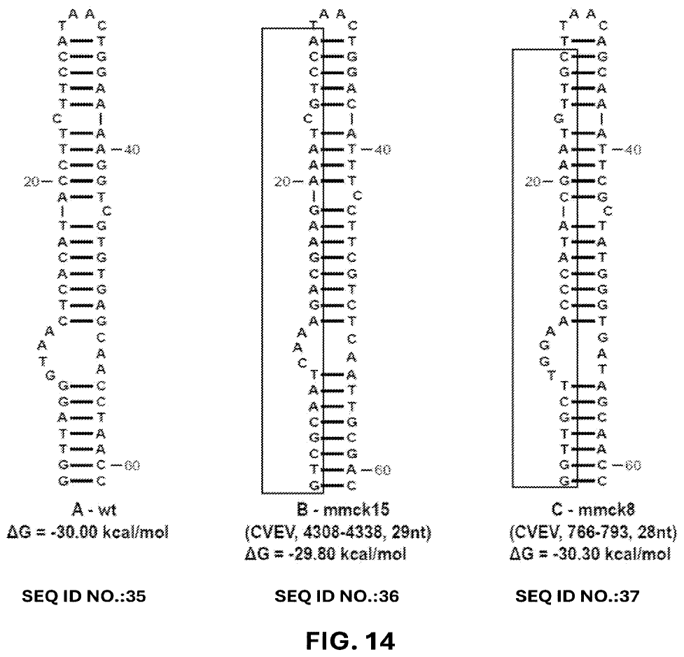

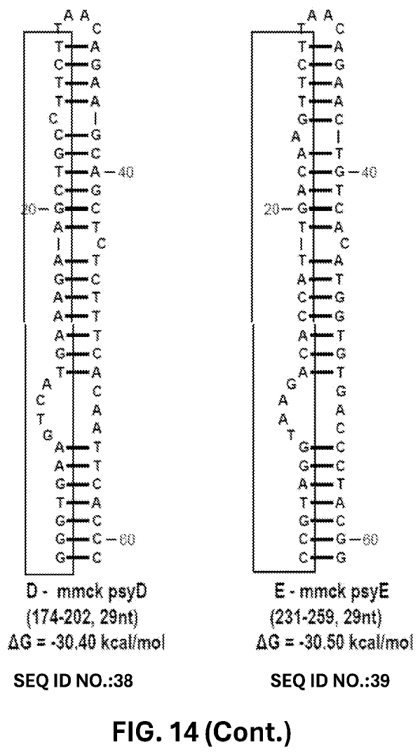

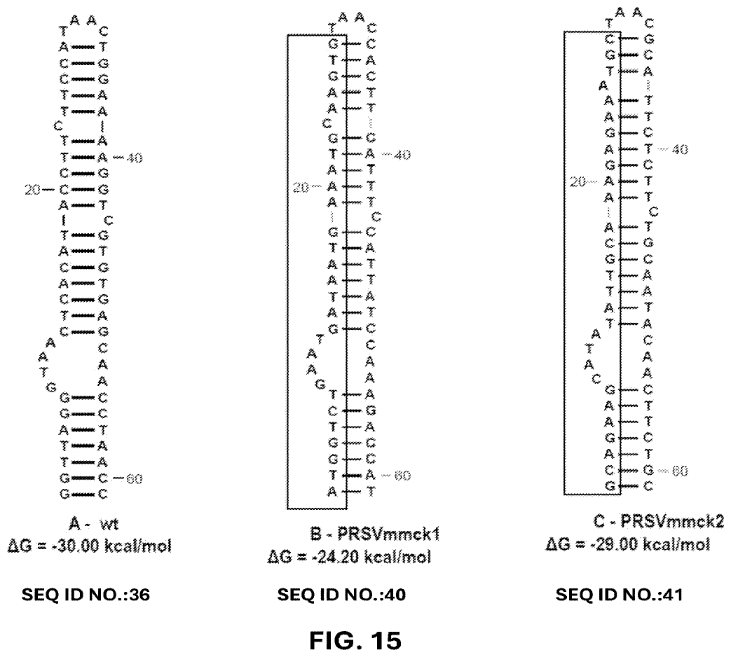

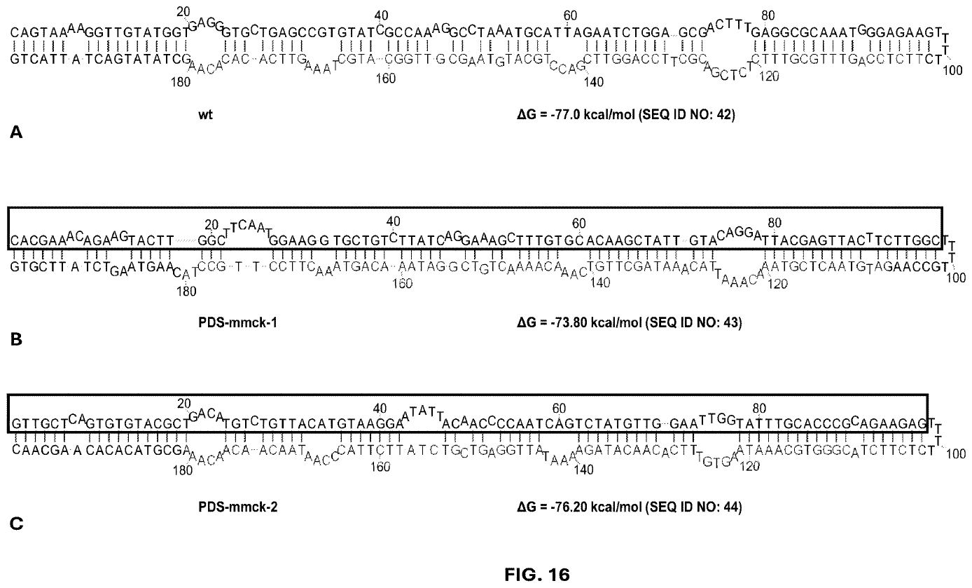

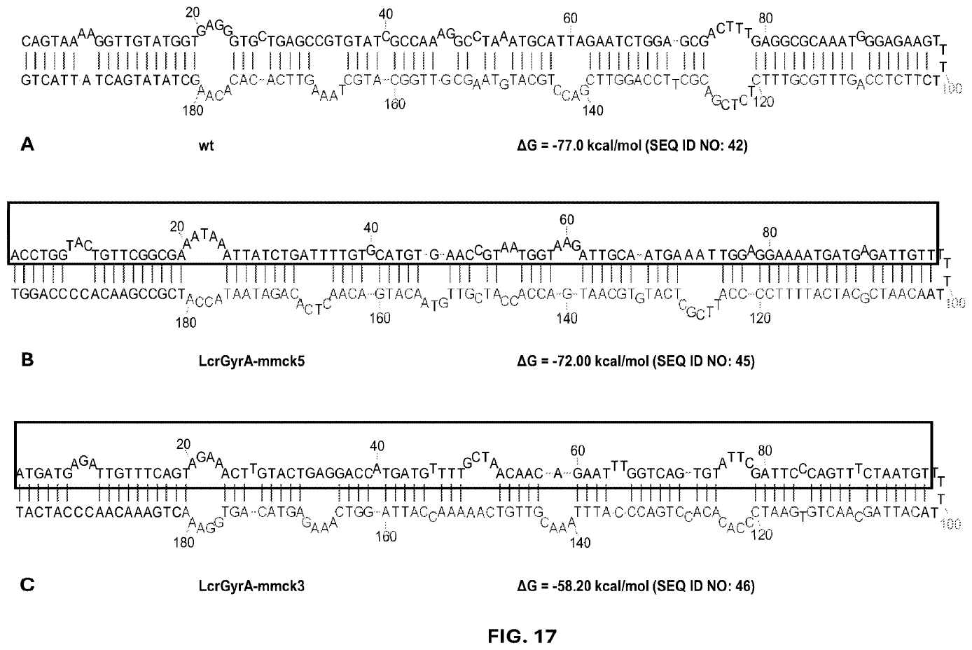

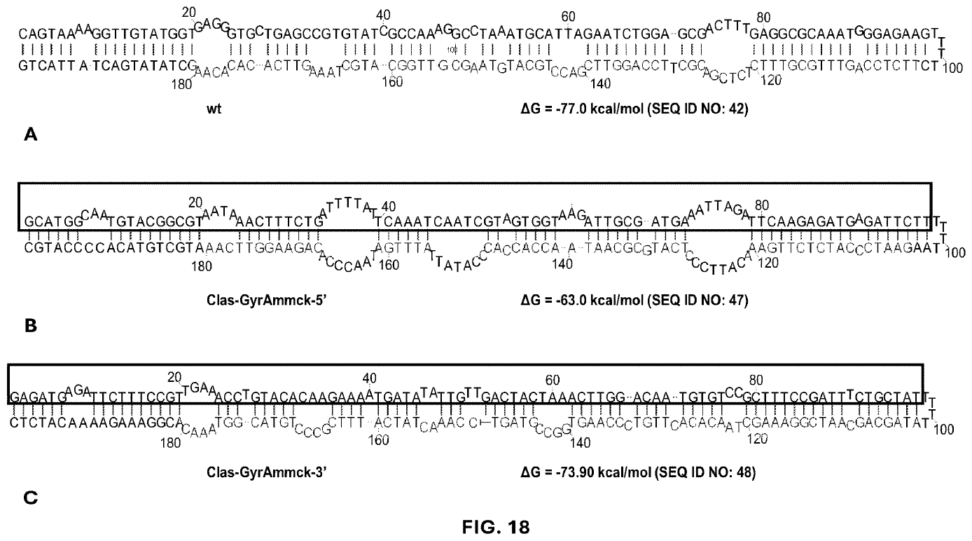

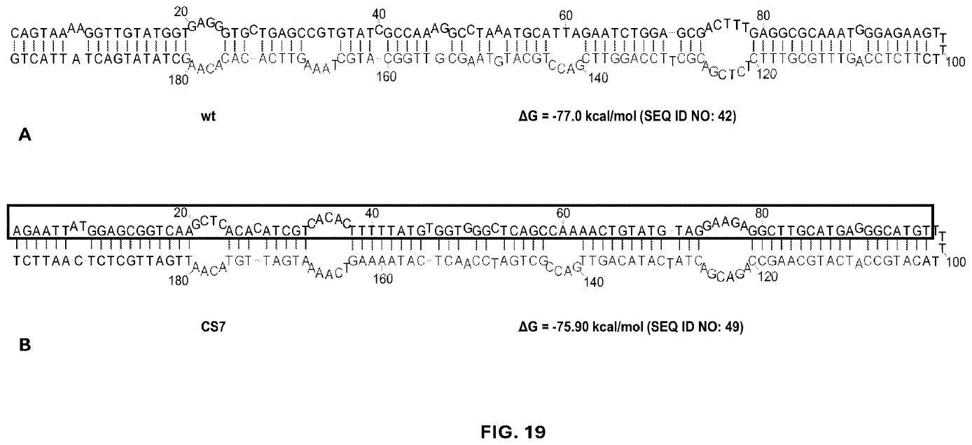

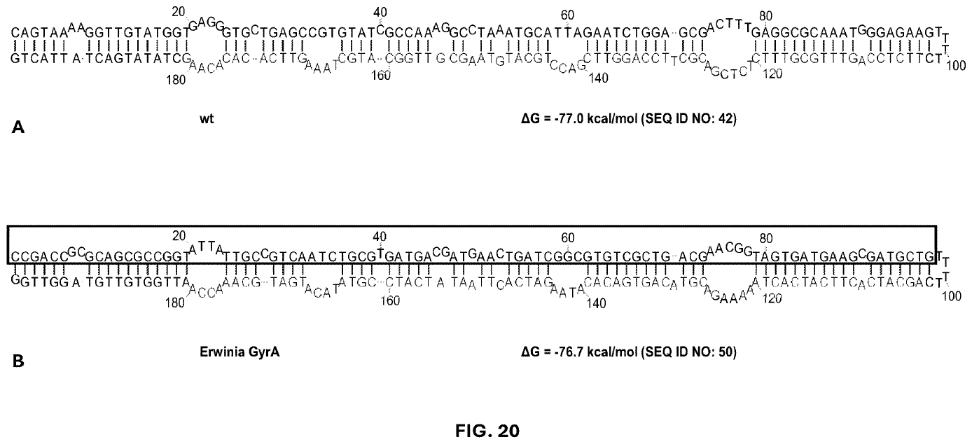

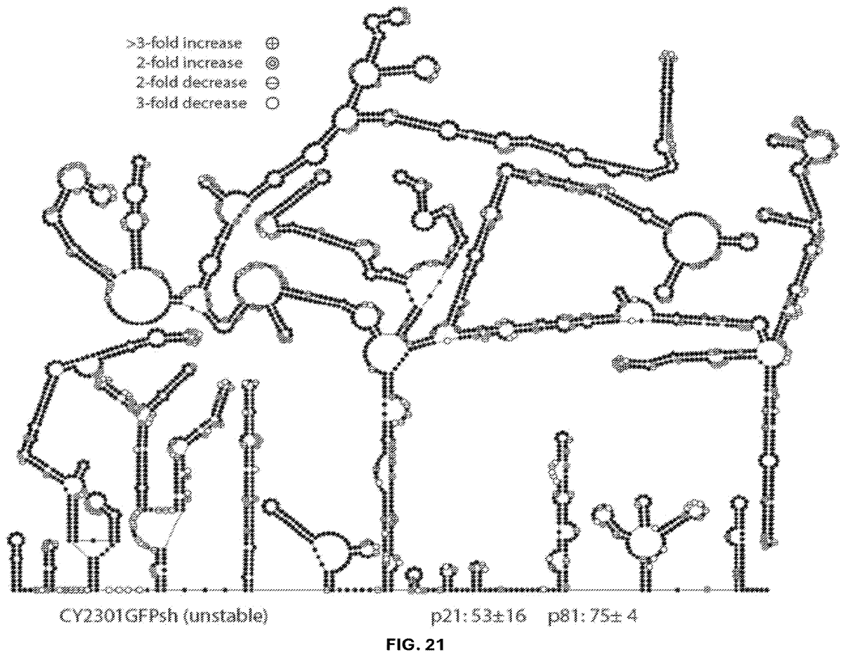

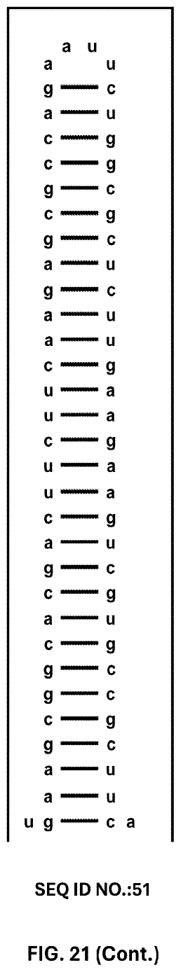

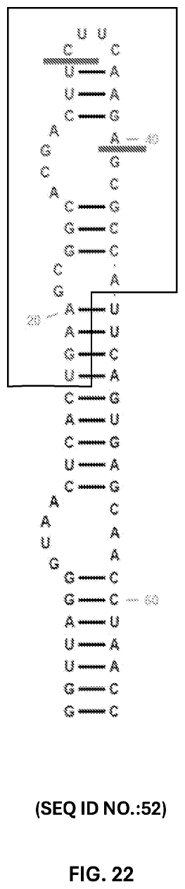

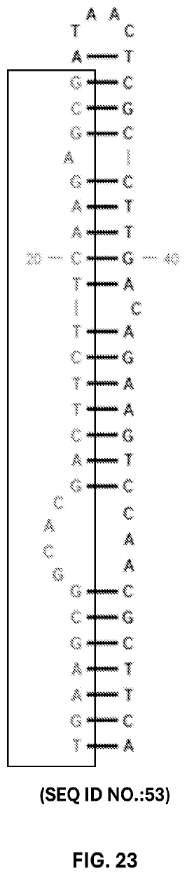

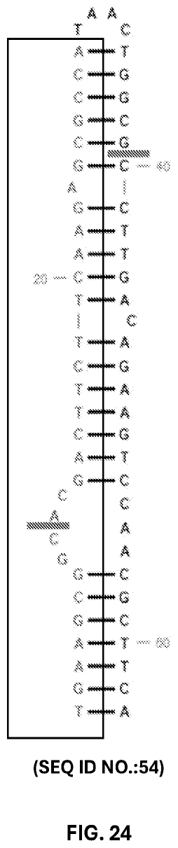

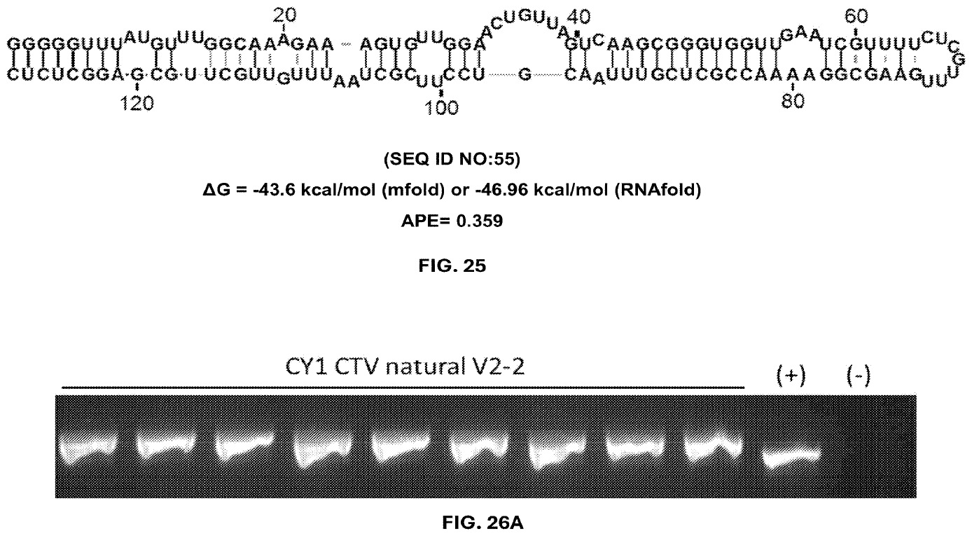

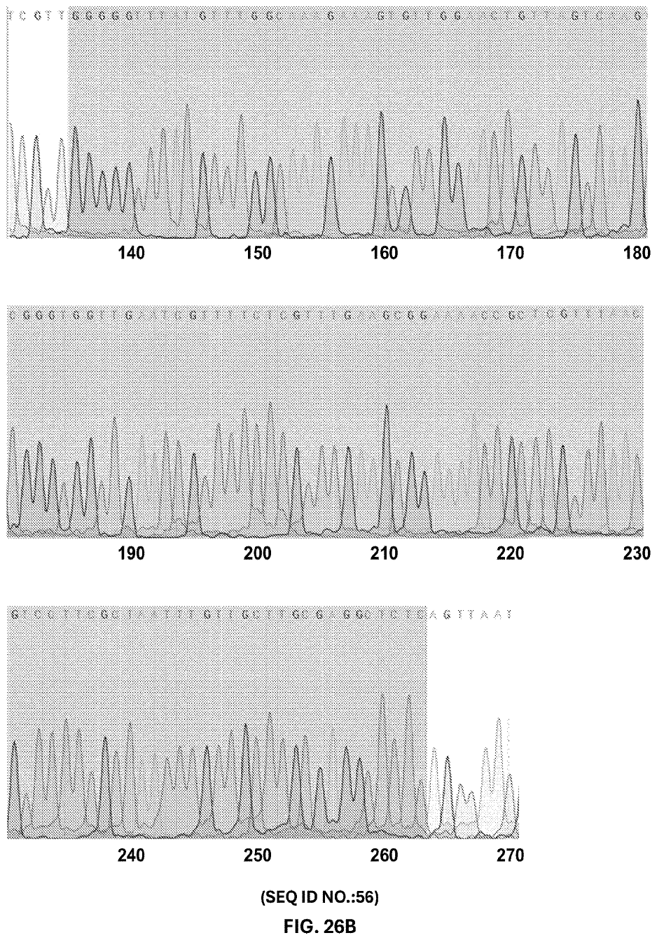

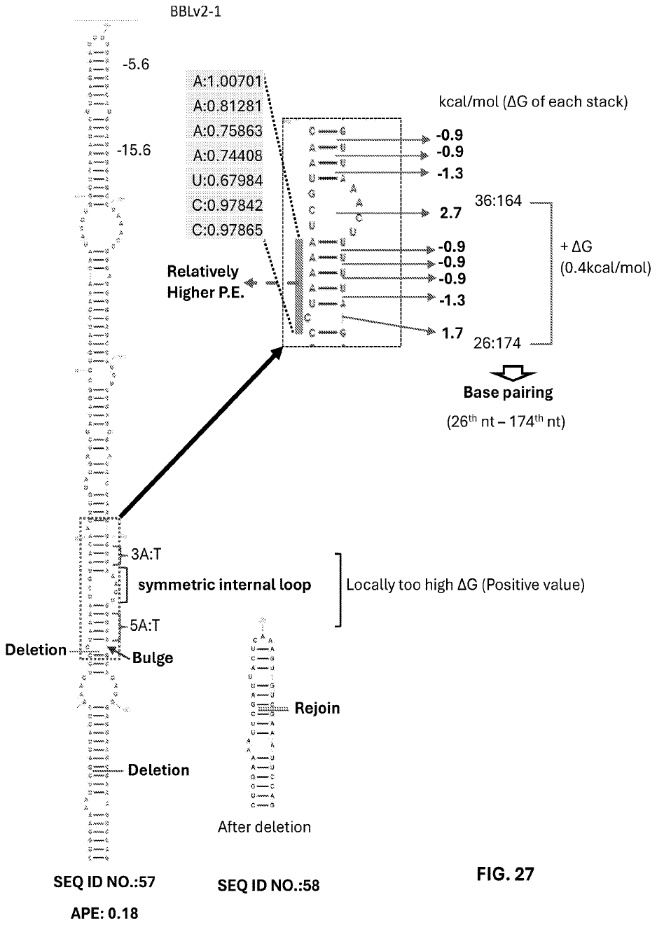

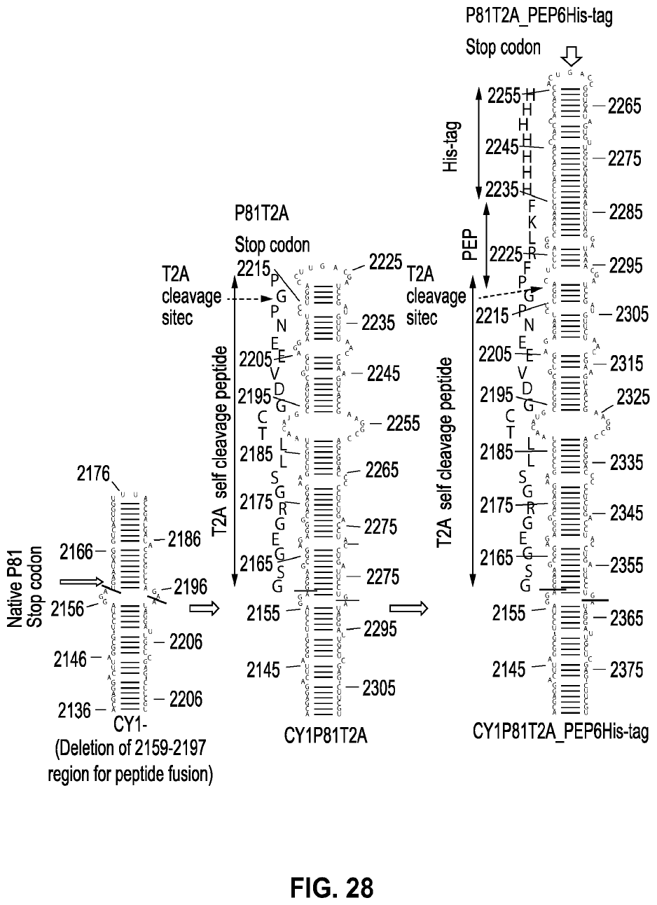

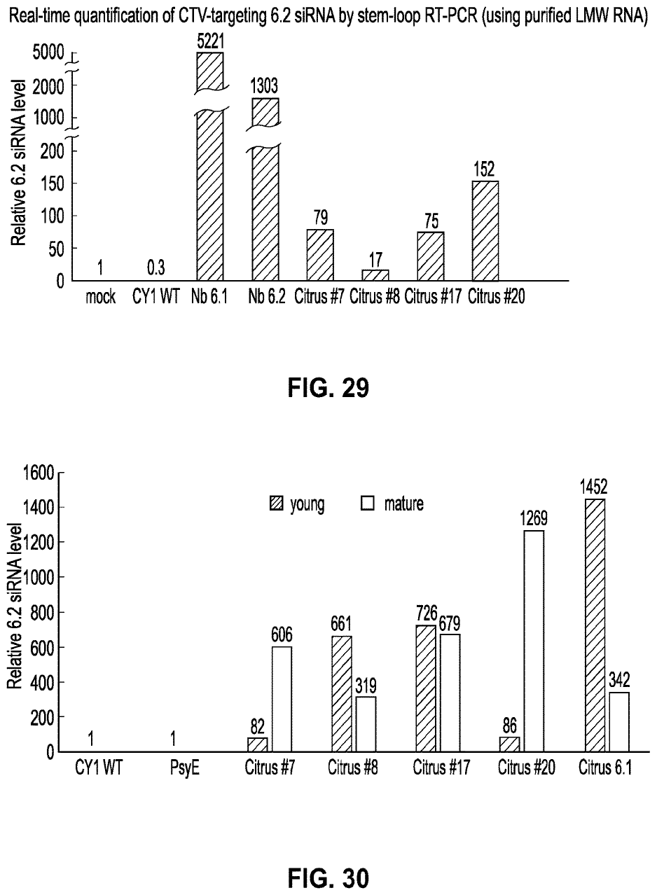

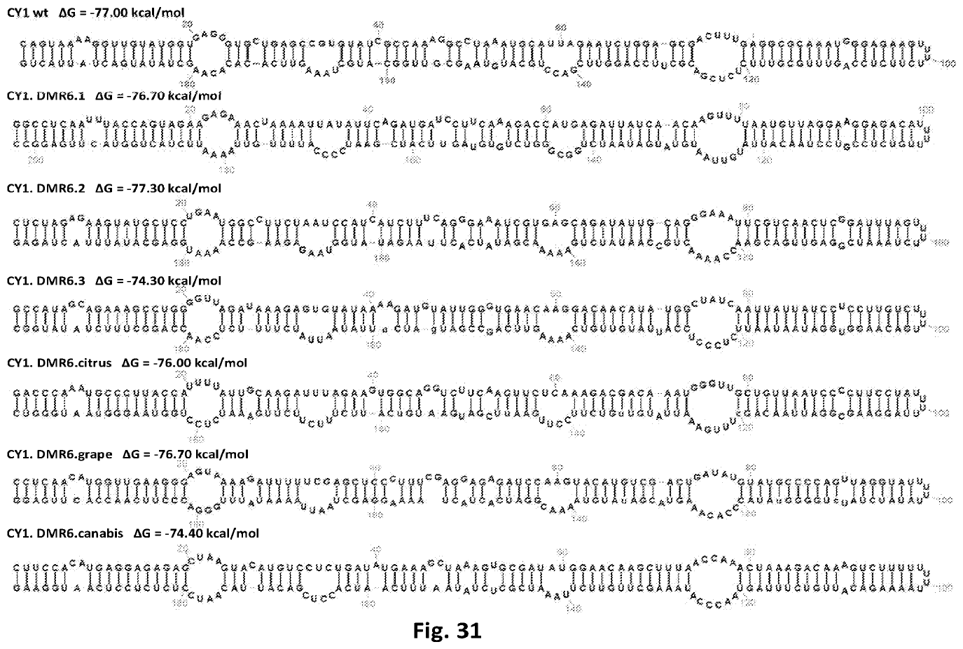

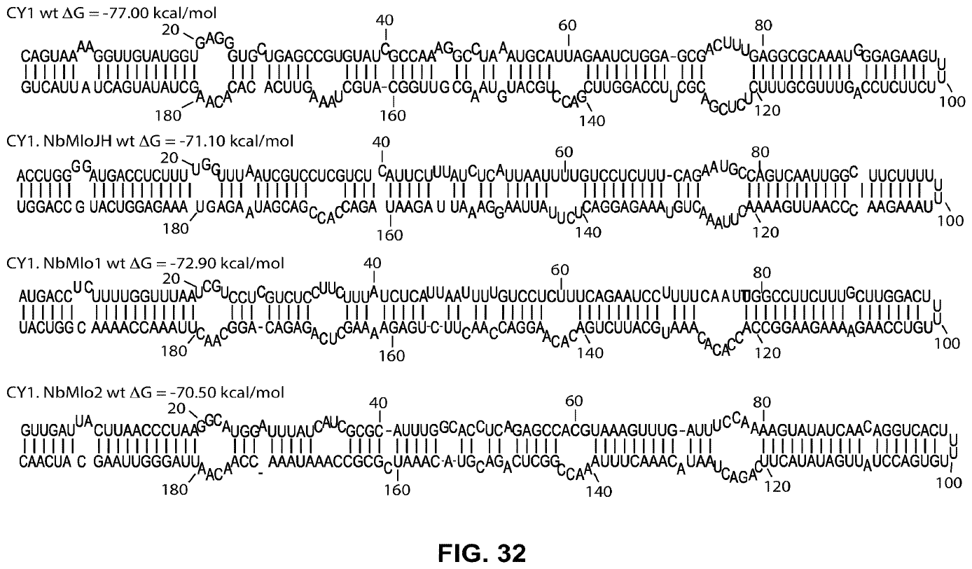

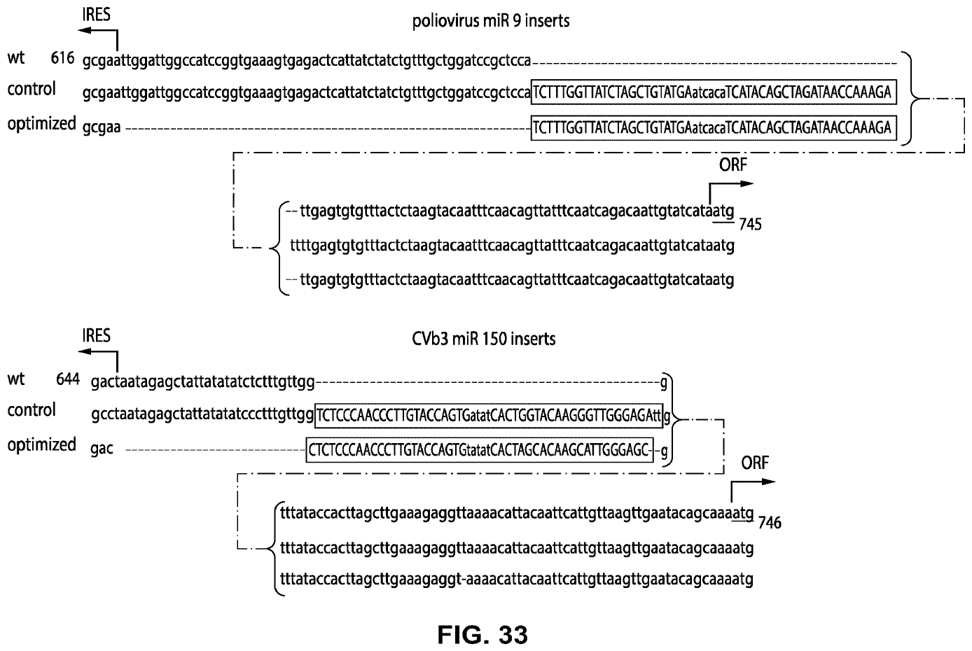

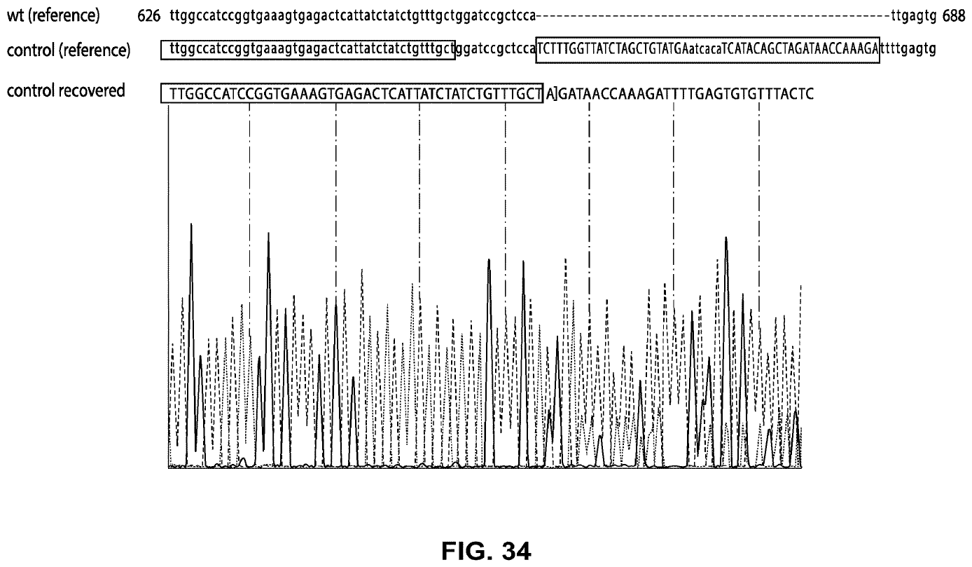

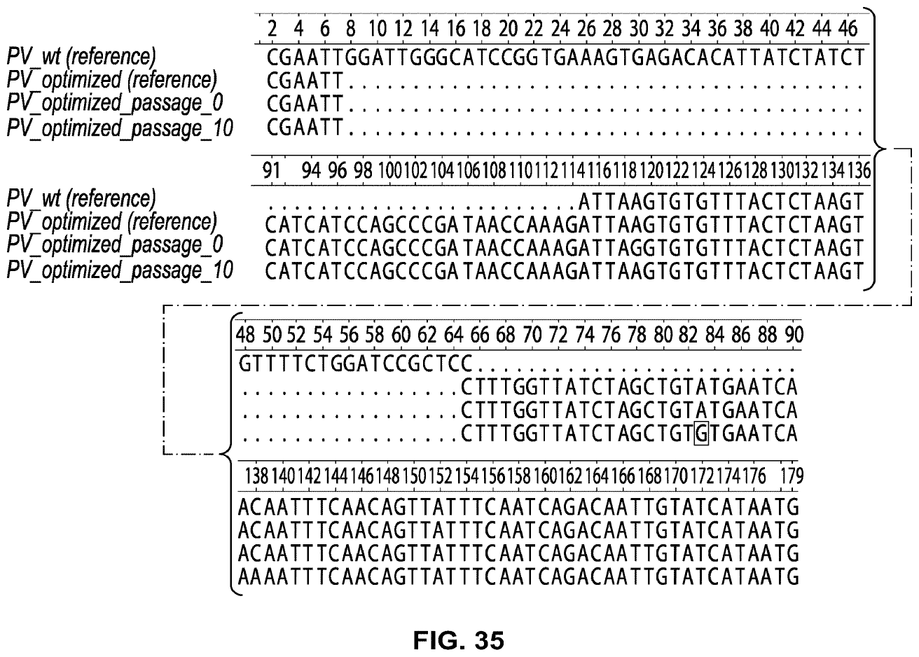

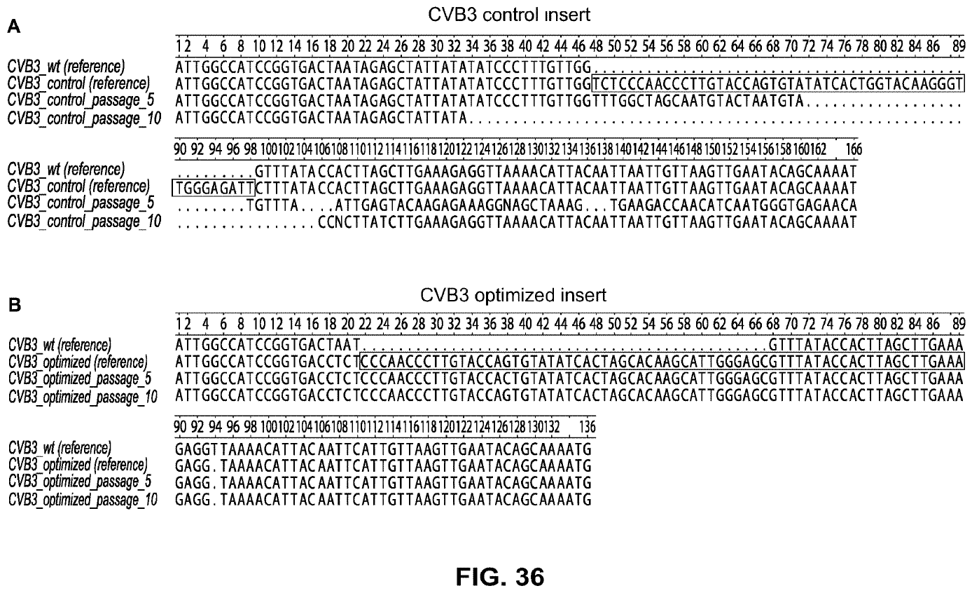

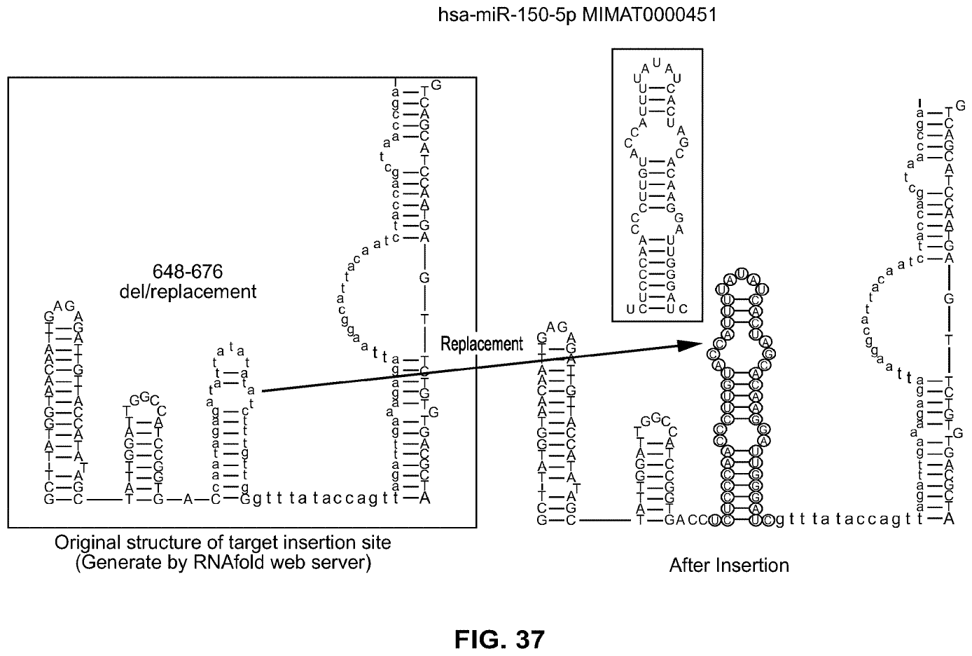

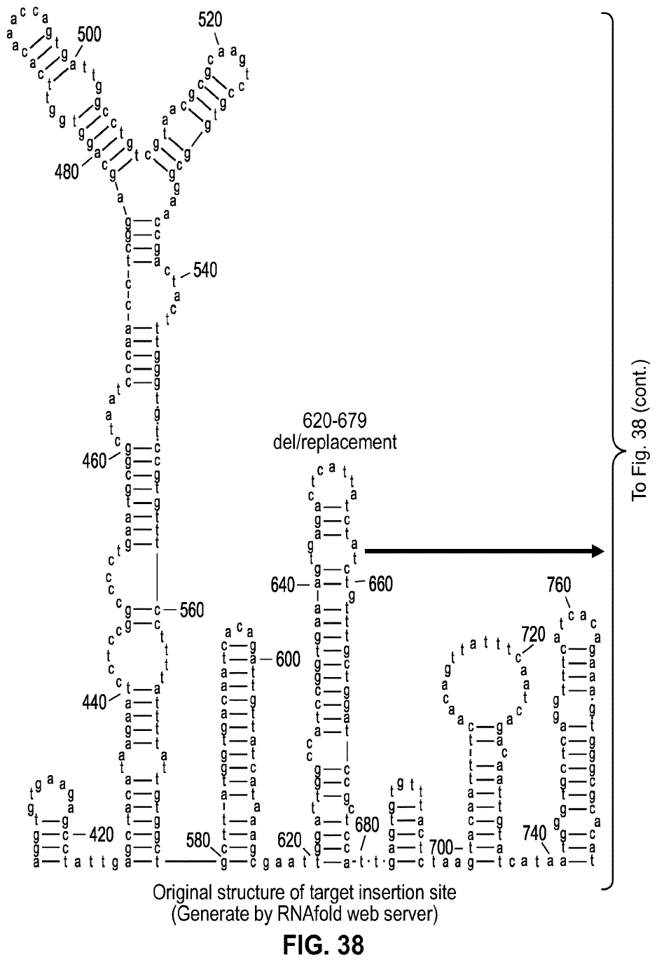

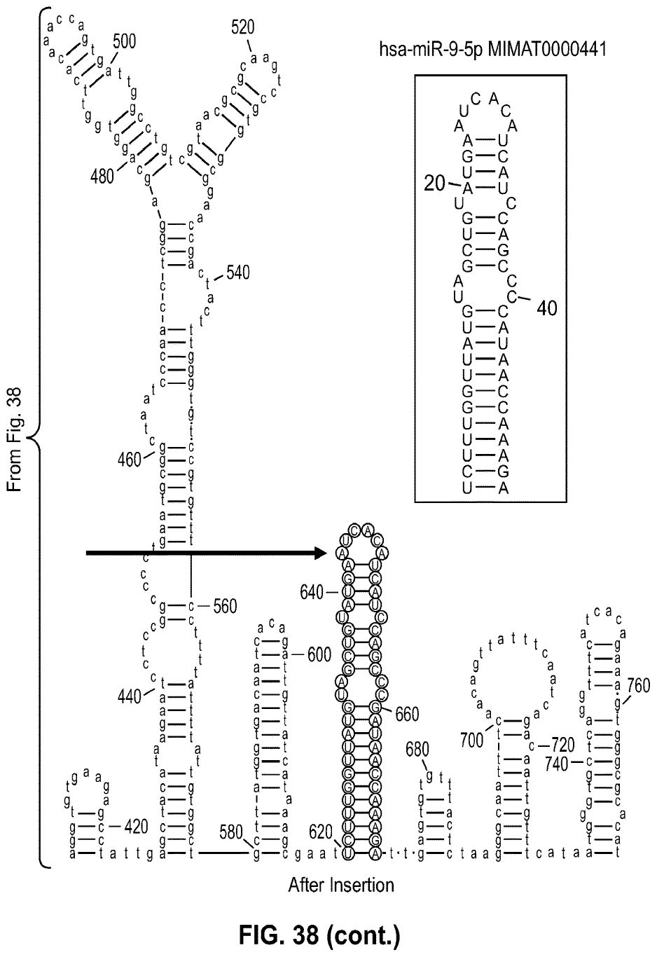

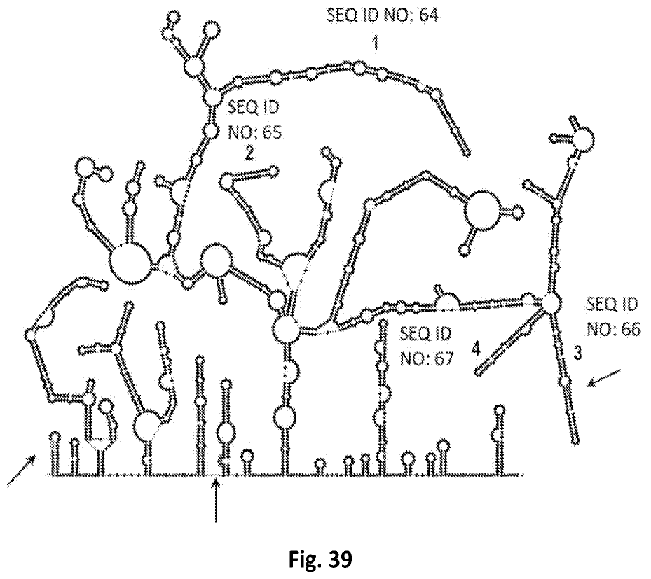

Mimicked hairpins were inserted in one of three different locations, either singly or with a second mimic hairpin. All tested structures were extremely stable, including constructs including two inserts (see FIG. 6). A wide variety of mimicked hairpins have been tested in a variety of locations, including constructs having mimic hairpins based on natural hairpin structures of CY1. In one implementation, relatively large hairpins of CY1 were used (e.g., having a length of about 200 nt or more) in order to stack or incorporate multiple siRNAs into a single insert.

Referring to FIG. 6, the two inserts mimic Structure 4 of CY1. The ΔG of Structure 4 is-30.00 kcal/mol. The APE of Structure 4 is 0.15. The ΔG of insert CS7-V2.2 is-32.50 kcal/mol and the APE is 0.26. The ΔG of insert Mmck6.1 is-28.40 and the APE is 0.15. The sequence of Mmck6.1 is (SEQ ID NO:9):

ggaagugauggacgaaauuaaugauuccauaacuggaacauuacauuucg
ucaacacuucc

The first 24 nt of Mmck6.1, shown in bold above, are an siRNA sequence targeted at CTV. Insert CS7-V2.2 is a targeted against the callose synthase gene of the host plant. The sequence of CS7-V2.2 is (SEQ ID NO:10):

gguaucaaugggcagacgaagauuuggcauaacugccaaucuuccgucug
caaaugauacc

The first 27 nt of CS7-V2.2, shown in bold above, are an siRNA sequence targeted at the callose synthase gene of N. benthamiana.

The (relatively) unstable insert shown in Panel D is CY2301GFP30sh, a conventional (i.e. fully base-paired) hairpin which is also shown in FIG. 5 Panel C and further described by SEQ ID NO:88. Insertion of CY2301GFP30sh was shown to produce SHAPE changes and to effect stability of the RNA structure throughout the CY1 genome (FIG. 61). CY2301GFP30sh was inserted at location 2301 of CY1. After 3-4 weeks of infection, a PCR assay identified a band corresponding to CY1 without the CY2301GFP30sh insert. The batch sequencing of CY2301GFP30sh show in FIG. 6 Panel D shows heterogeneity, further indicating the presence of replicates without the insert. The APE, and positional entropy of individual bases, of CY2301GFP30sh is very low. However, whereas low positional entropy provides stability for inserts with hairpin-like structure, this factor does not predict stability of a conventional (i.e. fully base-paired) insert. The instability of conventional hairpins appears to be related to other considerations.

As demonstrated by the disclosed data, the mimic hairpin inserts are extremely stable within the vector (e.g., a CY1-based vector) and thus are particularly well suited for use as a vector, for example a VIGS vector, for targeting pathogens and endogenous gene expression to control diseases in long lived plants. As known in the art, VIGS is a post-transcriptional gene silencing (PTGS)-based technique that exploits the natural defense mechanisms employed by plants to protect against a viral pathogen. See, e.g., Pantaleo et al. (2007) Molecular Bases of Viral RNA Targeting by Viral Small Interfering RNA-Programmed RISC, J Virol 81 (8): 3797-3806; Ramegowda et al. (2014) Virus-induced gene silencing is a versatile tool for unraveling the functional relevance of multiple abiotic-stress-responsive genes in crop plants, Plant Genetics and Genomics, Vol. 5, Art. 323; Mei et al. (2016) A Foxtail mosaic virus Vector for Virus-Induced Silencing in Maize, Plant Physiol 171:760-772).

Conventional VIGS vectors have not been suitable for long-lived trees. Viruses have relatively limited host ranges, requiring the need to develop a different virus vector for each tree or vine (and sometimes requiring more than one virus vector for the same crop). As such, the cost for the development and approval of a virus that infects a single or limited crop type may be prohibitive. In addition, any virus utilized as a vector should be relatively mild or asymptomatic, thus eliminating numerous viruses as suitable vectors. In addition, sequences inserted into conventional viral vectors are generally unstable, with the insert typically remaining intact only for several days or weeks. However, stability is needed for many years for some hosts such as tree and vine crops.

CY1 and CY1-like molecules are particularly well suited for use as a VIGS vector, or vector having an siRNA or other insert, particularly for use in treating tree and vine pathogens requiring long-term stability. CY 1 exhibits an exceptionally wide host range due to its lack of endogenous movement proteins and use of host movement proteins and little or no detrimental symptoms to the host, may be engineered to include extremely stable inserted sequences, and rarely (if ever) unintentionally spread from plant to plant. In addition, CY1 does not encode any RNA silencing suppressor.

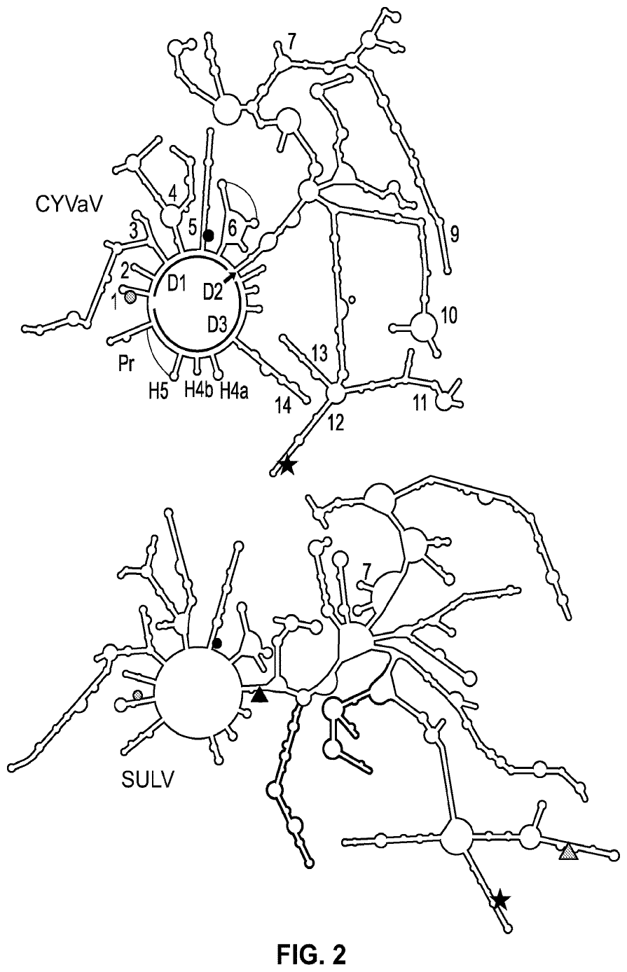

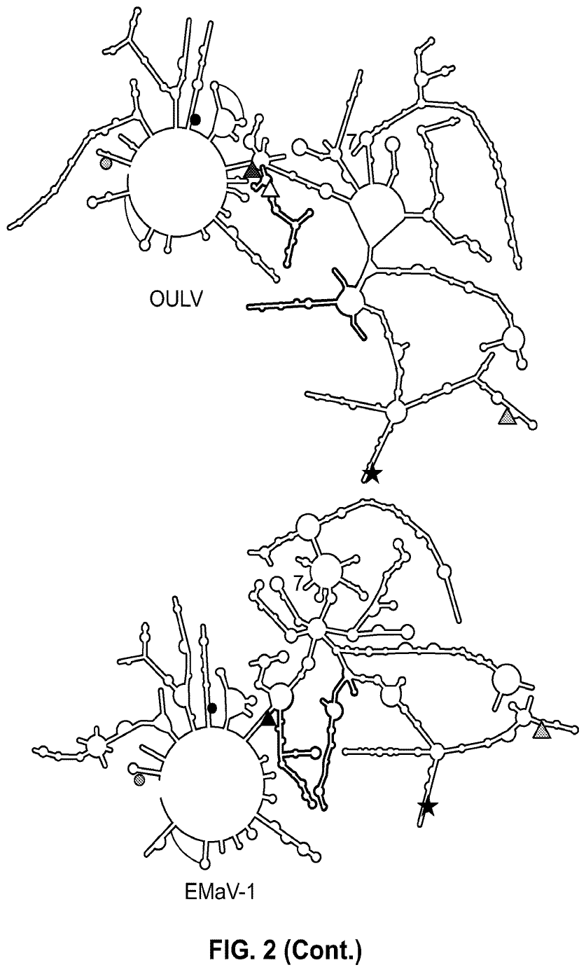

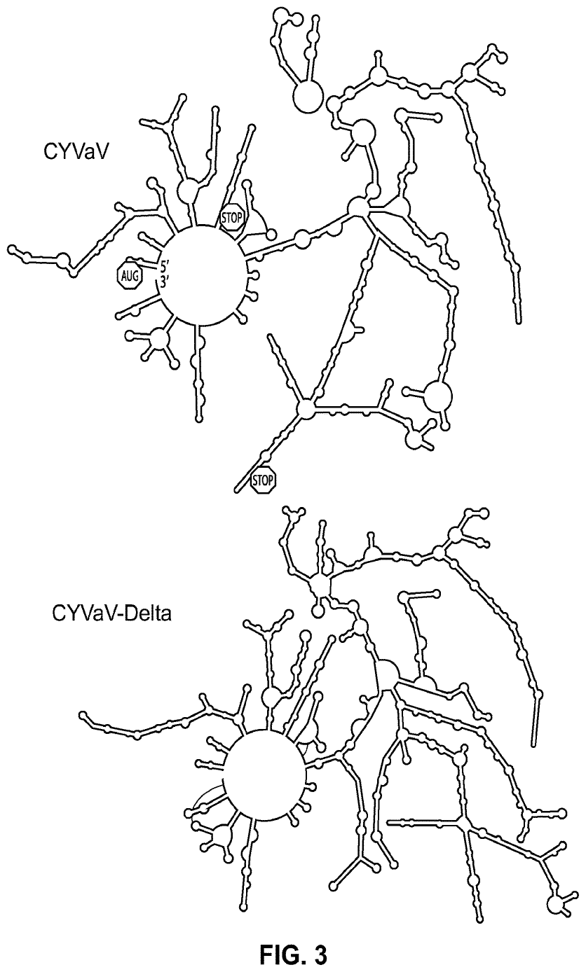

The full length structure of CY1 was determined by SHAPE structure probing and phylogenetic comparisons with the CY1 relatives in Opuntia, Fig and Corn (FIGS. 1C and 2).

### A. Determination of Positional Entropy and Average Positional Entropy

Determining the positional entropy of the bases in an insert starts with determining the secondary structure, i.e. the arrangement of paired and unpaired bases, of the insert. Although RNA folding software may be inaccurate for large sequences, the output of RNA folding software is typically accurate for small sequences, for example inserts of less than 300 nt or less than 200 nt. Additional methods such as SHAPE reactivity may be used to determine the secondary structure of large or difficult inserts, or to confirm the accuracy of or modify a software-produced secondary structure drawing.

Using the RNAfold software as an example, the sequence of an insert is entered into the program. The available results include a minimum free energy (MFE) prediction, an optimal secondary structure of the insert sequence in the MFE state in dot-bracket notation, and a graphical drawing of the dot-bracket notation of the MFE structure. Selecting “EPS” in the download options for the MFE structure drawing encoding positional entropy produces a file with a list of the positional entropy of each base in the insert. The positional entropy of the individual bases are added together and divided by the number of bases to produce the average positional entropy (APE).

Unless stated otherwsie, the values of AG, PE and APE described herein are produced using RNAfold version 2.4.18 available from, or accessed through, the University of Vienna. The program is further described in Mathews D H, Disney M D, Childs J L, Schroeder S J, Zuker M, Turner D H. (2004) Incorporating chemical modification constraints into a dynamic programming algorithm for prediction of RNA secondary structure. Proc Natl Acad Sci USA 101 (19): 7287-92; Gruber A R, Lorenz R, Bernhart S H, Neubock R, Hofacker I L. The Vienna RNA Websuite. Nucleic Acids Research, Volume 36, Issue suppl_2, 1 Jul. 2008, Pages W70-W74, DOI: 10.1093/nar/gkn188; and, Lorenz R, Bernhardt S. H., Honer zu Siederdissen C, Tafer H, Flamm C, Stadler P F and Hofacker I L, “ViennaRNA package 2.0”, Algorithms for Molecular Biology, 6:1 pages 26, 2011.

### B. Stability Assay

Stability of the insert is determined after allowing the insert to replicate in plants for a period of time. The vector is then harvested from the plant and assayed through RT-PCR. A stable insert will show only one band indicative of the intact vector. The appearance of a second band indicates vector replicates that have deleted the insert, and the insert is not entirely stable. In an entirely stable vector, batch sequencing will show no heterogeneity.

### C. Natural Hairpin-like Structures in CYVaV1

FIG. 39 shows the secondary structure of full-length CY1. Each “dot” represents a nucleotide. Numbers denote natural CY1 hairpin-like structures that served as templates for the mimic hairpins. When duplicated and inserted into different locations in the CY1 3′UTR, these hairpin-like structures were stably maintained at all time points assayed until senescence of the host plant.

Structures 1 to 4 in FIG. 39 are identified, based on the position numbers of their nucleotides, as CY1132-1329; CY1493-1525; CY2136-2218 and CY2220-2280 respectively. CY1132-1329 has 6 (3%) of nucleotides with positional entropy of over 1.0 and a standard deviation of positional entropies of 0.30. The average APE is 0.19 and the highest positional entropy (PE) is 1.49. The DG is-87.9 kcal/mol. CY1493-1525 has 1 nucleotide (3%) with positional entropy of over 1.0 and a standard deviation of positional entropies of 0.25. The average APE is 0.10 and the highest PE is 1.00. The DG is-13.1 kcal/mol. CY2136-2218 has 1 (1.2%) of nucleotides with positional entropy of over 1.0 and a standard deviation of positional entropies of 0.25. The average APE is 0.11 and the highest PE is 1.12. The DG is-41.9 kcal/mol. CY2220-2280 has no nucleotides (0%) with positional entropy of over 1.0 and a standard deviation of positional entropies of 0.23. The average APE is 0.15 and the highest PE is 0.80. The DG is-29.9 kcal/mol.

The sequence of CY1132-1329 (structure 1, 198 nucleotides) is (SEQ ID NO:64):

cagtaaaaggttgtatggtgagggtgctgagccgtgtatcgccaaaggc
ctaaatgcattagaatctggagcgactttga

The sequence of CY1493-1525 (structure 2, 33 nucleotides) is (SEQ ID NO:65):

tttatgcttagtgaacttggcattaagcatgaa

The sequence of CY2136-2218 (structure 3, 83 nucleotides) is (SEQ ID NO:66):

acgaggacttaggcgtcctaggatgaatagggtcattggtttaccgatg
atacctgttcagaataggattgctcgagctt

The sequence of CY2220-2280 (structure 4, 61 nucleotides) is (SEQ ID NO:67):

ggttagggtaactcacataccttcttccataactggaaaaggtcgtgtg
agcaacctaacc

In various examples: CY1132-1329 was inserted at 2219/2281 of CYVaV; CY1493-1525 was inserted at 2319/2320 and 2304/2305 of CYVaV (in two separate examples); CY2136-2218 was inserted at 2304/2305 of CYVaV; and, CY2220-2280 was inserted at 2304/2305 of CYVaV. In each case, the resulting vector was stable in planta at the end of a 10-week trial.

A line of best fit through these four hairpin-like structures has the formula ΔG in kcal/mol=−0.44×length of the structure in nt-1.89. When comparing the ΔG of a synthetic construct to the ΔG of a natural hairpin-like structure of similar length, this formula (or a different formula derived from another wild type vector) may be used when a specific natural structure of similar length does not exist. In some examples, stable hairpin-like structures of a selected length are stable when their ΔG is in a range of +/−10 kcal/mol, or −5 to +15 kcal/mol, or +/−20 kcal/mol for inserts of 150 nucleotides or more, of the ΔG determined by the formula for the selected length. A line of best fit through 58 stable synthetic inserts and the four hairpin-like like structures of CY1 has the formula ΔG in kcal/mol=−0.43×length of the structure in nt+1.17. Optionally, hairpin-like structures can be made in a range of −10 to +10 kcal/mol, or −5 to +15 kcal/mol, or −20 to +20− for inserts of 150 nucleoditdes or more, of the ΔG determined by this formula for the selected length.

### D. Example of a Stable Insert Targeted Against CVEV

Two inserts were prepared including siRNA sequences targeted against regions of the CVEV genome. Insert mmck15 (FIG. 14 Panel B) has an siRNA sequence of 29nt complementary to bases 4308-4338 of CVEV. Insert mmck8 (FIG. 14 Panel C) has an siRNA sequence of 28nt complementary to bases 766-793 of CVEV. Sequences are given below. SiRNA portions of the sequences are at the beginning of the sequences and shown in bold type and inside a box in the figure.

The sequence of the wt structure is (SEQ ID NO:35):

ggttagggtaactcacataccttcttccataactggaaaaggtcgtgtg
acaacctaacc

The sequence of mmck15 is (SEQ ID NO:36):

gtcgcaatcaaagacgaagaaatcgtccataactggacatttccttcgt
ctcaattgcgac

The sequence of mmck8 is (SEQ ID NO:37):

ggttgcttggaacccatacgaatgttgcttaacagcaaattcgctatgg
gtgatagcaacc

Wild type structure 4 (FIG. 14 Panel A) has a ΔG of −30.00 kcal/mol and an APE of 0.15. Mmck15 has a ΔG of −29.80 kcal/mol, an APE of 0.18, a standard deviation of PE of 0.28, 1.6% of nucleotides with a PE over 1, and a highest PE of any nucleotide of 1.0. Mmck8 has a ΔG of −30.30 kcal/mol, an APE of 0.16, a standard deviation of PE of 0.24, 1.6% of nucleotides with a PE over 1, and a highest PE of any nucleotide of 1.0. Both mimics were stable after 4 months (the entire duration of the experiment) in N. benthamiana. Secondary structure diagrams of wild type structure 4 (Panel A), mmck 15 (Panel B) and mmck (Panel C) are shown in FIG. 14.

N. benthamiana plants were agroinfiltated with CY1CvEV. After 21 days, the plants were agroinfiltrated with CVEV. After 10 days, the CVEV levels in the CY1CvEV treated plants were 17.6 and 29.5 relative to an untreated control (100).

### E. Further Examples of Inserts Mimicking Structure 4

Referring to FIG. 14, the wild type Structure 4 is shown as Panel A. Panel D is an insert called mmckpsvD with a 29nt siRNA targeting bases 174-202 of peanut stunt virus. Its ΔG is-30.40 kcal/mol and it has an APE of 0.17, a standard deviation of PE of 0.28, 3.3% of nucleotides with a PE over 1, and a highest PE of any nucleotide of 1.0. Panel E is an insert called mmckpsvE with a 29nt siRNA targeting bases 231-259 of peanut stunt virus. Its ΔG is-30.50 kcal/mol and has an APE of 0.12, a standard deviation of PE of 0.25, 1.6% of nucleotides with a PE over 1, and a highest PE of any nucleotide of 1.0. Both mimics were stable after 4 months in N. benthamiana (the entire duration of the experiment). SiRNA portions of the sequences are boxed in the figure.

The sequence of mmckpsvD (SEQ ID NO:38) is shown below. The first 29 nucleotides are the targeting sequence.

gggtgaagtcatgaaagaagctgccttcttaacagaagcagctctcttt
cacaattcaccc

The sequence of mmckpsvE (SEQ ID NO:39) is shown below. The first 29 nucleotides are the targeting sequence.

ccgtaggtaagacaccattgacaagttcttaacagaactgtcacatggt
gtgaccctacgg

Referring to FIG. 15, the wild type Structure 4 is again shown as Panel A. Panel B is an insert called PRSVmmck1 with an siRNA targeting papaya ring spot virus. Its ΔG is-24.20 kcal/mol and has an APE of 0.13, a standard deviation of PE of 0.27, 1.6% of nucleotides with a PE over 1, and a highest PE of any nucleotide of 1.0. Panel C is an insert called PRSVmmck2 with an siRNA targeting papaya ring spot virus. Its ΔG is-29.00 kcal/mol and it has APE of 0.14, a standard deviation of PE of 0.31, 3.3% of nucleotides with a PE over 1, and a highest PE of any nucleotide of 1.6. Both mimics were stable after 4 months in N. benthamiana (the entire duration of the experiment). SiRNA portions of the sequences are boxed in the figure.

The sequence of PRSVmmck1 (SEQ ID NO:40) is shown below. The first 29 nucleotides are the targeting sequence.

atggtctgaatgataatgaaatgcaagtgtaaccacttcatttccatta
tccaaagaccat

The sequence of PRSVmmck2 (SEQ ID NO:41) is shown below. The first 29 nucleotides are the targeting sequence.

gcagaagcatatattgcaaagagaaatgctaacgcattctcttctgcaa
tacaacttctgc

### F. Examples of Inserts Mimicking Structure 1

FIGS. 16-20 each show the wild type Structure 1 found at 1132-1329 of CY1 in Panel A. Its ΔG is-87.9 kcal/mol and its APE is 0.19.

FIG. 16 shows PDS-mmck-1 (Panel B) and PDS-mmck-2 (Panel C). These inserts target phytoene desaturase (PDS). PDS is a widely used reporter gene to demonstrate silencing an endogenouse gene in plants. Silencing the PDS gene produces a photobleached phenotype. ΔG of PDS-mmck-1 is-81.80 kcal/mol and has an APE of 0.16, a standard deviation of PE of 0.27, 3.0% of nucleotides with a PE over 1, and a highest PE of any nucleotide of 1.6. ΔG of PDS-mmck-2 is-82.2 kcal/mol and it has an APE of 0.21, a standard deviation of PE of 0.32, 4.0% of nucleotides with a PE over 1, and a highest PE of any nucleotide of 1.5. SiRNA portions of the sequences are boxed in the figure.

The sequence of the wt structure is (SEQ ID NO:42):

cagtaaaaggttgtatggtgagggtgcttgagccgtgtatcgccaaagg
cctaaatgcattagaatctggagcgactttgaggcgcaaatgggagaag
ttttcttctccagtttgcgtttctctcgacgcttccaggttcgacctgc
atgtaagcgttggcatgctaaagttcacacacaagctatatgactatta
ctg

The sequence of the PDS-198mmck-1 insert (SEQ ID NO:43) is shown below (target sequence shown in bold):

cacgaaacagaagtacttggcttcaatggaaggtgctgtcttatcagga
aagctttgtgcacaagctattgtacaggattacgagttacttcttggct
tttgccaagatgtaactcgtaaacaaattacaaatagcttgtcaaacaa
aactgtcggataaacagtaaacttccttgcctacaagtaagtctattcg
tg

The sequence of the PDS-198mmck-2 insert (SEQ ID NO:44) is shown below (target sequence shown in bold):

gttgctcagtgtgtacgctgacatgtctgttacatgtaaggaatattac
aaccccaatcagtctatgttggaattggtatttgcacccgcagaagagt
tttctcttctacgggtgcaaataagtgtttcacaacatagaaaatattg
gagtcgtctattcttacccaataacaacaacaaagcgtacacacaagca
ac

FIG. 17 shows LcrGyrA-mmck5 (Panel B) and LcrGyr-mmck3 (Panel C). These inserts target the GyrA gene of Liberibacter crescens. ΔG of LcrGyrA-mmck5 is-82.5 kcal/mol and it has an APE of 0.32, a standard deviation of PE of 0.40, 6.1% of nucleotides with a PE over 1, and a highest PE of any nucleotide of 1.9. ΔG of LcrGyrA-mmck3 is-69.4 kcal/mol (by RNAfold) or −58.20 kcal/mol (by mfold*) and it has an APE of 0.32, a standard deviation of PE of 0.47, 13.6% of nucleotides with a PE over 1, and a highest PE of any nucleotide of 1.9. LcrGyrA-mmck3 is unusual in that its MFE when calculated by two different programs (mfold and RNAfold) varies, whereas for most hairpin-like structures the MFE calculaed by different programs is similar. Values described herein are from RNAfold unles stated otherwise. LcrGyrA-mmck3 also had a higher standard deviation of PE and more high PE bases than most other haipin-like structures tested. Further, LcrGyrA-mmck3 has an MFE that is 19.6 kcal/mol greater than the MFE predicted by the formula (−0.44 multiplied by the length of the insert (# of nt)−1.89) and 18.5 kcal/mol greater than the wildtype CY1 hiarpin-like region of the same length (198 nucleotides). LcrGyrA-mmck3 suggests that a larger positive variance in MFE may be acceptable for larger inserts, for example insets of 150 nucleotides or more. It is not known whether a difference in stability between, for example, LcrGyrA-mmck3 and LcrGyrA-mmck5 may have emerged after a longer experiment. SiRNA portions of the sequences are boxed in the figure.

The sequence of the LcrGyrA-mmck5 insert (SEQ ID NO:45) is shown below (target sequence shown in bold):

acctggtactgttcggcgaaataaattatctgattttgtgcatgtgaac
cgtaatggtaagattgcaatgaaattggaggaaaatgatgagattgttt
tttaacaatcgcatcattttccccattcgctcatgtgcaatgaccacca
tcgttgtaacatgacaactcacagataataccatcgccgaacaccccag
gt

The sequence of the LcrGyrA-mmck3 insert (SEQ ID NO:46) is shown below (target sequence shown in bold):

atgatgagattgtttcagtagaaacttgtactgaggaccatgatgtttt
gctaacaacagaatttggtcagtgtattcgattcccagtttctaatgtt
tttacattagcaactgtgaatcccacacacctgacccatttaaacgttg
tcaaaaaccattaggtcaaagagtacagtggaaactgaaacaacccatc
at

FIG. 18 shows Clas-GyrAmmck-5′ (Panel B) and Clas-GyrAmmck-3′ (Panel C). These inserts target the GyrA gene of Liberibacter asiaticus. ΔG of Clas-GyrAmmck-5′ is-75.3 kcal/mol and it has an APE of 0.23, a standard deviation of PE of 0.36, 5.1% of nucleotides with a PE over 1, and a highest PE of any nucleotide of 1.2. ΔG of Clas-GyrAmmck-3′ is-87.3 kcal/mol and it has an APE of 0.21, a standard deviation of PE of 0.35, 3.0% of nucleotides with a PE over 1, and a highest PE of any nucleotide of 1.6. SiRNA portions of the sequences are boxed in the figure.

The sequence of the Clas-GyrAmmck-5′ insert (SEQ ID NO:47) is shown below (target sequence shown in bold):

gcatggcaatgtacggcgtaataaactttctgattttattcaaatcaat
cgtagtggtaagattgcgatgaaattagattcaagagatgagattcttt
tttaagaatcccatctcttgaaacattccctcatgcgcaataaccacca
cccatattatttgataacccacagaaggttcaaatgctgtacaccccat
gc

The sequence of the Clas-GyrAmmck-3′ insert (SEQ ID NO:48) is shown below (target sequence shown in bold):

gagatgagattctttccgttgaaacctgtacacaagaaaatgatatatt
gttgactactaaacttggacaatgtgtccgctttccgatttctgctatt
tttatagcagcaatcggaaagctaacacacttgtcccaagtggccgtag
ttccaaactatcatttcgccctgtacggtaaacacggaaagaaaacatc
tc

FIG. 19 shows CS7 (Panel B). This insert targets the callose synthase 7 gene of Nicotiana benthamiana. ΔG of CS7 is-86.4 kcal/mol and it has an APE of 0.24, a standard deviation of PE of 0.36, 5.6% of nucleotides with a PE over 1, and a highest PE of any nucleotide of 1.5. The targeted sequence of the CS7 gene is similar to a sequence of the orthologue from citrus (CscalS7), which is responsible for producing callose in sieve elements in response to pathogens. SiRNA portions of the sequences are boxed in the figure.

The sequence of the CS7 insert (SEQ ID NO:49) is shown below:

agaattatggagcggtcaagctcacacatcgtcacactttttatgtggt
gggctcagccaaaactgtatgtaggaagaggcttgcatgagggcatgtt
tttacatgccatcatgcaagccagacgactatcatacagttgaccgctg
atccaactcataaaagtcaaaatgattgtacaattgattgctctcaatt
ct

FIG. 20 shows insert Erwinia GyrA (Panel B). This insert targets the GyrA gene of Erwinia. ΔG of Erwinia GyrA is-90.8 kcal/mol and it has an APE of 0.13, a standard deviation of PE of 0.29, 4.0% of nucleotides with a PE over 1, and a highest PE of any nucleotide of 1.5. SiRNA portions of the sequences are boxed in the figure.

The sequence of the Erwinia GyrA insert (SEQ ID NO:50) is shown below (target sequence shown in bold):

ccgaccgcgcagcgccggtattattgccgtcaatctgcgtgatgacgat
gaactgatcggcgtgtcgctgacgaacggtagtgatgaagcgatgctgt
tttcagcatcacttcatcactaaaaagacgtacagtgacacataagatc
acttaatatcatccgtatacatgatgcaaaccaattggtgttgtaggtt
gg

All of the inserts mimicking structure 1 described in this section were inserted between 2219 and 2281 of CYVaV after removal of 2220-2280 of CYVaV. The inserts were stable in N. benthamiana after 16 weeks (the entire duration of the experiment) except that Erwinia GyrA was stable for 8 weeks (which was the entire duration of the experiment).

### G. Example of a Stable Insert Targeted Against CTV

CTV is composed of two capsid proteins and with a genome of more than 19 kb. 76 CTV isolates have been characterized, which all contain regions of conserved nucleotides. Two sequence portions (18 and 6) of a CTV isolate are identified in Table 3 below, showing fully conserved polynucleotides (underlined below) as well as less-conserved nucleotides (in bold) with other nucleotides present in some isolates (listed as identified and bolded nucleotides in each sequence from left to right). For example, in the sequence portion for CTV18 shown in Table 3, the 3 non-conserved nucleotides include, from left to right: guanine (G) which position instead includes adenine (A) in 10 CTV isolates; cytosine (C) which position instead includes uracil (U) in about half of the CTV isolates; and G which position instead includes A in 6 CTV isolates. In the sequence portion for CTV6, the 6 non-conserved nucleotides include, from left to right: G which position instead includes A in 1 CTV isolate; G which position instead includes A in 3 CTV isolates; U which position instead includes C in 3 CTV isolates; A which position instead includes G in 9 CTV isolates; U which position instead includes C in 1 CTV isolate; and A which position instead includes G in 1 CTV isolate.

TABLE 1
Sequence Portions of CTV Isolates

| | CTV | Sequence (conserved nucleotides | Non-conserved nucleotides |
|---|---|---|---|
| # | Position | in known CTV isolates underlined): | (bolded in sequence): |
| 18 | 15173 | UCCGUGGACGUCAUGUGUAAG | G: A in 10 isolates |
| | | (SEQ ID NO: 89) | C: U in ~half isolates |
| | | | G: A in 6 isolates |
| 6 | 17856 | GGAAGUGAUGGACGAAAUUAAUGA | G: A in 1 isolate |
| | | (SEQ ID NO: 90) | G: A in 3 isolates |
| | | | U: C in 3 isolates |
| | | | A: G in 9 isolates |
| | | | U: C in 1 isolate |
| | | | A: G in 1 isolate |

FIG. 11A shows the sequence and secondary structure of a hairpin-like structure called mmck6.2. This insert includes a sequence targeting CTV, which is the 27 bases extending on the right hand side of the insert from the bottom of the insert to just below the apical loop as the insert is shown in FIG. 11A. The MFE (alternatively called ΔG) of the insert is-29.64 kcal/mol as determined by the RNAfold software. The APE is 0.23.

The sequence of mmck6.2 is shown below (SEQ ID NO:91). The targeting sequence (intended to produce siRNA) is underlined.

gguuucaguauauuucgucaucaccuuccuaacggaagugauggacgaa
auuaaugaaacc

To investigate the stability of mmck6.2 in CY1, mmck6.2 was inserted into the CY1 genome by removing the original viral sequence between positions 2220 to 2280 and inserted mmck6.2 between positions 2219 and 2281. The resulting recombinant CY1 was then inoculated into Mexican lime citrus plants by vacuum-infiltration. Three months post-inoculation, RT-PCR analysis was performed of the systemic leaves of the inoculated plants to identify recombinant CY1 positive plants. The viral fitness and the stability of the insert in the positive plants was monitoredover a long period of time (30 months at the time of writing).

After one year post-inoculation, RT-PCR analysis and Sanger sequencing were performed of the systemic leaves of the positive plants. The results (FIG. 11B) showed that all 4 positive plants have a single RT-PCR product. The Sanger sequencing of the RT-PCR product confirmed that the insert is intact. This suggests that mmck 6.2 is stable in the CYVaV1 genome and can be maintained over an extended period of time in citrus plants. The polynucleotide sequences shown in FIG. 11B are SEQ ID NO:92 (Analysis of #7) and SEQ ID NO:93 (Analysis of #20). The sequence of mmck6.2 (SEQ ID NO: 91) is underlined:

SEQ ID NO: 69:
ttcgttgguuucaguauauuucgucaucaccuuccuaacggaagugaug
gacgaaauuaaugaaaccagttaatg
SEQ ID NO: 70:
agcttcgttgguuucaguauauuucgucaucaccuuccuaacggaagug
auggacgaaauuaaugaaaccagttaatgta

FIG. 11C shows: Panel A: Northern blot of RNA extracted from two infected citrus plants after ~one year of infection. Panel B: Reverse-transcriptase (RT)-PCR was used to amplify a segment of the infecting VIGS vector. For this plant, 10 leaves were assayed. Only a single product of the correct size for the vector with the insert was found. Panel C: Total RNA from young leaves of the infected plant were subjected to batch sequencing of the insert region. Data is from 15-month citrus showing no indication of single “double peaks” that would denote base changes or multiple double peaks that would indicate a portion of the population contain deletions. In addition, at least 10 clones were generated for individual sequencing and no changes from the initial VIGS vector must be found. SEQ ID NO: 35 shown in Panel E is missing the first G of the insert only because of the boundaries of the screenshot. The first peak in the insert band (not included in the sequence) is this missing G. Panel D: A photograph of a mexican lime plant infected with recombinant CY1 including mmck6.2 showing a lack of symptoms of infection in the plant.

Sequencing was repeated at 21 months and at 30 months post infection. At 21 months post infection, three of four mother trees still containing progeny of the CY1 vector with the Mmck6.2 insert. In one of the three mother trees, the insert had a single point mutation. At 30 months post-inoculation, samples were collected from different locations from four mother trees. Loss of insert was observed in samples taken near the bottom of two mother trees but inserts were recovered with varying degrees of mutation from samples taken near the top and bottom of the trees. Two other mother trees retained the insert all locations, with the inserts recovered from one tree showing a few base mutations and two positive samples from the other tree exhibiting minimal base mutation.

Real time quantification by stem-loop RT-PCR (using purified LMW RNA) was conducted to confirm the presence of the intended siRNA product in two N. benthamiana plants infect by CY1 vectors carrying a Mmck6.1 insert and a Mmck6.2 insert and in four citrus plants infected with a CY1 vector carrying the Mmck6.2 insert. The citrus plants had been infected for about 15 months at the time of the assay. Sequence fragments detected by this assay must end with the correct last six bases at the 3′ end, which is TAATGA. Given that RNA viruses mainly generate 21-24 nucleotide siRNA's, it is expected that sequences detected by this assay are the intended siRNA. Other effective siRNA may also be present but are not detected by the assay. For the results provided in FIG. 29, leaves were collected randomly from various oarts of the plants and combined for siRNA analysis. A mock vector and wild type CY1 were used as negative controls. As shown in FIG. 29, the intended siRNA were detected in both citrus and N. bentamiana, with the amount detected being higher in N. bentamiana. For the results provided in FIG. 30, young and mature citrus leaves were collected and analyzed separately. There was no consistent correlation between leaf stage and siRNA levels. Wild type CY1 and PsyE (a CY1 vector carrying an unrelated siRNA insert) were used as negative controls . . .

### H. APE of Exemplary Inserts

FIG. 12 Panel A show a flow chart for how the data in Panel B was generated. The predicted structure is determined by entering the sequence for the insert into RNAfold. The RNAfold software produces a list of positional entropy values of the individual bases. The positonal entropy values are given a grey scale and sorted from highest PE (darkest grey) to lowest PE (light grey)

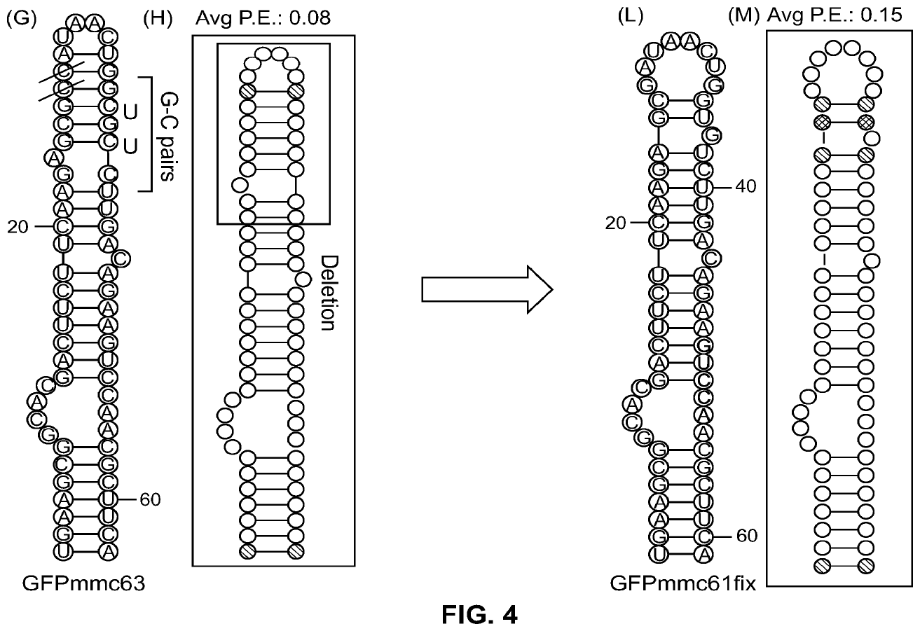

APE values for various hairpin-like structures (and some additional non-hairpin-like structures) inserted into CY1 are shown in Panel B. Sorted positional entropy is shown for each insert (dark grey to lightest grey). The first four hairpin-like structures (boxed) were duplicates of naturally occurring hairpin-like structures 1 through 4 of CY1 (see FIG. 39). Other hairpin-like structures and non-hairpin-like inserts are arranged in order of descending APE (from 1.30 to 0.07). Mimic hairpin-like structures begin with a number (1 or 4) that identifies the hairpin-like structure of CY1 being mimicked. Thick lines beneath each lane denote the length of each insert (number of nucleotides shown at right). Single and double asterisks denote inserts that were not stable in the VIGS vector. The double asterisk denotes an unstable hairpin-like structure that had three consecutive G:C pairs at the base and five consecutive G:C pairs in the apical region contributing to the very low APE of 0.07. Eliminating two nucleotides on the 5′ side and changing 2 nucleotides from C to U on the 3′ side in the apical region generated a stable hairpin-like structure (FIG. 4) without 5 consecutive apical G:C pairs and the APE value changed to 0.15 from 0.08. The thick black arrow denotes unstable hairpin-like structure (M2250gfp30ext) with a relatively low APE value of 0.39 that contained a clustering of higher entropy nucleotides.

### I. Example of a Stable Hairpin-Like Structure (GFPmmck59)

FIG. 23 shows the sequence and secondary structure of GFPmmck59. This insert has 59 nucleotides including a region targeted against green fluorescent protein (GFP) expression. Some plants or recombinant pathogens used in experimental trials have been modified to express GFP. Suppressing GFP expression can indicate replication, movement or effectiveness of a vector with a GFP suppressing insert.

This insert is stable. The minimum free energy (MFE) is-30.20 kcal/mol. GFPmmck59 has 0 (0%) bases with positional entropy (PE) greater than 1.0. The APE is 0.07 and the highest PE of any base is 0.995. The standard deviation of PE is 0.21.

The sequence of GFPmmck59 is shown below (SEQ ID NO:53). The first 27 bases of the sequence are the targeting sequence.

tgaagcggcacgacttcttcaagagcgataactcgccttgacagaagtc
caacgcttca

J. Example of an Unstable Hairpin-Like Structure with Low APE (GFPmmck63)

FIG. 24 shows the sequence and secondary structure of GFPmmck63. This insert has 63 nucleotides including a region targeted against green fluorescent protein (GFP) expression. Despite having an APE of 0.08, no bases with PE greater than 1, and a standard deviation of PE of 0.22, this insert is relatively unstable. The MFE is-36.60 kcal/mol.

One region of this insert has 5 consecutive G-C pairs. Deletion of two nucleotides on the 3′ side and changing two nucleotides from C to U on the 3′ side in the apical region eliminated the 5 consecutive G-C pairs (FIG. 4). The resulting insert, GFPmmck61fix, had fewer G-C pairs and was stable. GFPmmck61fix has an APE of 0.15, a maximum PE of 1.01, 61 nucleotides, one base (1.6%) with PE greater than 1, a standard deviation of PE of 0.28 and an MFE of −26.4 kcal/mol. This example suggests that a large number (e.g. more than 4) consecutive G-C pairs leads to instability, even in a hairpin-like insert meeting other guidelines.

The sequence of GFPmmck63 is shown below (SEQ ID NO:54). The first 30 bases of the sequence are the targeting sequence.

Tgaagcggcacgacttcttcaagagcgccataactggcgccttgacaga
agtccaacgcttca

### K. Second Example of an Unstable Hairpin-Like Structure (M2250GFP30ext)

FIG. 22 shows the sequence and secondary structure of M2250GFP30ext. This insert is relatively unstable. The MFE is-27.3 kcal/mol. M2250GFP30ext has 9 (13.8%) bases with positional entropy (PE) greater than 1.0. The APE is 0.39 and the highest PE of any base is 1.97. The standard deviation of PE is 0.52.

The sequence of M2250GFP30ext is shown below (SEQ ID NO:52):

ggttagggtaactcactgaagcggcacgacttcttcaagagcgccattc
agtgagcaacctaacc

L. Third Example of an Unstable Hairpin-Like Structure with Low APE

FIG. 27 shows the sequence and secondary structure of BBLv2-1. BBLv2-1 has 198 nucleotides. 3 nucleotides (1.5%) have PE greater than 1. The APE is 0.18 and the standard deviation of PE is 0.3 but this insert is unstable.

The sequence of BBLv2-1 is shown below (SEQ ID NO:57):

cuggaaaauucgauuacucaaagugccuaaaaucguaacaacuuguaug
aucuauaggccuggauccaaacaauacguggcucaaauacuugagaaau
uuuuuucucauguauuugagccuaaaccuugauuuggauccaucuccua
ucgaacaccaaguguuaaacuuuuuagcagagggaguaaucgaaauucc
ag

The sequence of BBLv2-1 after deletion is shown below (SEQ ID NO:58):

cuggaaaauucgauuacucaaagugucgaaauuccag

An analysis of BBLv2-1 indicated that modification of the vector in an infected plant involved removal of a region having a bulge (no bases on one side, 1 base on the other side), 5 A-T base pairs (A-U base pairs are included as A-T base pairs), an asymmetric loop with 7 bases (counting both sides) and 3 more A-T base pairs. The AG of the asymmetric loop was positive (2.7 kcal/mol) and the ΔG of the region from the bulge to the asymmetric loop (including both the bulge and the asymmetric loop) had a positive ΔG of 0.4 kcal/mol. Without intending to be limited by theory, instability of this inert may have been caused by the locally positive ΔG of this region.

This example suggests a number of potential design parameters such as that the number of consecutive A-U base pairs should be less than 5 or that the ΔG of a region including two loops and the stack between them should not be positive.

### M. Example of a Hairpin-like Structure of CTV.

The stability of hairpin-like structures that mimic hairpin-like structures in the CY1 and OULV genomes has been previously demonstrated by their ability to remain stable, for example when inserted to replace the sequence between 2220 to 2280 (i.e. inserted at 2219/2281) or inserted at 2304/2305, 2319/2320 or 2330/2331 of CY1. To investigate whether this rule also applies to hairpins that originate from an unrelated virus, every 2 kb of the CTV genome was subjected to secondary RNA structure prediction using mfold software obtained from the “UNAFold Web Server” (http://www.unafold.org/mfold/applications/rna-folding-form-v2.php) in search of hairpins that resemble a naturally-occurring hairpin of CYVaV1 in terms of size and shape. As a result, a 128 nt hairpin-like structure, CTV-insert-natural-V2.2, was identified which forms a stack-loop structure with internal asymmetrical loops. The overall structure of this hairpin resembles a smaller version of a hairpin-like structure 1 of CY1. CTV-insert-natural-V2.2 has a ΔG of −43.6 kcal/mol (mfold) or −46.96 (RNAfold) and APE of 0.359.

The sequence of CTV-insert-natural-V2.2 is (SEQ ID NO:55):

ggggguuuauguuuggcaaagaaaguguuggaacuguuagucaagcggg
ugguugaaucguuuucucguuugaagcggaaaaccgcucguuuaacguc
cuucgcuaauuuguugcuugcgaggcucuc

FIG. 25 shows the sequence and secondary structure of CTV-insert-natural-V2.2 as predicted by “mfold” and drawn by “RNA2Drawer.”

To test the stability of CTV-insert-natural-V2.2 in CY1, this hairpin-like structure was inserted into the CY1 genome by replacing the original viral sequence between 2220 to 2280 of CY1 with CTV-insert-natural-V2.2. The resulting recombinant CY1 was then inoculated into N. benthamiana plants by syringe infiltration and viral fitness as well as the stability of the insert was monitored over a period of time. Three weeks post-inoculation, RT-PCR analysis and Sanger sequencing were performed of the systemic leaves of all symptomatic plants. The results (FIG. 26) show that all 9 symptomatic plants tested have a single RT-PCR product that migrates slower than CYVaV1 wt control, suggesting the presence of recombinant CY1 with CTV-insert-natural-V2.2. The Sanger sequencing of the RT-PCR product confirmed that the insert is intact in all 9 symptomatic plants.

### N. Example of a Hairpin-like Structure Encoding a Peptide.

FIG. 28 shows modifications at the end of a hairpin-like region in the CY1 p81 (RdRp) ORF to remove the region from 2159-2197 (including the p81 stop codon at 2160-2162) and insert a T2A self-cleavage peptide (including a T2A cleavage site) followed by a peptide to be expressed in the host plant, a new stop codon, and additional nucleotides to provide a stable hairpin-like insert.

The sequence of the wild type CY1 2136-2218 region shown in the Figures, which is a hairpin-like structure including the C—terminal of the RdRp (p81) ORF, is presented below (SEQ ID NO: 94):

ACGAGGACUUAGGCGUCCUAGGAUGAAUAGGGUCAUUGGUUUACCGAUG
AUACCUGUUCAGAAUAGGAUUGCUCGAGCUU
CGU

The underlined UGA in SEQ ID NO: 72 is the native CY1 stop codon.

The sequence of the modified CY1 shown as P81T2A in the FIG. 28, starting at position 2136 of CY1, is presented below (SEQ ID NO: 95):

ACGAGGACUUAGGCGUCCUAGGAggaagcggagagggcagaggaagucu
gcuaacaugcggugacgucgaggagaauccugaccuugacgauccgaug
uucuaaccgaagucaccgaaggccagcggacccccucugaccuaccuga
uuccugAUAGGAUUGCUCGAGCUUCGU

The underlined UGA in SEQ ID NO: 95 is the P81T2A stop codon.

The sequence of the T2A self-cleavage peptide shown in the Figures, is presented below (SEQ ID NO: 74):

G S G E G R G S L L T C G D V E E N P G P

The “/” in SEQ ID NO: 74 indicates the T2A cleavage site.

The sequence of the modified CY1 shown as P81T2A_PEP6His-tag in the figures is presented below (SEQ ID NO: 75):

ACGAGGACUUAGGCGUCCUAGGAggaagcggagagggcagaggaagucu
gcuaacaugcggugacgucgaggagaauccuggaccuuuuaggcuaaag
uuucaucaccaccaccaucaccacugaccggugauaguguuggugauga
aacuuuaggauaaacgauccgauguucuaaccgaagucaccgaaggcca
gcggacccccucugaccuaccugauuccugAUAGGAUUGCUCGA
GCUUCGU

The underlined uuuaggcuaaaguuucau in SEQ ID NO: 75 encodes the PEP6 peptide. The underlined uga in SEQ ID NO: 75 is a stop codon. The intervening caccaccaccaucaccac in SEQ ID NO: 75 encodes the (6×) His-tag.

The sequence of the T2A self-cleavage peptide and the cleaved protein shown in the Figures, is presented below (SEQ ID NO: 76):

G S G E G R G S L L T C G D V E E N P G/P F R L K
F H H H H H H H

The “/” in SEQ ID NO: 76 indicates the T2A cleavage site. The underlined F R L K F H in SEQ ID NO: 76 is the PEP6 peptide. The downstream H H H H H H in SEQ ID NO: 76 is the (6×) His-tag. The complete cleaved peptide is a P-PEP6His-tag.

In the sequences above, uppercase letters indicate parts of the native CYVaV sequence. Lowercase letters indicate parts of the heterologous (inserted) sequence.

N. benthamiana plants were infected with a CY1 vector with the P81T2A_PEP6His-tag insert by way of agroinfection. The vector was stable (replicated in the host plant without losing the heterologous insert) for the duration of a 6 week trial. The T2A functionality was confirmed in-vivo using CY1 with P81-His and CY1 with P81T2APEP6His-tag. In Western blots, His-tagged p81 was detected but p81T2APEP6His-tag was not detected. This indicates that the T2A-fusion protein functioned properly in the plants. The P-PEP6histag peptide was selected to facilitate experimental assays but may be replaced with another antimicrobial peptide.

The P81T2A_PEP6His-tag insert was designed according to “mimic” principles. The first 201 nucleotides of the inserted sequence form a hairpin-like structure, with the final 2 nucelotides (UG) corresponding in position to the bases at 2157 and 2158 of wild type CY1, which were left unpaired with the deletion of the 2159-2197 region. The stop codon of the insert is placed in the apical loop. The structure formed by the first 201 nucleotides has an MFE of −98.6 kcal/mol as calculated by the RNAfold web server. The average positional entropy (APE) is 0.115.

The entire hairpin-like structure that replaces the structure formerly at positions 2136-2218 of CY1 has a length of 247 nt, an APE of 0.13, a maximum P.E. of 1.1, one base (0.4%) with a PE>1, a standard deviation of PE of 0.21, 17 consecutive base pairs in one region, and an MFE of −123.3 kcal/mol, which deviates from the formula presented herein by −12.7 kcal/mol. Another long insert, CY2_2163T2APEP6His, had 284 nucleotides, an APE of 0.22, a maximum P.E. of 1.8, 21 bases (7.4%) with a PE>1, a standard deviation of PE of 0.39, and an MFE of −144.5 kcal/mol, which deviates from the formula presented herein by −17.7 kcal/mol. Both of these inserts are stable. In contrast, an unstable T2A peptide insert had 120 nucleotides and an MFE which devitated from the formula by −31.5 kcal/mol. Collectively these examples suggest that long inserts, e.g. with 150 nucleotides or more, may have an MFE that deviates from the formula by about 20 kcal/mol, particularly by being more negative.

### O. Targeting the Plant Susceptibility Gene ML06 for Disease Resistance.

Plant pathogens pose a significant threat to crop yield and productivity by exploiting the susceptibility(S) genes inherent in plants to facilitate their infection. Well-studied S genes include botrytis-induced kinasel (BIK1), Sugars Will Eventually be Exported Transporters (SWEETs), Lateral Organ Boundaries 1 (LOB1), Mildew Locus O (MLOs) such as MLO6 and MLO7, Downy mildew Resistance (DMRs) such as DMR6, Defense No Death (DNDs) genes such as DND1, Translation Initiation Factors (eIF4E), DspA/E-interacting proteins from Malus (DIPM1-4), and Enhanced Disease Resistance 1 (EDR1). Perturbation of these S genes through mutagenesis, genetic engineering, or exploitation of natural variants confers resistance to a wide range of plant diseases. However, complete knockout of these genes can result in unexpected stunt phenotypes, leading to significant yield and quality loss. To address this challenge, a VIGS vector was used to partially silence host susceptibility genes. In the examples described herein, a CY1 based vector is engineered to target an MLO or DMR gene.

In the examples described herein a heterolougs RNA segment is added to the CY1 backbone. In particular, a wildtype hairpin-like region from 2220-2296 of the CY1 backbone as presented herein is removed and replaced with the heterologous RNA segment. In other examples, a different insertion site may be used, with or without deletion of a wildtype hairpin-like region from the backbone. In other examples, a different viral backbone may be used as appropriate for a host plant. For example a CTV vector may be used to treat citrus trees or a TRV vector may be used to treat various plants that TRV is known to infect. In other examples, other susceptibility genes may be targeted with a ULV, Class 2 ULV or CY1 vector.

One of the most pervasive plant diseases, impacting a wide range of plant species (i.g. over 650 species of powdery mildew fungi that affect about 10,000 plant species). Grapevine Powdery Mildew is a predominant fungal disease affecting grapevines globally and can lead to yield losses of up to 75-100%, particularly during high disease-pressure years. Numerous grapevine varieties lack genetic resistance, necessitating extensive fungicide programs in viticulture, with an annual average of 19.5 kg/ha of active ingredients. The collective expense of fungicide application ranges between 9% and 20% of the total cultural cost. This costly and necessary practice heightens the risk of resistance development in E. necator populations.

A universal weapon to defeat Powdery Mildew disease is to silence Mildew Resistance Locus O genes (MLO). MLO serves a vital role as a negative regulator, influencing defense activation by impedgin the transport of defense compounds to nascent papillae. This interference assists germinated Powdery Mildew pathogen spores in preventing the fomratio of a Haustorium for nutrient uptake.

Knocking down or knocking out one or more specific MLO genes represents a strategy for protecting plants against Powdery Mildew infections.

Stable inserts were designed to target the MLO6 gene in N. Benthamiana. The N. benthamiana MLO6 gene was selected to validate this approach. This approach involved harnessing the CY1-induced host MLO gene to mitigate Powdery Mildew Disease in the model plants.

Three CY1 constructs containing the inserts target at MLO6 expression were engineered and used to infect host plants, which were then evaluated for their disease resistance again the tobacco powdery mildew isolate Golovinomyces cichoracearum (Gc) SICAU1. Visual inspection of leaves for symptoms (white fungal mass) after 8 weeks indicated that plants infected with the CY1 constructs containing inserts targeted against NbMlo6 had enhanced resistance to powdery mildew realtiove to plants ionfected with a control (wildtype CY1).

The three CY1-based RNAi vectors were designed by using a modified ~97 nucleotide RNA sequence from the N. benthamiana MLO6 gene, which were engineered based on the hairpin mimic structure described herein (FIG. 32). The CY1 wt ΔG was-77 kcal/mol. The ΔG of CY1.NmMloJH was −71.10 kcal/mol. The ΔG of CY1.NmMlo1 was −72.90 kcal/mol. The ΔG of CY1.NmMlo2 was −70.50 kcal/mol.

NbMloJH has an average APE of 0.25; maximum PE of 1.6; length of 198 nucleotides; 13 bases (6.6%) with PE greater than one, a standard deviation of PE of 0.39 and an MFE of −80.7 kcal/mol. The sequence of NbMloJH is SEQ ID NO: 77:

ACCTGGGGATGACCTCTTTTGGTTTAATCGTCCTCGTCTCATTCTTTAT
CTCATTAATTTTGTCCTCTTTCAGAATGCTTTTCAATTGGCCTTCTTTT
TTTAAAGAAGCCCAATTGAAAACTTAAACTGTAAAGAGGACTCTTATTA
AGGAAATTAGAATAGACCACCGACGA TAA GAGTAAAGAGGTCATGCC
AGGT

NbMlo1 has an average APE of 0.23; maximum PE of 1.6; length of 198 nucleotides; 13 bases (6.6%) with PE greater than one, a standard deviation of PE of 0.38 and an MFE of −82.8 kcal/mol. The sequence of NbMlo1 is SEQ ID NO: 78:

ATGACCTCTTTTGGTTTAATCGTCCTCGTCTCCTTCTTTATCTCATTAA
TTTTGTCCTCTTTCAGAATGCTTTTCAATTGGCCTTCTTTGCTTGGACT
TTTGTCCAAGAAAAGAAGGCCACCACACAAATGCATTCTGACACAAGGA
CCAACTTCTGAGAAAAGCTCAGAGACAGGCAACTTAAACCAAAACGGTC
AT

NbMlo2 has an average APE of 0.17; maximum PE of 0.97; length of 198 nucleotides; 0 bases (0%) with PE greater than one, a standard deviation of PE of 0.26 and an MFE of −79.8 kcal/mol. The sequence of NbMlo2 is SEQ ID NO: 79:

GTTGATTACTTAACCCTAAGGCATGGATTTATCATCGCGCATTTGGCAC
CTCAGAGCCACGTAAAGTTTGATTTCCAAAAGTATATCAACAGGTCACT
TTTGTGACCTATTGATATACTTCAGACTAATACAAACTTTAAACCGGCT
CAGACGTACAAATCGCGCCAAATAAACCAACAATTAGGGTTAAGCATCA
AC

The DMR6 gene is also an effective suspceptibility gene. Targeting DMR6 and sequences of DMR6 in various plants are described, for example, in US 2021/0071195, Soybean Plants Resistant to Phytophthora Sojae, which is incorporated herein by reference.

In experiments, three inserts were designed targeting the N. benthamiana DMR6 gene (NbDMR6). The three inserts, DMR6.1, DMR6.2 and DMR6.3, are shown in FIG. 31. Other inserts target at DMR6 genes in citrus, grape and cannabis are also shown in the FIG. 31.

Eighteen N. bentamian plants for each CY1 construct were agroinfiltrated with CY1 containing inserts to produce NbDMR6 silencing siRNA, employing hairpin-like structures as described herein. Three weeks after agroinfiltration, the expression levels of the NbDMR6 gene were quantified using real-time PCR (qPCR). The data analysis reveladed that NbDMR6 gene expression was reduced by about 55% in plants infected by CY1-DMR6 compared to the control plants.

Three systemic leaves on each plant, previously infected with CY1 carrying NbDMR6 silencing inserts, were subsequently challenged with 0.0001 (OD600) of P. syringae pv tabaci through infiltration in a 10 mM MgCL2 solution (three plants for each CY1 siRNA construct). As a control, plants infected with wild-type CY1 were also subjected to bacterial infiltration. Four days after the challenge, the disease caused by P. syringae was assessed by visual inspection. Leaves in plants treated by CY1-DMR6 shows minimal symptoms whereas leaves in plants treated with a wildtype CY1 control showed severe necrosis (large areas of white discoloration). The bacterial titer in the infiltrated areas was determined by plating. The results demonstrated that the bacterial levels in plants treated with the CY1-DMR vector were reduced by nearly 10.9 fold relative to the control plants.

The sequence of DMR6.1 (SEQ ID NO: 80) is:

GGCCTCAATTTACCAGTAGAAGAGAAACTAAAATTATATTCAGATGATC
CTTCAAAGACCATGAGATTATCAACAAGTTTTAATGTTAGGAAGGAGAC
ATTTTTGTCTCCGTCCTAACATTATGTTAATGTATGATAATCTGGCGGG
TCTGTGTAGTTCATCGAATCCCCATTTTGTTAAAATTCTACTGGTACTT
GAGGCC

The sequence of DMR6.2 (SEQ ID NO: 81) is:

CTCTAGAGAAGTATGCTCCTGAATGGCCTTCTAATCCATCATCTTTCAG
GGAAATCGTGAGCAGATATTGCAGGGAAATTCGTCAACTCGGATTTAGT
TTTCTAAATCGGAGTTGACGAACCAAAACTGCCAATATCTGAAAAACGA
TATCACTTAAGATATGGTAAGAGAAGCCAAAATGGAGCATATTTACTAG
AG

The sequence of DMR6.3 (SEQ ID NO: 82) is:

GCCATAGCAGAAAGCCTGGGGTTAGATAAAGAGTGTATAAAAGATGTAT
TGGGTGAACAAGGACAACATATGGCTATCAATTATTATCCTCCTTGTCT
TTTGACAAGGTGGATAATAATTCTCCCTCCATTATGTTGTCAAAAGTTC
AGCCGATGATCATTATAATTATCTTTTCTCCAACCAGGCTTTCTATATG
GC

The sequence of DMR6.CITRUS (SEQ ID NO: 83) is:

GACCCAAATGCCCTTACCATTTTATTGCAAGATTTAGAAGTGGCAGGTC
TTCAAGTTCTCAAAGACGACAAATGGGTTGCTGTTAATCCCCTTCCTAT
TTTTAGGAAGCGGATTAACAGCTTTGAAATTATGTTGTCTTCCTTGAAT
TCGATGAATGTCATTCTTTCTTCTTGAAATCTCCTGGTAAGGGTAATGG
GTC

The sequence of DMR6.GRAPE (SEQ ID NO: 84) is:

CCTCAACATGGTTGAAGGGAGTAAAAGATTTTTCGAGCTCCCTTTCGAG
GAGAGATCCAAGTACATGTCGACTGATATGTATGCCCCAGTTAGGTATT
TTTATATCTATCTGGGGTATACCCACAAAGTACGATATGTAAAACGGAT
CACTACTAAAAGGAGCTAATTAAAATATTTGGGACCCTTCAACCACTTG
AGG

The sequence of DMR6.CANNABIS (SEQ ID NO: 85) is:

CTTCCACATGAGGAGAGAGCTAAGTACATGTCCTCTGATATGAAAGCTA
AAGTGCGATATGGAACAAGCTTTAACCAAACTAAAGACAAAGTCTTTTT
TTTAAAAGACATTGTCTTTAGTAACCCATAAAGCTTGTTCTTAAATCGC
TCTATAATTTCAATCACCTCGACATTACAATCCTCTCTCCTCAATGGAA
G

P. Insert Stability for Animal Vectors-Poliovirus and Coxsackievirus B3 (CVB3) live attenuated vaccines.

Live attenuated vaccines induce the most complete and durable immune response, including local and systemic responses, as they closely recapitulate the life cycle of the pathogenic viruses. In addition, they are relatively easy to propagate and administer. Currently, live attenuated vaccines against poliovirus (oral poliovirus vaccine, OPV) are applied on a global scale, and they are envisioned as the cornerstone of the Global Polio Eradication Initiative.

Unfortunately, the currently available anti-poliovirus vaccines, including the newly developed OPV strains (nOPV), have suboptimal properties which in combination with the administrative difficulties of organizing effective vaccination campaigns has led to repeated missed deadlines for poliovirus eradication. The original OPV developed by Albert Sabin is a combination of three attenuated poliovirus strains. In addition to inducing gut mucosal immunity required for the interruption of viral transmission, it is inexpensive, can be administered by minimally trained personnel, and the replicating vaccine may spread beyond the primary vaccine recipient increasing the vaccination coverage, which makes this vaccine the vaccine of choice in limited resource settings. Yet, in about 1 per million of the primary vaccine recipients it induces vaccine-associated paralytic poliomyelitis (VAPP), and OPV-derived viruses can establish circulation in under-immunized populations, generating so-called circulating vaccine-derived polioviruses (cVDPV) which may also cause the disease. In fact, currently most of the registered cases of paralytic poliomyelitis are attributed to cVDPV rather than to the remaining circulation of the wild type viruses.

Thus, the development of improved live polio vaccine strains is considered a high priority. The current approaches include a combination of the stabilization of a secondary structure element in the non-translated genome region, rearrangement of an important cis-acting RNA signal, and the introduction of mutations improving the fidelity of the viral RNA polymerase. These features decrease the possibility of reversion to the pathogenic phenotype and the recombination with other enteroviruses. Recently, a newly developed vaccine strain of poliovirus type 2 with several features for increased stability was introduced into immunization practice (nOPV2). Yet, in spite of the promising clinical trial results, the recent data register both the emergence of nOPV2-derived cVDPV and the associated cases of paralytic disease. Thus, the strict requirements for stable and safe live poliovirus vaccine strains have not been met.

Data herein demonstrate that unprecedented stability of inserts in the 5′ non-translated region of enteroviruses, including poliovirus and CVB3. Poliovirus and Coxsackie B 3 virus (also an important human pathogen) were generated with inserts harboring target sequences for miRNAs specifically expressed in neuronal and cardio tissues. Introducing miRNA target sequences in the viral genome allows cell-type-specific inhibition of replication. This is a promising direction for the design of safe live attenuated vaccine strains provided that methods, for example as described herein, for stabilizing the miRNA targeted inserts are applied.

The stabilized inserts were 99-100% stable over at least 10 passages of these viruses in cell culture, i.e. over multiple replication cycles. Control inserts designed without considering the insert stability parameters were lost within the first passages, in accordance with previous reports.

The approach of stabilizing inserts in the genomes of RNA viruses was tested on two enteroviruses, poliovirus and Coxsackievirus B3 (CVB3) (family Picornaviridae, genus Enterovirus, species C and B, respectively). Enteroviruses are positive-strand RNA viruses with the genomes of ~7500nt. The enterovirus RNA codes for one polyprotein and contains an extensive 5′ non-translated region (5′UTR). There is a poorly conserved stretch of ~100 nucleotides between the end of IRES and the beginning of the viral ORF (FIG. 33), which is believed to be scanned by the ribosomal 40S subunit during translation initiation.

Hairpin-like inserts containing target sequences were designed for the neuronal and cardio tissue-specific miRNAs (hsa-miR-9 and hsa-miR-150) for insertion into this region in poliovirus and CVB3 genomes, respectively. The insertion of miRNA target sequences is a promising approach for vaccine strain attenuation but is difficult to implement in positive RNA viruses because they readily removed foreign inserts in the genome in previous studies. For both viruses, two insertions were designed-one according to the methods described herein which includes adjustment of the hairpin stability and in these examples surrounding sequence modification, and the control constructs with straightforward hairpin insertion in the viral genome (FIG. 33).

The corresponding RNAs were synthesized using a T7 transcription system and transfected into HeLa cells. All viruses were viable and showed plaque phenotypes similar to that of the wildtype viruses, except for the CVB3 control construct which produced noticeably smaller plaques.

The viruses were passaged 10 times in Hela cells (each passage comprised multiple cycles of replication). The total viral RNAs from passages 0 (the recovered virus after RNA transfection), 5, and 10 were extracted, and the region encompassing the inserted sequences was amplified by RT-PCR and sequenced.

### Poliovirus

The secondary structure of the insert (has-miR-9-5p MIMAT0000441), the backbone polio virus and the modified polio virus (after replacement of wildtype bases 620-679 with the insert has-miR-9-5p MIMAT0000441) is shown in FIG. 38. Insert has-miR-9-5p MIMAT0000441 has an APE of 0.08 (0.06 without the AU pair end), a length of 52 bases, and an MFE of −24.80 kcal/mol. For the control poliovirus construct, sequence heterogeneity was observed already in passage 0, showing extreme instability of the hairpin insert (FIG. 34). In passages 5 and 10 of the control construct only wt sequence was recovered (not shown).

On the contrary, for the designed insert in the poliovirus genome, only one nucleotide change was observed after 10 passages (FIG. 35).

The region of the vector including the stabilized heterologous insert (in bold) shown in the figures is presented below as SEQ ID NO: 86:

ACAATCACAGATTGTTATCATAAAGCGAATTCTTTGGTTATCTAGCTGT
ATGAatcacaTCATCCAGCCCGATAACCAAAGATTGAGTGTGTTTACTC
TAAGTACAATTTCAACAGTTATTTCAATCAGACAATTGTATCATAATGG
GTGCTCAGGTTTCATCACAGAAAGTGGGCGCACATGAAAACTCAAATAG
AGCGTATGGTGGTTCTACCATTAATTAC

In some figures, the sequencing test data differs from SEQ ID NO 87 by way of mutations or deletion appatent from the figures.

### CVB3

The secondary structure of the insert (hsa-miR-150-5p MIMAT0000451), the backbone polio virus and the modified polio virus (after replacement of wildtype bases 648-676 with the insert hsa-miR-150-5p MIMAT0000451) is shown in FIG. 37. Insert hsa-miR-150-5p MIMAT0000451 has an APE of 0.05, a length of 49 bases, and an MFE of −19.20 kcal/mol. The control insert in CVB3 genome was more stable than that in poliovirus RNA, but at passage 5 we observed a significant deletion, and at passage 10 all the insert and part of the surrounding sequence was absent (FIG. 36 Part A). Remarkably, the designed sequence was absolutely stable in the CVB3 genome during 10 passages (FIG. 65 part B).

The region of the vector including the heterologous stabilized insert (in bold) shown in the is presented below as SEQ ID NO: 87:

ATTGGCCATCCGGTGACCTCTCCCAACCCTTGTACCAGTGtatatCACT
AGCACAAGCATTGGGAGCGTTTATACCACTTAGCTTGAAAGAGGTTAAA
ACATTACAATTCATTGTTAAGTTGAATACAGCAAAATGGGAGCTCAAGT
ATCAA

In some figures, the sequencing test data differs from SEQ ID NO 87 by way of mutations or deletion appatent from the figures.

Thus, the results demonstrate that the method of insert stabilization can be broadly applied to positive-strand RNA viruses. The method may be used, for example, for designing stable vaccine strains with insert-derived attenuation properties.

All identified publications and references mentioned herein are hereby incorporated by reference to the same extent as if each such publication was specifically and individually indicated to be incorporated by reference in its entirety. While the disclosure has been described in connection with exemplary embodiments, it will be understood that it is capable of further modifications and this application covers any variations, uses, or adaptations following, in general, the principles of the disclosure and including such departures as come within known or customary practice within the art to which the disclosure pertains and as may be applied to the features hereinbefore set forth.

1. A ribonucleic acid (RNA) molecule derived from an RNA virus whose nucleotide sequence has been modified to comprise a heterologous RNA segment, wherein the heterologous RNA segment comprises two or more base paired regions, one or more non-base-paired regions separating the base paired regions, and an apical loop at the end of one of the base-paired regions, and wherein the heterologous RNA segment has an average position entropy (APE) in the range of 0.01 and 0.75 and a minimum free energy in kcal/mol in a range of about −5 to +15, or in a range of about −10 to +10, from −0.44 multiplied by the number of bases of the heterologous RNA segment minus 1.89 or, if the heterologous RNA segment has more than 150 nucleotides, a minimum free energy in kcal/mol in a range of about −20 to +20 from −0.44 multiplied by the number of bases of the heterologous RNA segment minus 1.89.

2. The RNA molecule of claim 1 wherein the heterologous RNA segment has an APE of less than 0.75, 0.65, 0.5, 0.4, 0.39, 0.36 or 0.32.

3. The RNA molecule of claim 1 wherein the heterologous RNA segment has an APE greater than 0.01, 0.02 0.03, 0.04, 0.05, 0.06 or 0.07.

4. The RNA molecule of claim 1 wherein the base-paired regions of the heterologous RNA segment have 19 or less base pairs or 17 or less base pairs or 13 or less base pairs or 10 or less base pairs.

5. The RNA molecule of claim 1 wherein the heterologous RNA segment has no more than 4, or no more than 3, consecutive G:C base pairs.

6. The RNA molecule of claim 1 wherein the heterologous RNA segment has no more than 20 bases collectively on both sides of the non-base paired region.

7. The RNA molecule of claim 1 wherein a) each base-paired region of the heterologous RNA segment has an APE of less than 0.8 or b) the DG of a region of the heterologous RNA segment including two loops and a stack between them is not positive.

8. The RNA molecule of claim 1 wherein the heterologous RNA segment has between 40 and 300, 40 and 200, 60 and 300 or 60 and 200 bases.

9. (canceled)

10. The RNA molecule of claim 1 wherein the heterologous RNA segment has a standard deviation of PE less than 0.5 or less than 0.4.

11. The RNA molecule of claim 1 wherein the heterologous RNA segment has a) less than 15% or less than 10% of its bases have a PE of greater than 1.0 or b) the largest PE of any of its bases is not greater than 2.0 or not greater than 1.5, or c) both.

12. The RNA molecule of claim 1 wherein the heterologous RNA segment targets a susceptibility gene of a plant.

13. The RNA molecule of claim 12 wherein the susceptibility gene is a botrytis-induced kinase 1 (BIK1) gen, a Sugars Will Eventually be Exported Transporters (SWEET) gene, a Lateral Organ Boundaries 1 (LOB1) gene, a Mildew Locus O (MLOs) gene, a Downy mildew Resistance (DMR) gene, a Defense No Death (DND) gene, a Translation Initiation Factors (elF4E) gene, a DspA/E-interacting proteins from Malus (DIPM1-4) gene, or an Enhanced Disease Resistance 1 (EDR1) gene.

14. The RNA molecule of claim 1 wherein the RNA virus is an animal virus.

15. The RNA molecule of claim 14 wherein the RNA virus is a live attenuated attenutated virus of a vaccine.

16. The RNA molecule of claim 14 wherein the RNA virus is a poliovirus.

17. The RNA molecule of claim 14 wherein the heterologous RNA segment is complementary to a microRNA of the animal.

18. (canceled)

19. (canceled)

20. (canceled)

21. (canceled)

22. (canceled)

23. The RNA molecule of claim 1 where a portion of the RNA molecule derived from the RNA virus has a sequence identity of at least 50%, at least 60%, at least 70%, at least 80%, at least 90% or at least 95% with the RNA virus.

24. The RNA molecule of claim 1 wherein the heterologous RNA segment replaces a hairpin-like region of the RNA virus.

25. (canceled)

26. (canceled)

27. (canceled)

28. (canceled)

29. (canceled)

30. (canceled)

31. A ULV, ulaRNA, Class 2 ULV, CY1, CTV or TRV vector comprising a heterologous RNA segment targeted against a host plant susceptibility gene, wherein the heterologous RNA segment mimics one or more wildtype hairpin-like regions of the vector and/or wherein the heterologous RNA segment comprises two or more base paired regions, one or more non-base-paired regions separating the base paired regions, and an apical loop at the end of one of the base-paired regions, and wherein the heterologous RNA segment has an average position entropy (APE) in the range of 0.01 and 0.75 and a minimum free energy in kcal/mol in a range of about −5 to +15, or in a range of about −10 to +10, from −0.44 multiplied by the number of bases of the heterologous RNA segment minus 1.89 or, if the heterologous RNA segment has more than 150 nucleotides, a minimum free energy in kcal/mol in a range of about −20 to +20 from −0.44 multiplied by the number of bases of the heterologous RNA segment minus 1.89.

32. (canceled)

33. (canceled)

34. A method of attenuating a live RNA virus vaccine comprising a step of providing a heterologous RNA insert, wherein the heterologous RNA insert is complementary to a microRNA of a host animal and the heterologous RNA insert comprises two or more base paired regions, one or more non-base-paired regions separating the base paired regions, and an apical loop at the end of one of the base-paired regions, and wherein the heterologous RNA segment has an average position entropy (APE) in the range of 0.01 and 0.75 and a minimum free energy in kcal/mol in a range of about −5 to +15, or in a range of about −10 to +10, from −0.44 multiplied by the number of bases of the heterologous RNA segment minus 1.89 or, if the heterologous RNA segment has more than 150 nucleotides, a minimum free energy in kcal/mol in a range of about −20 to +20 from −0.44 multiplied by the number of bases of the heterologous RNA segment minus 1.89.
